(19) United States
(12) Patent Application Publication
(10) Pub. No.: US 2006/0264496 A1
(43) Pub. Date: Nov. 23, 2006

(54) Substituted indoline and indole derivatives

(75) Inventors: Nikolay Khanzhin, Frederiksberg (DK); Mario Rottlander, Greve (DK); William P. Watson, Vanlose (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S, Valby-Copenhagen (DK)

(21) Appl. No.: 10/551,738

(22) PCT Filed: Apr. 23, 2004
(86) PCT No.: PCT/DK04/00283

§ 371 Date: Feb. 7, 2006

Related U.S. Application Data
(60) Provisional application No. 60/465,387, filed on Apr. 25, 2003.

(30) Foreign Application Priority Data
Apr. 25, 2003 (DK) PA200300631

(51) Int. Cl. A61K 31/405; C07D 403/02

(52) U.S. Cl. 514/414; 514/419; 548/465; 548/495

(57) ABSTRACT

The present invention relates to aniline derivatives of the general formula I or pharmaceutically acceptable salts thereof, [Formula (I)], and their use.

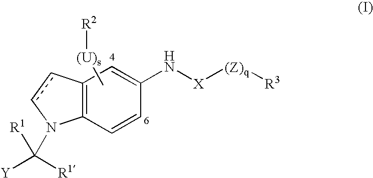

### FIELD OF THE INVENTION

The present invention relates to novel substituted indole and indoline derivatives being openers of the KCNQ family potassium ion channels. The compounds are useful for the prevention, treatment and inhibition of disorders and diseases being responsive to opening of the KCNQ family potassium ion channels, one such disease is epilepsy.

### BACKGROUND OF THE INVENTION

Ion channels are cellular proteins that regulate the flow of ions, including potassium, calcium, chloride and sodium into and out of cells. Such channels are present in all animal and human cells and affect a variety of processes including neuronal transmission, muscle contraction, and cellular secretion.

Humans have over 70 genes encoding potassium channel subtypes (Jentsch Nature Reviews Neuroscience 2000, 1, 21-30) with a great diversity with regard to both stucture and function. Neuronal potassium channels, which are found in the brain, are primarily responsible for maintaining a negative resting membrane potential, as well as controlling membrane repolarisation following an action potential.

One subset of potassium channel genes is the KCNQ family. Mutations in four out of five KCNQ genes have been shown to underlie diseases including cardiac arrhythmias, deafness and epilepsy (Jentsch Nature Reviews Neuroscience 2000, 1, 21-30).

The KCNQ4 gene is thought to encode the molecular correlate of a potassium channel found in outer hair cells of the cochlea and in Type I hair cells of the vestibular apparatus, in which, mutations can lead to a form of inherited deafness.

KCNQ1 (KvLQT1) is co-assembled with the product of the KCNE1 (minimal K(+)-channel protein) gene in the heart to form a cardiac-delayed rectifier-like K(+) current. Mutations in this channel can cause one form of inherited long QT syndrome type 1 (LQT1), as well as being associated with a form of deafness (Robbins Pharmacol Ther 2001, 90, 1-19).

The genes KCNQ2 and KCNQ3 were discovered in 1988 and appear to be mutated in an inherited form of epilepsy known as benign familial neonatal convulsions (Rogawski Trends in Neurosciences 2000, 23, 393-398). The proteins encoded by the KCNQ2 and KCNQ3 genes are localised in the pyramidal neurons of the human cortex and hippocampus, regions of the brain associated with seizure generation and propagation (Cooper et al. Proceedings National Academy of Science USA 2000, 97, 4914-4919).

KCNQ2 and KCNQ3 are two potassium channel subunits that form “M-currents” when expressed in vitro. The M-current is a non-inactivating potassium current found in many neuronal cell types. In each cell type, it is dominant in controlling membrane excitability by being the only sustained current in the range of action potential initiation (Marrion Annual Review Physiology 1997, 59, 483-504). Modulation of the M-current has dramatic effects on neuronal excitability, for example activation of the current will reduce neuronal excitability. Openers of these KCNQ channels, or activators of the M-current, will reduce excessive neuronal activity and may thus be of use in the treatment, prevention or inhibition of seizures and other diseases and disorders characterised by excessive neuronal activity, such as neuronal hyperexcitability including convulsive disorders, epilepsy and neuropathic pain.

Retigabine (D-23129; N-(2-amino-4-(4-fluorobenzylamino)-phenyl)carbamic acid ethyl ester) and analogues thereof are disclosed in EP554543. Retigabine is an anti-convulsive compound with a broad spectrum and potent anticonvulsant properties, both in vitro and in vivo. It is active after oral and intraperitoneal administration in rats and mice in a range of anticonvulsant tests including: electrically induced seizures, seizures induced chemically by pentylenetetrazole, picrotoxin and N-methyl-D-aspartate (NMDA) and in a genetic animal model, the DBA/2 mouse (Rostock et al. Epilepsy Research 1996, 23, 211-223). In addition, retigabine is active in the amygdala kindling model of complex partial seizures, further indicating that this compound has potential for anti-convulsive therapy. In clinical trials, retigabine has recently shown effectiveness in reducing the incidence of seizures in epileptic patients (Bialer et al. Epilepsy Research 2002, 51, 31-71).

Retigabine has been shown to activate a K(+) current in neuronal cells and the pharmacology of this induced current displays concordance with the published pharmacology of the M-channel, which recently was correlated to the KCNQ2/3 K(+) channel heteromultimer. This suggests that activation of KCNQ2/3 channels may be responsible for some of the anticonvulsant activity of this agent (Wickenden et al. Molecular Pharmacology 2000, 58, 591-600)—and that other agents working by the same mechanism may have similar uses.

KCNQ 2 and 3 channels have also been reported to be upregulated in models of neuropathic pain (Wickenden et al. Society for Neuroscience Abstracts 2002, 454.7), and potassium channel modulators have been hypothesised to be active in both neuropathic pain and epilepsy (Schroder et al. Neuropharmacology 2001, 40, 888-898).

Retigabine has also been shown to be beneficial in animal models of neuropathic pain (Blackburn-Munro and Jensen European Journal of Pharmacology 2003, 460, 109-116), and it is thus suggested that openers of KCNQ channels will be of use in treating pain disorders including neuropathic pain.

The localisation of KCNQ channel mRNA is reported in brain and other central nervous system areas associated with pain (Goldstein et al. Society for Neuroscience Abstracts 2003, 53.8).

In addition to a role in neuropathic pain, the expression of mRNA for KCNQ 2-5 in the trigeminal and dorsal root ganglia and in the trigeminal nucleus caudalis implies that openers of these channels may also affect the sensory processing of migraine pain (Goldstein et al. Society for Neuroscience Abstracts 2003, 53.8).

Recent reports demonstrate that mRNA for KCNQ 3 and 5, in addition to that for KCNQ2, are expressed in astrocytes and glial cells. Thus KCNQ 2, 3 and 5 channels may help modulate synaptic activity in the CNS and contribute to the neuroprotective effects of KCNQ channel openers (Noda et al., Society for Neuroscience Abstracts 2003, 53.9).

Retigabine and other KCNQ modulators may thus exhibit protection against the neurodegenerative aspects of epilepsy, as retigabine has been shown to prevent limbic neurodegeneration and the expression of markers of apoptosis following kainic acid-induced status epilepticus in the rat (Ebert et al. Epilepsia 2002, 43 Suppl 5, 86-95). This may have relevance for preventing the progression of epilepsy in patients, i.e. be anti-epileptogenic. Retigabine has also been shown to delay the progression of hippocampal kindling in the rat, a further model of epilepsy development (Tober et al. European Journal Of Pharmacology 1996, 303, 163-169).

It is thus suggested that these properties of retigabine and other KCNQ modulators may prevent neuronal damage induced by excessive neuronal activation, and may be of use in the treatment of neurodegenerative diseases, and be disease modifying (or antiepileptogenic) in patients with epilepsy.

Given that anticonvulsant compounds such as benzodiazepines and chlormethiazole are used clincially in the treatment of the ethanol withdrawal syndrome and that other anticonvulsant compounds e.g. gabapentin, are very effective in animal models of this syndrome (Watson et al. Neuropharmacology 1997, 36, 1369-1375), other anticonvulsant compounds such as KCNQ openers are thus expected to be effective in this condition.

mRNA for KCNQ 2 and 3 subunits are found in brain regions associated with anxiety and emotional behaviours such as bipolar disorder e.g. hippocampus and amygdala (Saganich et al. Journal of Neuroscience 2001, 21, 4609-4624), and retigabine is reportedly active in some animal models of anxiety-like behaviour (Hartz et al. Journal of Psychopharmacology 2003, 17 suppl 3, A28,B16), and other clinically used anticonvulsant compounds are used in the treatment of bipolar disorder.

WO 200196540 discloses the use of modulators of the M-current formed by expression of KCNQ2 and KCNQ3 genes for insomnia, while WO 2001092526 discloses that modulators of KCNQ5 can be utilized for the treatment of sleep disorders.

WO01/022953 describes the use of retigabine for prophylaxis and treatment of neuropathic pain such as allodynia, hyperalgesic pain, phantom pain, neuropathic pain related to diabetic neuropathy and neuropathic pain related to migraine.

WO02/049628 describes the use of retigabine for the prevention, treatment, inhibition and amelioration of anxiety disorders such as anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia and specific phobias.

WO97/15300 describes the use of retigabine for the treatment of neurodegenerative disorders such as Alzheimer's disease; Huntington's chorea; sclerosis such as multiple sclerosis and amyotrophic lateral sclerosis; Creutzfeld-Jakob disease; Parkinson's disease; encephalopathies induced by AIDS or infection by rubella viruses, herpes viruses, borrelia and unknown pathogens; trauma-induced neurodegenerations; neuronal hyperexcitation states such as in medicament withdrawal or intoxication; and neurodegenerative diseases of the peripheral nervous system such as polyneuropathies and polyneuritides.

Hence, there is a great desire for novel compounds, which are potent openers of the KCNQ family potassium channels.

Also desired are novel compounds with improved properties relative to known compounds, which are openers of the KCNQ family potassium channels, such as retigabine. Improvement of one or more of the following parameters is desired: half-life, clearance, selectivity, interactions with other medications, bioavailability, potency, formulability, chemical stability, metabolic stability, membrane permeability, solubility and therapeutic index. The improvement of such parameters may lead to improvements such as:
an improved dosing regime by reducing the number of required doses a day,
ease of administration to patients on multiple medications,
reduced side effects, enlarged therapeutic index,
improved tolerability or
improved compliance.

### SUMMARY OF THE INVENTION

One object of the present invention is to provide novel compounds, which are potent openers of the KCNQ family potassium channels.

The compounds of the invention are substituted indoline and indole derivatives of the general formula I or salts thereof 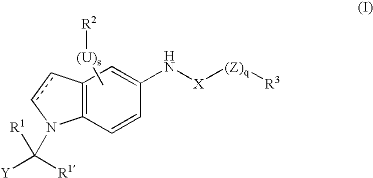
wherein the dotted line, q, s, U, Y, X, Z, R¹, R^{1′}, R² and R³ are as defined below.

The invention further relates to a pharmaceutical composition comprising a compound of formula I, and the use thereof.

### DESCRIPTION OF THE INVENTION

Accordingly, the present invention relates to substituted indole and indoline derivatives of the general formula I 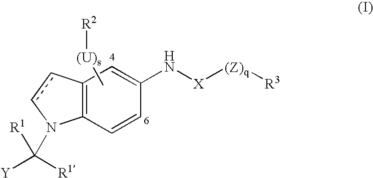
wherein
the dotted line represents an optional bond;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆alk(en/yn)yl, cyano-C₃₋₈-cycloalk(en)yl and cyano-C₃₋₈-cycloalk(en)yl-C₁₋₆-alk(en/yn)yl; or
R¹ and R^{1′} together with the carbon atom to which they are attached form a 3-8 membered saturated or unsaturated ring which optionally contains 1 or 2 heteroatoms;
s is 0 or 1;
U is O, NR¹¹, S, SO₂, SO₂NR¹¹, CO—O or CO—NR¹¹; wherein R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or R² and R¹¹ together with the nitrogen atom to which they are attached form a 4-8 membered saturated or unsaturated ring which optionally contains 1, 2 or 3 further heteroatoms;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹⁰ and R^{10′} together with the nitrogen atom to which they are attached form a 4-8 membered saturated or unsaturated ring which optionally contains 1, 2 or 3 further heteroatoms;
with the proviso that when R² is NO₂, halogen or cyano then s is 0; and
with the proviso that when R² is a hydrogen atom or acyl and s is 1 then U is NR¹¹, O or S;
wherein the group —(U)ₛ—R² is linked to position 4 or 6 of the indole or indoline;
q is 0 or 1;
Z is O or S;
X is CO or SO₂; with the proviso that q is 0 when X is SO₂;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆ alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl and —NR¹²R^{12′}, optionally substituted NR¹²R^{12′}—C₁₋₆-alk(en/yn)yl, optionally substituted NR¹²R^{12′}—C₃₋₈-cycloalk(en)yl, optionally substituted NR¹²R^{12′}—C₃₋₈-cycloalk(en)yl-C₁₋₆-alk(en/yn)yl; wherein
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹² and R^{12′} together with the nitrogen atom to which they are attached form a 4-8 membered saturated or unsaturated ring which optionally contains 1, 2 or 3 further heteroatoms;
with the proviso that when R³ is NR¹²R^{12′} then q is 0;
and
Y represents a group of formula II, III, IV, V, VI, XXX and XXX: 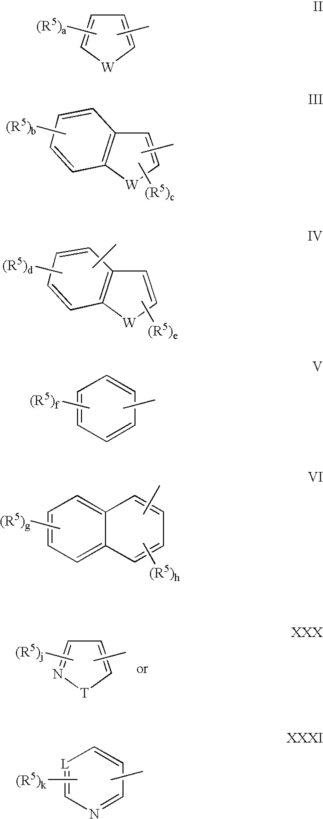
wherein
the line represents a bond attaching the group represented by Y to the carbon atom;
W is O or S;
T is N, NH or O;
L is N, C or CH;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1, 2 or 3; with the proviso that when T is a nitrogen atom then j is 0, 1, 2 or 3; and when T is NH or an oxygen atom then j is 0, 1 or 2;
k is 0, 1, 2, 3 or 4; and
each R⁵ is independently selected from the group consisting of a C₁₋₄-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸, or
two adjacent R⁵ together with the aromatic group to which they are attached form a 4-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl;
and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′} then R⁵ is not —S—R⁸;
or salts thereof.

A particular embodiment of the invention relates to substituted indole and indoline derivatives of the general formula I 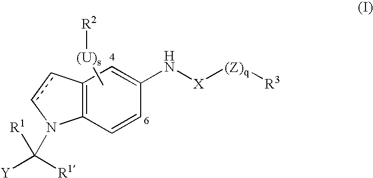
wherein
the dotted line represents an optional bond;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹ and R^{1′} form a 3-8 membered saturated or unsaturated ring which optionally contains 1 or 2 further heteroatoms;
s is 0 or 1;
U is O, NR¹¹, S, SO₂, SO₂NR¹¹, CO—O or CO—NR¹¹; wherein R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or
R² and R¹¹ together with the nitrogen atom to which they are attached form a 48 membered saturated or unsaturated ring which optionally contains 1, 2 or 3 further heteroatoms;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹⁰ and R^{10′} together with the nitrogen atom to which they are attached form a 48 membered saturated or unsaturated ring which optionally contains 1, 2 or 3 further heteroatoms;
with the proviso that when R² is NO₂, halogen or cyano then s is 0; and
with the proviso that when R² is a hydrogen atom or acyl and s is 1 then U is NR¹¹, O or S;
wherein the group —(U)ₛ—R² is linked to position 4 or 6 of the indole or indoline;
q is 0 or 1;
Z is O or S;
X is CO or SO₂; with the proviso that q is 0 when X is SO₂;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₉-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₉-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl and —NR¹²R^{12′}; wherein
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹² and R^{12′} together with the nitrogen atom to which they are attached form a 4-8 membered saturated or unsaturated ring which optionally contains 1, 2 or 3 further heteroatoms;
with the proviso that when R³ is NR¹²R^{12′} then q is 0;
and
Y represents a group of formula II, III, IV, V and VI: 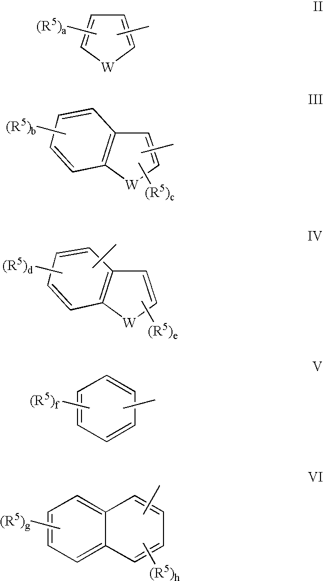
wherein
the line represents a bond attaching the group represented by Y to the carbon atom;
W is O or S;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3; and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸, or
two adjacent R⁵ together with the aromatic group to which they are attached form a 4-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl;
and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′} then R⁵ is not —S—R⁸;
or salts thereof.

One embodiment of the invention relates to compounds of formula I, wherein the dotted line represents a bond.

Another embodiment of the invention relates to compounds of formula I, wherein the dotted line does not represent a bond.

One further embodiment of the invention relates to compounds of formula I, wherein R¹ and R^{1′} are independently selected from the group consisting of hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl.

Another embodiment of the invention relates to compounds of formula I, wherein and R¹ are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl.

A further embodiment of the invention relates to compounds of formula I, wherein R¹ and R^{1′} form a 3-8 membered saturated or unsaturated ring which optionally contains 1 or 2 heteroatoms. In a further embodiment the 3-8 membered saturated or unsaturated ring is a saturated carbocyclic ring, typically cyclopropyl, cyclobutyl, cyclopentyl, or cyclohexyl.

Yet another embodiment of the invention relates to compounds of formula I, wherein R¹ and R^{1′} are independently selected from the group consisting of hydrogen and C₁₋₆-alk(en/yn)yl.

In yet another embodiment, the invention relates to compounds of formula I, wherein at least one of R¹ and R^{1′} is C₁₋₆-alk(en/yn)yl, typically C₁₋₃-alk(en/yn)yl.

In a preferred embodiment, the invention relates to compounds of formula I, wherein R¹ or R^{1′} is a hydrogen atom.

In a preferred embodiment, the invention relates to compounds of formula I, wherein at least one of R¹ and R^{1′} is a hydrogen atom.

In a more preferred embodiment, the invention relates to compounds of formula I, wherein both R¹ and R^{1′} are hydrogen atoms.

In a preferred embodiment, the invention relates to compounds of formula I, wherein s is 0.

In another preferred embodiment, the invention relates to compounds of formula I, wherein s is 1.

In one embodiment, the invention relates to compounds of formula I, wherein s is 1 and U is O.

In another embodiment, the invention relates to compounds of formula I, wherein s is 1 and U is S.

In yet another embodiment, the invention relates to compounds of formula I, wherein s is 1 and U is SO₂.

In yet another embodiment, the invention relates to compounds of formula I, wherein s is 1 and U is SO₂NR¹¹.

In yet another embodiment, the invention relates to compounds of formula I, wherein s is 1 and U is CO—O.

In yet another embodiment, the invention relates to compounds of formula I, wherein s is 1 and U is CO—NR¹¹.

In a preferred embodiment, the invention relates to compounds of formula I, wherein s is 1 and U is NR¹¹.

In yet another embodiment, the invention relates to compounds of formula I, wherein s is 1 and U is SO₂NR¹¹, CO—NR¹¹ or NR¹¹ and R¹¹ is a hydrogen atom.

In a preferred embodiment, the invention relates to compounds of formula I, wherein s is 1 and U is NR¹¹ and R¹¹ is a hydrogen atom.

One embodiment of the invention relates to compounds of formula I, wherein R² is selected from the group consisting of acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, cyano-C₁₋₆-alk(en/yn)yl, cyano-C₃₋₈cycloalk(en)yl, cyano-C₃₋₈-cycloalk(en)yl-C₁₋₆-alk(en/yn)yl, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl;

with the proviso that when R² is acyl and s is 1 then U is NR¹¹, O or S.

Another embodiment of the invention relates to compounds of formula I, wherein R² is selected from the group consisting of C₁₋₆-alk(en/yn)yl, C₃₋₈-cycloalk(en)yl and C₃₋₈-cycloalk(en)yl-C₁₋₆-alk(en/yn)yl.

Yet another embodiment of the invention relates to compounds of formula I, wherein R² is selected from the group consisting of Ar, Ar—C₁₋₆-alk(en/yn)yl, Ar—C₃₋₈-cycloalk(en)yl and Ar—C₃₋₈-cycloalk(en)yl-C₁₋₆-alk(en/yn)yl.

Yet another embodiment of the invention relates to compounds of formula I, wherein R² is selected from the group consisting of halogen, halo-C₁₋₆-alk(en/yn)yl, halo-C₃₋₈-cycloalk(en)yl, halo-C₃₋₈-cycloalk(en)yl-C₁₋₆-alk(en/yn)yl and cyano;

with the proviso that when R² is halogen or cyano then s is 0.

In a preferred embodiment, the invention relates to compounds of formula I, wherein R² is NO₂ or a hydrogen atom;

with the proviso that when R² is NO₂ then s is 0; and

with the proviso that when R² is a hydrogen atom and s is 1 then U is NR¹¹, O or S.

In one embodiment, the invention relates to compounds of formula I, wherein R² is NO₂ or a hydrogen atom or a halogen atom;

with the proviso that when R² is NO₂ or a halogen atom then s is 0; and

with the proviso that when R² is a hydrogen atom and s is 1 then U is NR¹¹, O or S.

In another embodiment, the invention relates to compounds of formula I, wherein s is 0 and R² is NO₂ or a halogen atom.

In one embodiment, the invention relates to compounds of formula I, wherein R² is a hydrogen atom.

In one embodiment, the invention relates to compounds of formula I, wherein s is 0 and R² is selected from the group consisting of NO₂, halogen and cyano.

In another embodiment, the invention relates to compounds of formula I, wherein s is 0 and R² is a hydrogen atom.

In yet another embodiment, the invention relates to compounds of formula I, wherein R² is C₁₋₆-alk(en/yn)yl, typically C₁₋₃-alk(en/yn)yl.

In yet another embodiment, the invention relates to compounds of formula I, wherein R² is C₃₋₈-cycloalk(en)yl, typically C₃₋₆-cycloalk(en)yl.

In yet another embodiment, the invention relates to compounds of formula I, wherein R² is Ar.

In yet another embodiment, the invention relates to compounds of formula I, wherein R² is Ar—C₁₋₆-alk(en/yn)yl, typically Ar—C₁₋₃-alk(en/yn)yl.

In yet another embodiment, the invention relates to compounds of formula I, wherein R² is halo-C₁₋₆-alk(en/yn)yl, typically halo-C₁₋₃-alk(en/yn)yl.

In yet another embodiment, the invention relates to compounds of formula I, wherein s is 0 and R² is a halogen atom.

In yet another embodiment, the invention relates to compounds of formula I, wherein s is 0 and R² is cyano.

In another preferred embodiment, the invention relates to compounds of formula I, wherein s is 0 and R² is NO₂.

In a preferred embodiment, the invention relates to compounds of formula I, wherein R² is a hydrogen atom; with the proviso that when s is 1 then U is NR¹¹, O or S.

In one embodiment, the invention relates to compounds of formula I, wherein R² is a hydrogen atom; with the proviso that when s is 1 then U is NR¹¹.

In another embodiment, the invention relates to compounds of formula I, wherein R² is a hydrogen atom, s is 1, U is NR¹¹ and R¹¹ is a hydrogen atom.

In one embodiment, the invention relates to compounds of formula I, wherein the group —(U)ₛ—R² is linked to position 6 of the indole or indoline.

In a preferred embodiment, the invention relates to compounds of formula I, wherein the group —(U)ₛ—R² is linked to position 4 of the indole or indoline.

In a preferred embodiment, the invention relates to compounds of formula I, wherein X is CO.

In a preferred embodiment, the invention relates to compounds of formula I, wherein X is SO₂.

In a preferred embodiment, the invention relates to compounds of formula I, wherein q is 0.

In a preferred embodiment, the invention relates to compounds of formula I, wherein q is 1.

In one embodiment, the invention relates to compounds of formula I, wherein q is 1 and Z is a sulphur atom.

In a preferred embodiment, the invention relates to compounds of formula I, wherein q is 1 and Z is an oxygen atom.

In one embodiment, the invention relates to compounds of formula I, wherein X is SO₂ and q is 0.

In one embodiment, the invention relates to compounds of formula I, wherein X is CO and q is 0.

In one embodiment, the invention relates to compounds of formula I, wherein X is CO, q is 1 and Z is an oxygen atom.

In one embodiment, the invention relates to compounds of formula I, wherein R³ is selected from the group consisting of C₁₋₆-alk(en/yn)yl-heterocycloalk(en)yl, Ar-heterocycloalk(en)yl, Ar—C₁₋₆-alk(en/yn)yl-heterocycloalk(en)yl, C₁₋₆-alk(en/yn)yloxy-heterocycloalk(en)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-heterocycloalk(en)yl, halo-C₁₋₆-alk(en/yn)yl-heterocycloalk(en)yl, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl and acyl-C₁₋₆-alk(en/yn)yl-heterocycloalk(en)yl.

In another embodiment, the invention relates to compounds of formula I, wherein R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, Ar, Ar—C₁₋₆-alk(en/yn)yl, Ar—C₃₋₈-cycloalk(en)yl, Ar—C₃₋₄-cycloalk(en)yl-C₁₋₆-alk(en/yn)yl, Ar—C₁₋₆-alk(en/yn)yl-C₃₋₈-cycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₁₆-alk(en/yn)yloxy-C₃₋₈-cycloalk(en)yl, Ar-oxy-C₁₋₆-alk(en/yn)yl, Ar—C₁₋₆-alk(en/yn)yloxy-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar and —NR¹²R^{12′};

with the proviso that when R³ is NR¹²R^{12′} then q is 0.

In yet another embodiment, the invention relates to compounds of formula I, wherein R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar-oxy-C₁₋₆-alk(en/yn)yl, Ar—C₁₋₆-alk(en/yn)yloxy-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl and —NR¹²R^{12′}; with the proviso that when R³ is NR¹²R^{12′} then q is 0.

In yet another embodiment, the invention relates to compounds of formula I, wherein R³ is selected from the group consisting of C₁₋₆-alk(en/yn)yl, C₃₋₈-cycloalk(en)yl, C₃₋₈-cycloalk(en)yl-C₁₋₆-alk(en/yn)yl, C₁₋₆-alk(en/yn)yl-C₃₋₈-cycloalk(en)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar-oxy-C₁₋₆-alk(en/yn)yl, Ar—C₁₋₆-alk(en/yn)yloxy-C₁₋₆-alk(en/yn)yl and —NR¹²R^{12′};

with the proviso that when R³ is NR¹²R^{12′} then q is 0.

In a preferred embodiment, the invention relates to compounds of formula I, wherein R³ is selected from the group consisting of C₁₋₆-alk(en/yn)yl, C₃₋₈-cycloalk(en)yl, heterocycloalk(en)yl, Ar, Ar—C₁₋₆-alk(en/yn)yl, Ar-oxy-C₁₋₆-alk(en/yn)yl, Ar—C₁₋₆-alk(en/yn)yloxy-C₁₋₆-alk(en/yn)yl, halo-C₁₋₆-alk(en/yn)yl and —NR¹²R^{12′};

with the proviso that when R³ is NR¹²R^{12′} then q is 0.

In another preferred embodiment, the invention relates to compounds of formula I, wherein R³ is selected from the group consisting of C₁₋₆-alk(en/yn)yl, C₃₋₈-cycloalk(en)yl, Ar, Ar—C₁₋₆-alk(en/yn)yl, Ar-oxy-C₁₋₆-alk(en/yn)yl, Ar—C₁₋₆-alk(en/yn)yloxy-C₁₋₆-alk(en/yn)yl and —NR¹²R^{12′};

with the proviso that when R³ is NR¹²R^{12′} then q is 0.

In another preferred embodiment, the invention relates to compounds of formula I, wherein R³ is C₁₋₆-alk(en/yn)yl, typically C₁₋₃-alk(en/yn)yl.

In yet another preferred embodiment, the invention relates to compounds of formula I, wherein R³ is C₃₋₈-cycloalk(en)yl.

In yet another preferred embodiment, the invention relates to compounds of formula I, wherein R³ is C₃₋₈-cycloalk(en)yl-C₁₋₆-alk(en/yn)yl.

In yet another preferred embodiment, the invention relates to compounds of formula I, wherein R³ is heterocycloalk(en)yl.

In yet another preferred embodiment, the invention relates to compounds of formula I, wherein R³ is Ar.

In yet another preferred embodiment, the invention relates to compounds of formula I, wherein R³ is Ar—C₁₋₆-alk(en/yn)yl.

In yet another preferred embodiment, the invention relates to compounds of formula I, wherein R³ is C₁₋₆-alk(en/yn)yl-oxy-C₁₋₆-alk(en/yn)yl.

In yet another preferred embodiment, the invention relates to compounds of formula I, wherein R³ is Ar-oxy-C₁₋₆-alk(en/yn)yl.

In yet another preferred embodiment, the invention relates to compounds of formula I, wherein R³ is Ar—C₁₋₆-alk(en/yn)yloxy-C₁₋₆-alk(en/yn)yl.

In yet another embodiment, the invention relates to compounds of formula I, wherein R³ is C₁₋₆-alk(en/yn)yloxy-carbonyl-C₁₋₆-alk(en/yn)yl.

In yet another embodiment, the invention relates to compounds of formula I, wherein R³ is halo-C₁₋₆-alk(en/yn)yl, such as halo-C₁₋₃-alk(en/yn)yl.

In yet another embodiment, the invention relates to compounds of formula I, wherein R³ is halo-C₁₋₆-alk(en/yn)yl-Ar, such as halo-C₁₋₃-alk(en/yn)yl-Ar.

In yet another preferred embodiment, the invention relates to compounds of formula I, wherein R³ is —NR¹²R^{12′}, and q is 0.

In one embodiment, the invention relates to compounds of formula I, wherein X is CO, q is 1, Z is an oxygen atom and R³ is selected from the group consisting of C₁₋₆-alk(en/yn)yl, C₃₋₈-cycloalk(en)yl, C₃₋₈-cycloalk(en)yl-C₁₋₆-alk(en/yn)yl, C₁₋₆-alk(en/yn)yl-C₃₋₈-cycloalk(en)yl, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar-oxy-C₁₋₆-alk(en/yn)yl, Ar—C₁₋₆-alk(en/yn)yloxy-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl and halo-C₁₋₆-alk(en/yn)yl-C₃₋₈-cycloalk(en)yl.

In another embodiment, the invention relates to compounds of formula I, wherein X is CO, q is 1, Z is an oxygen atom and R³ is selected from the group consisting of C₁₋₆-alk(en/yn)yl, Ar—C₁₋₆-alk(en/yn)yloxy-C₁₋₆-alk(en/yn)yl and halo-C₁₋₆-alk(en/yn)yl.

In one further embodiment, the invention relates to compounds of formula I, wherein X is CO, q is 0 and R³ is selected from the group consisting of C₁₋₆-alk(en/yn)yl, C₃₋₈-cycloalk(en)yl, C₃₋₈-cycloalk(en)yl-C₁₋₆-alk(en/yn)yl, C₁₋₆-alk(en/yn)yl-C₃₋₈-cycloalk(en)yl, heterocycloalk(en)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar-oxy-C₁₋₆-alk(en/yn)yl, Ar—C₁₋₆-alk(en/yn)yloxy-C₁₋₆-alk(en/yn)yl and —NR¹²R^{12′}.

In yet another embodiment, the invention relates to compounds of formula I, wherein X is CO, q is 0 and R³ is selected from the group consisting of C₁₋₆-alk(en/yn)yl, C₃₋₈-cycloalk(en)yl, heterocycloalk(en)yl, Ar, Ar—C₁₋₆-alk(en/yn)yl, Ar-oxy-C₁₋₆-alk(en/yn)yl and —NR¹²R^{12′}.

In yet another embodiment, the invention relates to compounds of formula I, wherein X is SO₂, q is 0 and R³ is selected from the group consisting of C₁₋₆-alk(en/yn)yl, C₃₋₈-cycloalk(en)yl, C₃₋₈-cycloalk(en)yl-C₁₋₆-alk(en/yn)yl, C₁₋₆-alk(en/yn)yl-C₃₋₈-cycloalk(en)yl, Ar—C₁₋₆-alk(en/yn)yl, Ar—C₃₋₈-cycloalk(en)yl, Ar—C₃₋₈-cycloalk(en)yl-C₁₋₆-alk(en/yn)yl and Ar—C₁₋₆-alk(en/yn)yl-C₃₋₈-cycloalk(en)yl.

In yet another embodiment, the invention relates to compounds of formula I, wherein X is SO₂, q is 0 and R³ is C₁₋₆-alk(en/yn)yl or Ar—C₁₋₆-alk(en/yn)yl.

In one embodiment, the invention relates to compounds of formula I, wherein R³ is NR¹²R^{12′} and q is 0 and wherein R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl and Ar—C₃₋₈-cycloalk(en)yl-C₁₋₆-alk(en/yn)yl.

In another embodiment, the invention relates to compounds of formula I, wherein R³ is NR¹²R^{12′} and q is 0 and wherein R¹² and R^{12′} are independently selected from the group consisting of hydrogen, C₁₋₆-alk(en/yn)yl, Ar and Ar—C₁₋₆-alk(en/yn)yl or wherein R¹² and R^{12′} together with the nitrogen atom to which they are attached form a 48 membered saturated or unsaturated ring which optionally contains 1, 2 or 3 further heteroatoms.

In a preferred embodiment, the invention relates to compounds of formula I, wherein R³ is NR¹²R^{12′} and q is 0 and wherein R¹² and R^{12′} are independently selected from the group consisting of hydrogen, C₁₋₆-alk(en/yn)yl, Ar and Ar—C₁₋₆-alk(en/yn)yl.

In another preferred embodiment, the invention relates to compounds of formula I, wherein R³ is NR¹²R^{12′} and q is 0 and wherein R¹² and R^{12′} together with the nitrogen atom to which they are attached form a 4-8 membered saturated or unsaturated ring which optionally contains 1, 2 or 3 further heteroatoms.

In one embodiment, the invention relates to compounds of formula I, wherein R³ is NR¹²R^{12′} and q is 0 and wherein at least one of R¹² and R^{12′} is a hydrogen atom.

In another embodiment, the invention relates to compounds of formula I, wherein R³ is NR¹²R^{12′} and q is 0 and at least one of R¹² and R^{12′} is C₁₋₆-alk(en/yn)yl, typically C₁₋₃-alk(en/yn)yl.

In yet another embodiment, the invention relates to compounds of formula I, wherein R³ is NR¹²R^{12′} and q is 0 and one of R¹² and R^{12′} is Ar.

In yet another embodiment, the invention relates to compounds of formula I, wherein R³ is NR¹²R^{12′} and q is 0 and one of R¹² and R^{12′} is Ar—C₁₋₆-alk(en/yn)yl, typically Ar—C₁₋₃-alk(en/yn)yl.

In a preferred embodiment, the invention relates to compounds of formula I, wherein Y is of formula II, III, V, XXX or XXXI.

In one embodiment, the invention relates to compounds of formula I, wherein Y is of formula III or IV.

In a preferred embodiment, the invention relates to compounds of formula I, wherein Y is of formula II or V.

In a preferred embodiment, the invention relates to compounds of formula I, wherein Y is of formula V or XXXI.

In a preferred embodiment, the invention relates to compounds of formula I, wherein Y is of formula II or III and W is a sulphur atom.

In a preferred embodiment, the invention relates to compounds of formula I, wherein Y is of formula II or III and W is an oxygen atom.

In a preferred embodiment, the invention relates to compounds of formula I, wherein Y is of formula V.

In a preferred embodiment, the invention relates to compounds of formula I, wherein Y is of formula XXX and T is NH.

In a preferred embodiment, the invention relates to compounds of formula I, wherein Y is of formula XXX and T is a nitrogen atom or an oxygen atom.

In a preferred embodiment, the invention relates to compounds of formula I, wherein Y is of formula XXXI and L is a nitrogen atom.

In a preferred embodiment, the invention relates to compounds of formula I, wherein Y is of formula XXXI and L is C or CH.

In one embodiment, the invention relates to compounds of formula I, wherein each R⁵ is independently selected from the group consisting of Ar—C₁₋₆-alk(en/yn)yl, acyl, —CO—NR⁶R^{6′}, cyano, cyano-C₁₋₆-alk(en/yn)yl, cyano-C₃₋₈-cycloalk(en)yl and cyano-C₃₋₈-cycloalk(en)yl-C₁₋₆-alk(en/yn)yl.

In another embodiment, the invention relates to compounds of formula I, wherein each R⁵ is independently selected from the group consisting of C₁₋₆-alk(en/yn)yl, C₃₋₈-cycloalk(en)yl, C₃₋₈-cycloalk(en)yl-C₁₋₆-alk(en/yn)yl, Ar, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —NR⁷R^{7′}, —S—R⁸ and —SO₂R⁸; or

two adjacent R⁵ together with the aromatic group to which they are attached form a 4-8 membered ring which optionally contains one or two heteroatoms.

In yet another embodiment, the invention relates to compounds of formula I, wherein each R⁵ is independently selected from the group consisting of C₁₋₆-alk(en/yn)yl, C₃₋₈-cycloalk(en)yl, C₃₋₈-cycloalk(en)yl-C₁₋₆-alk(en/yn)yl, Ar, C₁₋₆-alk(en/yn)yloxy, C₃₋₈-cycloalk(en)yloxy, C₃₋₈-cycloalk(en)yl-C₁₋₆-alk(en/yn)yloxy, —NR⁷R^{7′}, —S—R⁸ and —SO₂R⁸; or

two adjacent R⁵ together with the aromatic group to which they are attached form a 4-8 membered ring which optionally contains one or two heteroatoms.

In yet another embodiment, the invention relates to compounds of formula I, wherein each R⁵ is independently selected from the group consisting of halogen, halo-C₁₋₆-alk(en/yn)yl, halo-C₃₋₈-cycloalk(en)yl and halo-C₃₋₈-cycloalk(en)yl-C₁₋₆-alk(en/yn)yl.

In yet another embodiment, the invention relates to compounds of formula I, wherein each R⁵ is independently selected from the group consisting of C₁₋₆alk(en/yn)yl, C₃₋₈-cycloalk(en)yl, C₃₋₈-cycloalk(en)yl-C₁₋₆-alk(en/yn)yl, Ar, Ar-thio, Ar-oxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, or

two adjacent R⁵ together with the aromatic group to which they are attached form a 4-8 membered ring which optionally contains one or two heteroatoms.

In a preferred embodiment, the invention relates to compounds of formula I, wherein each R⁵ is independently selected from the group consisting of C₁₋₆-alk(en/yn)yl, Ar, Ar-thio, Ar-oxy, halogen, halo-C₁₋₆-alk(en/yn)yl, or

two adjacent R⁵ together with the aromatic group to which they are attached form a 4-8 membered ring which optionally contains one or two heteroatoms.

In another preferred embodiment, the invention relates to compounds of formula I, wherein each R⁵ is independently selected from the group consisting of halogen and halo-C₁₋₆-alk(en/yn)yl.

In an embodiment, the invention relates to compounds of formula I, wherein at least one substituent R⁵ is a halogen atom.

In another embodiment, the invention relates to compounds of formula I, wherein at least one substituent R⁵ is halo-C₁₋₆-alk(en/yn)yl, typically halo-C₁₋₃-alk(en/yn)yl.

In yet another embodiment, the invention relates to compounds of formula I, wherein at least one substituent R⁵ is C₁₋₆-alk(en/yn)yl.

In yet another embodiment, the invention relates to compounds of formula I, wherein at least one substituent R⁵ is Ar.

In yet another embodiment, the invention relates to compounds of formula I, wherein at least one substituent R⁵ is Ar-thio.

In yet another embodiment, the invention relates to compounds of formula I, wherein at least one substituent R⁵ is Ar-oxy.

In yet another embodiment, the invention relates to compounds of formula I, wherein at least one substituent R⁵ is C₁₋₆-alk(en/yn)yloxy.

In yet another embodiment, the invention relates to compounds of formula I, wherein at least one substituent R⁵ is —NR⁷R^{7′}.

In yet another embodiment, the invention relates to compounds of formula I, wherein at least one substituent R⁵ is —S—R⁸.

In yet another embodiment, the invention relates to compounds of formula I, wherein at least one substituent R⁵ is —SO₂R⁸.

In yet another embodiment, the invention relates to compounds of formula I, wherein two adjacent R⁵ together with the aromatic group form a 4-8 membered ring, which optionally contains one or two heteroatoms.

In a preferred embodiment, the invention relates to compounds of formula I, wherein two adjacent R⁵ together form

—(CH₂)_{n′}—CH₂—, —CH═CH—(CH₂)_{m′}—, —CH₂—CH═CH—(CH₂)_{p′}—, —CH═CH—CH═CH—, —(CH₂)_{n′}—O—, —O(CH₂)_{m′}—O—, —CH₂—O—(CH₂)_{p′}—O—, —CH₂—O—CH₂—O—CH₂—, —(CH₂)_{n′}—S—, —S—(CH₂)_{m′}—S—, —CH₂—S—(CH₂)_{p′}—S—, —CH₂—S—CH₂—S—CH₂—, —(CH₂)_{n′}—NH—, —NH—(CH₂)_{m′}—NH—, —CH₂—NH—(CH₂)_{p′}—NH—, —CH═CH—NH—, —O—(CH₂)_{m′}—NH—, —CH₂—O—(CH₂)_{p′}—NH— or —O—(CH₂)_{p′}—NH—CH₂—, —S—(CH₂)_{m′}—NH—, —N═CH—NH—, —N═CH—O— or —N═CH—S—, wherein m′ is 1, 2 or 3, n′ is 2, 3 or 4 and p′ is 1 or 2.

In yet another embodiment, the invention relates to compounds of formula I, wherein two adjacent R⁵ together form —CH₂—O—CH₂—.

In yet another embodiment, the invention relates to compounds of formula I, wherein two adjacent R⁵ together form —CH═CH—CH═CH—.

In yet another embodiment, the invention relates to compounds of formula I, wherein two adjacent R⁵ together form —CH₂—O—.

In yet another embodiment, the invention relates to compounds of formula I, wherein two adjacent R⁵ together form —O—CH₂—O—CH₂—.

In yet another embodiment, the invention relates to compounds of formula I, wherein at least one substituent R⁵ is —NR⁷R^{7′}; and wherein R⁷ and R^{7′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl.

In yet another embodiment, the invention relates to compounds of formula I, wherein at least one substituent R⁵ is —NR⁷R^{7′}; and wherein R⁷ and R^{7′} are independently selected from the group consisting of hydrogen and C₁₋₆-alk(en/yn)yl.

In yet another embodiment, the invention relates to compounds of formula I, wherein at least one substituent R⁵ is —NR⁷R^{7′}; and wherein both R⁷ and R^{7′} are C₁₋₉-alk(en/yn)yl, typically C₁₋₃-alk(en/yn)yl.

In yet another embodiment, the invention relates to compounds of formula I, wherein at least one substituent R⁵ is —S—R⁸ or —SO₂R⁸; and wherein R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar.

In yet another embodiment, the invention relates to compounds of formula I, wherein at least one substituent R⁵ is —S—R⁸ or —SO₂R⁸; and wherein R⁸ is selected from the group consisting of C₁₋₆-alk(en/yn)yl and Ar.

One embodiment of the invention relates to compounds of formula I, wherein s is 0 and q is 0.

Another embodiment of the invention relates to compounds of formula I, wherein R² is a hydrogen atom and X is CO.

Yet another embodiment of the invention relates to compounds of formula I, wherein s is 0 and X is CO.

Yet another embodiment of the invention relates to compounds of formula I, wherein R² is a hydrogen atom and q is 0.

Yet another embodiment of the invention relates to compounds of formula I, wherein q is 0 and X is CO.

One embodiment of the invention relates to compounds of formula I, wherein the total number of Ar-groups in the substituents R², R³ and R⁵ equals to 0, 1, 2, or 3, typically 0 or 1.

Another embodiment of the invention relates to compounds of formula I wherein neigther R², R³ or R⁵ comprises an Ar-group.

Yet another embodiment of the invention relates to compounds of formula I, wherein the total number of Ar-groups in the substituents R², R³ and R⁵ equals to 1.

Yet another embodiment of the invention relates to compounds of formula I, wherein the total number of Ar-groups in the substituents R¹, R³ and R⁵ equals to 2.

One embodiment of the invention relates to compounds of formula I, wherein R³ is not CH₃ when X is SO₂ and q is 0.

Another embodiment of the invention relates to compounds of formula I, wherein X-(Z)_{q}—R³ is not SO₂—CH₃ when Y is of formula V.

Yet another embodiment of the invention relates to compounds of formula I, wherein R³ is NR¹²R^{12′} and both R¹² and R^{12′} is different from Ar.

Yet another embodiment of the invention relates to compounds of formula I, wherein R³ is NR¹²R^{12′} and one of R¹² and R^{12′} is Ar, with the proviso that Ar is different from quinoline or phenyl.

Another embodiment of the invention relates to compounds of formula I, wherein Y is not of formula V when X is CO and q is 0 and R³ is NR¹²R^{12′} and one of R¹² and R^{12′} is Ar, typically quinoline or phenyl.

In another embodiment, the compound of formula I is not:
N-[1-(phenylmethyl)-1H-indol-5-yl]-Methanesulfonamide;
N-[1-[(4-fluorophenyl)methyl]-1H-indol-5-yl]-Methanesulfonamide;
N-[2,3-dihydro-1-phenylmethyl)-1H-indol-5-yl]-Methanesulfonamide;
N-[1-(phenylmethyl)-1H-indol-5-yl]-N′-4-quinolinyl-Urea;
N-[1-(phenylmethyl)-1H-indol-5-yl]-N′-4-quinolinyl-Urea; or
1-(1-benzyl-5-indolinyl)-3-phenyl-Urea.

One aspect of the invention, relates to compounds of general formula VII and salts thereof: 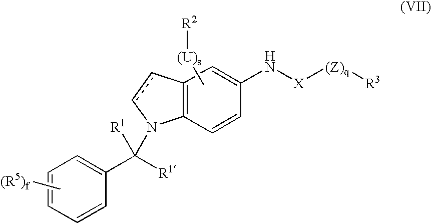
wherein the dotted line, f, q, s, U, X, Z, R¹, R^{1′}, R², R³ and R⁵ are as defined under formula I. Any of the embodiments related to formula I are also embodiments of formula VII.

In one embodiment, the invention relates to compounds of the general formula VII, wherein f is 0.

In another embodiment, the invention relates to compounds of the general formula VII being substituted by one substituent R⁵, such as in the orto-, meta- or para-position.

In a preferred embodiment, the invention relates to compounds of the general formula VII, which are substituted by one substituent R⁵ in the para-position.

In one embodiment, the invention relates to compounds of the general formula VII being substituted by two independently selected R⁵ substituents, such as in the ortho- and para-position, in the meta- and para-position and in the orto- and meta-position.

In another embodiment, the invention relates to compounds of the general formula VII being substituted by three independently selected R⁵ substituents.

Another aspect of the invention relates to compounds of the general formula VIII or salts thereof: 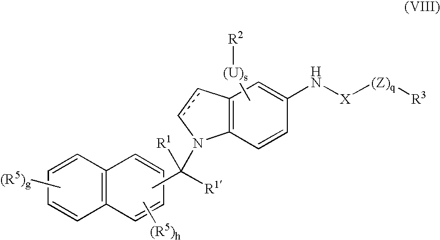
wherein the dotted line, g, h, q, s, U, X, Z, R¹, R^{1′}, R², R³ and R⁵ are as defined under formula I. Any of the embodiments related to formula I are also embodiments of formula VIII.

In one embodiment, the invention relates to compounds of the general formula VIII, wherein the nitrogen atom is attached to position 1 of the naphtyl group via the methylene group.

In another embodiment, the invention relates to compounds of the general formula VIII, wherein the nitrogen atom is attached to position 2 of the naphtyl group via the methylene group.

In yet another embodiment, the invention relates to compounds of the general formula VIII, wherein g is 0, 1, 2 or 3, typically 0, 1 or 2.

In yet another embodiment, the invention relates to compounds of the general formula VIII, wherein h is 0, 1 or 2, typically 0 or 1.

In yet another embodiment, the invention relates to compounds of the general formula VIII, wherein g+h equals to 0, 1, 2 or 3.

In yet another embodiment, the invention relates to compounds of the general formula VIII, wherein both g and h are 0.

In yet another embodiment, the invention relates to compounds of the general formula VIII being substituted by one substituent R⁵.

In yet another embodiment, the invention relates to compounds of the general formula VIII being substituted by two independently selected R⁵ substituents.

In yet another embodiment, the invention relates to compounds of the general formula VIII being substituted by three independently selected R⁵ substituents.

Yet another aspect of the invention relates to compounds of the general formula IX or salts thereof: 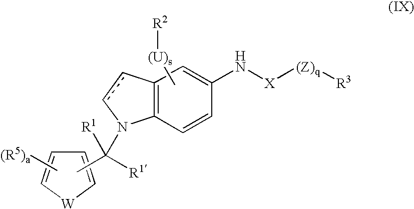
wherein the dotted line, a, q, s, U, X, Z, R¹, R^{1′}, R², R³ and R⁵ are as defined under formula I. Any of the embodiments related to formula I are also embodiments of formula IX.

In an embodiment, the invention relates to compounds of the general formula IX, wherein the nitrogen atom is attached to position 2 of the heteroaromatic group via the methylene group.

In another embodiment, the invention relates to compounds of the general formula IX, wherein the nitrogen atom is attached to position 3 of the heteroaromatic group via the methylene group.

In yet another embodiment, the invention relates to compounds of the general formula IX, wherein W is an oxygen atom.

In a preferred embodiment, the invention relates to compounds of the general formula IX, wherein W is a sulphur atom.

In another embodiment, the invention relates to compounds of the general formula IX, wherein a is 0, 1 or 2.

In yet another embodiment, the invention relates to compounds of the general formula IX, wherein a is 0.

In yet another embodiment, the invention relates to compounds of the general formula IX being substituted by one substituent R⁵, such as in position 5.

In yet another embodiment, the invention relates to compounds of the general formula IX being substituted by two independently selected R⁵ substituents.

In an embodiment, the invention relates to compounds of the general formula IX, wherein the nitrogen atom is attached to position 2 via the methylene group and wherein a substituent R⁵ is attached to position 5 of the heteroaromatic group.

Yet another aspect of the invention relates to compounds of the general formula X or salts thereof: 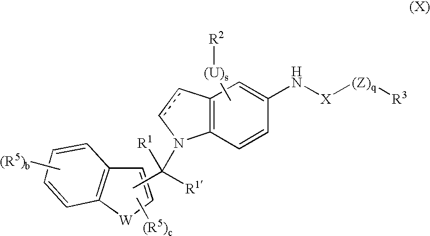
wherein the dotted line, b, c, q, s, U, X, Z, R¹, R^{1′}, R², R³ and R⁵ are as defined under formula I. Any of the embodiments related to formula I are also embodiments of formula X.

In an embodiment, the invention relates to compounds of the general formula X, wherein the nitrogen atom is attached to position 2 of the heteroaromatic group via the methylene group.

In another embodiment, the invention relates to compounds of the general formula X, wherein the nitrogen atom is attached to position 3 of the heteroaromatic group via the methylene group.

In yet another embodiment, the invention relates to compounds of the general formula X, wherein W is an oxygen atom.

In yet another embodiment, the invention relates to compounds of the general formula X, wherein W is a sulphur atom.

In yet another embodiment, the invention relates to compounds of the general formula X, wherein b is 0, 1, 2 or 3, typically 0, 1 or 2.

In yet another embodiment, the invention relates to compounds of the general formula X, wherein c is 0 or 1, typically 0.

In yet another embodiment, the invention relates to compounds of the general formula X, wherein b+c equals to 0, 1, 2, 3 or 4.

In yet another embodiment, the invention relates to compounds of the general formula X, wherein both b and c are 0.

In yet another embodiment, the invention relates to compounds of the general formula X wherein b+c equals to 1. In one aspect thereof b is 1 and c is 0. In another aspect thereof b is 0 and c is 1.

In yet another embodiment, the invention relates to compounds of the general formula X being substituted by one substituent R⁵.

In yet another embodiment, the invention relates to compounds of the general formula X being substituted by two independently selected R⁵ substituents.

In yet another embodiment, the invention relates to compounds of the general formula X being substituted by three independently selected R⁵ substituents.

Yet another aspect of the invention relates to compounds of the general formula XI or salts thereof: 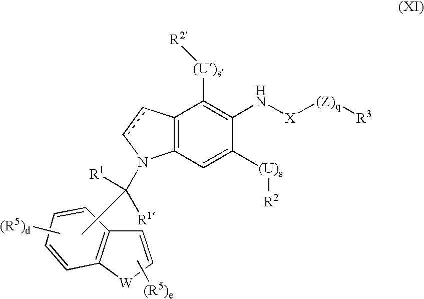
wherein the dotted line, d, e, q, s, U, X, Z, R¹, R^{1′}, R², R³ and R⁵ are as defined under formula I. Any of the embodiments related to formula I are also embodiments of formula XI.

In an embodiment, the invention relates to compounds of the general formula XI, wherein the nitrogen atom is attached to position 4 of the heteroaromatic group via the methylene group.

In another embodiment, the invention relates to compounds of the general formula XI, wherein the nitrogen atom is attached to position 5 of the heteroaromatic group via the methylene group.

In an embodiment, the invention relates to compounds of the general formula A, wherein the nitrogen atom is attached to position 6 of the heteroaromatic group via the methylene group.

In another embodiment, the invention relates to compounds of the general formula XI, wherein the nitrogen atom is attached to position 7 of the heteroaromatic group via the methylene group.

In yet another embodiment, the invention relates to compounds of the general formula X, wherein W is an oxygen atom.

In yet another embodiment, the invention relates to compounds of the general formula XI, wherein W is a sulphur atom.

In yet another embodiment, the invention relates to compounds of the general formula XI, wherein d is 0, 1 or 2, typically 0 or 1.

In yet another embodiment, the invention relates to compounds of the general formula XI, wherein e is 0, 1 or 2.

In yet another embodiment, the invention relates to compounds of the general formula A, wherein d+e is 0, 1, 2, 3 or 4.

In yet another embodiment, the invention relates to compounds of the general formula XI, wherein both d and e are 0.

In yet another embodiment, the invention relates to compounds of the general formula XI being substituted by one substituent R⁵.

In yet another embodiment, the invention relates to compounds of the general formula XI being substituted by two independently selected R⁵ substituents.

In yet another embodiment, the invention relates to compounds of the general formula XI being substituted by three independently selected R⁵ substituents.

Yet another aspect of the invention relates to compounds of the general formula XXXII or salts thereof: 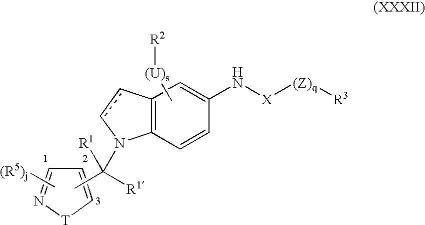
wherein the dotted line, j, q, s, T, U, X, Z, R¹, R^{1′}, R², R³ and R⁵ are as defined under formula I. Any of the embodiments related to formula I are also embodiments of formula XXXII.

In an embodiment, the invention relates to compounds of the general formula XXXII, wherein the nitrogen atom is attached to the position indicated with 1 of the heteroaromatic group via the methylene group.

In another embodiment, the invention relates to compounds of the general formula XXXII, wherein the nitrogen atom is attached to the position indicated with 2 of the heteroaromatic group via the methylene group.

In another embodiment, the invention relates to compounds of the general formula XXXII, wherein the nitrogen atom is attached to the position indicated with 3 of the heteroaromatic group via the methylene group.

In yet another embodiment, the invention relates to compounds of the general formula XXXII, wherein T is an oxygen atom.

In yet another embodiment, the invention relates to compounds of the general formula XXXII, wherein T is a nitrogen atom.

In yet another embodiment, the invention relates to compounds of the general formula XXXII, wherein T represents NH.

In another embodiment, the invention relates to compounds of the general formula XXXII, wherein j is 0, 1, 2 or 3.

In yet another embodiment, the invention relates to compounds of the general formula XXXII, wherein j is 0.

In yet another embodiment, the invention relates to compounds of the general formula XXXII being substituted by at least one substituent R⁵. In one aspect thereof, the compound of the general formula XXXII is substituted in the position indicated with 1. In another aspect thereof, the compound of the general formula XXXII is substituted in the position indicated with 2. In yet another aspect thereof, the compound of the general formula XXXII is substituted in the position indicated with 3. In yet another aspect thereof, T represents a nitrogen atom at which the compound of the general formula XXXII is substituted.

In yet another embodiment, the invention relates to compounds of the general formula XXXII being substituted by two independently selected R⁵ substituents.

In yet another embodiment, the invention relates to compounds of the general formula XXXII being substituted by two or three independently selected R⁵ substituents.

In yet another embodiment, the invention relates to compounds of the general formula XXXII being substituted by three independently selected R⁵ substituents.

One aspect of the invention, relates to compounds of general formula E m and salts thereof: 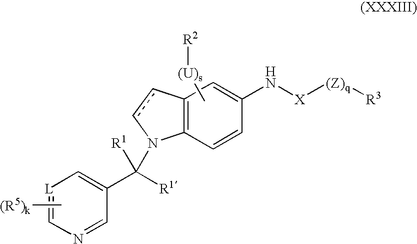
wherein the dotted line, k, q, s, L, U, Z, R¹, R^{1′}, R², R³ and R⁵ are as defined under formula I. Any of the embodiments related to formula I are also embodiments of formula XXXIII.

In one embodiment, the invention relates to compounds of the general formula XXXIII, wherein k is 0.

In another embodiment, the invention relates to compounds of the general formula XXXIII being substituted by one substituent R⁵, such as in the position being orto, meta or para to the nitrogen atom.

In a preferred embodiment, the invention relates to compounds of the general formula XXXIII, which are substituted by one substituent R⁵ in the position being para to the nitrogen atom.

In one embodiment, the invention relates to compounds of the general formula XXXIII being substituted by two independently selected R⁵ substituents, such as in the in the positions being ortho and para to the nitrogen atom, or in the positions being meta and para to the nitrogen atom, or in the positions being orto and meta to the nitrogen atom.

In another embodiment, the invention relates to compounds of the general formula XXXIII being substituted by three independently selected R⁵ substituents.

In one embodiment of the invention, the compounds of the following list and salts thereof are preferred:
N-[4-Chloro-1-(4-trifluoromethylbenzyl)-2,3-dihydro-1H-indol-5-yl]-3,3-dimethylbutyramide,
N-[4-Chloro-1-(5-chlorothiophen-2-ylmethyl)-2,3-dihydro-1H-indol-5-yl]-3,3-dimethylbutyramide,
[1-(4-Fluorobenzyl)-2,3-dihydro-1H-indol-5-yl]-carbamic acid propyl ester,
N-[1-(4-Fluorobenzyl)-2,3-dihydro-1H-indol-5-yl]-C-phenyl-methanesulfonamide,
4-Fluoro-N-[1-(4-fluorobenzyl)-2,3-dihydro-1H-indol-5-yl]-benzamide,
N-[1-(4-Fluorobenzyl)-2,3-dihydro-1H-indol-5-yl]-3,3-dimethylbutyramide,
N-[1-(4-Fluorobenzyl)-2,3-dihydro-1H-indol-5-yl]-2-thiophen-2-ylacetamide,
N-[1-(4-Fluorobenzyl)-2,3-dihydro-1H-indol-5-yl]-2-(4-fluorophenyl)-acetamide,
3-[1-(5-Chlorothiophen-2-ylmethyl)-2,3-dihydro-1H-indol-5-yl]-1,1-diisopropylurea,
Morpholine-4-carboxylic acid [1-(5-chlorothiophen-2-ylmethyl)-2,3-dihydro-1H-indol-5-yl]-amide,
Pyrrolidine-1-carboxylic acid [1-(5-chlorothiophen-2-ylmethyl)-2,3-dihydro-1H-indol-5-yl]-amide,
[1-(5-Chlorothiophen-2-ylmethyl)-2,3-dihydro-1H-indol-5-yl]-carbamic acid 2-benzyloxyethyl ester,
3-[1-(5-Chlorothiophen-2-ylmethyl)-2,3-dihydro-1H-indol-5-yl]-1-methyl-1-propylurea,
[1-(5-Chlorothiophen-2-ylmethyl)-2,3-dihydro-1H-indol-5-yl]-carbamic acid tert-butyl ester,
N-[1-(5-Chlorothiophen-2-ylmethyl)-2,3-dihydro-1H-indol-5-yl]-C-phenyl-methanesulfonamide,
Butane-1-sulfonic acid [1-(5-chlorothiophen-2-ylmethyl)-2,3-dihydro-1H-indol-5-yl]-amide,
N-[1-(5-Chlorothiophen-2-ylmethyl)-2,3-dihydro-1H-indol-5-yl]-4-fluorobenzamide,
N-[1-(5-Chlorothiophen-2-ylmethyl)-2,3-dihydro-1H-indol-5-yl]-2,2-dimethylpropionamide,
N-[1-(5-Chlorothiophen-2-ylmethyl)-2,3-dihydro-1H-indol-5-yl]-2-phenoxyacetamide,
N-[1-(5-Chlorothiophen-2-ylmethyl)-2,3-dihydro-1H-indol-5-yl]-3,3-dimethylbutyramide,
N-[1-(5-Chlorothiophen-2-ylmethyl)-2,3-dihydro-1H-indol-5-yl]-butyramide,
Cyclopentanecarboxylic acid [1-(5-chlorothiophen-2-ylmethyl)-2,3-dihydro-1H-indol-5-yl]-amide,
N-[1-(5-Chlorothiophen-2-ylmethyl)-2,3-dihydro-1H-indol-5-yl]-2-thiophen-2-ylacetamide,
N-[1-(5-Chlorothiophen-2-ylmethyl)-2,3-dihydro-1H-indol-5-yl]-isonicotinamide,
N-[1-(5-Chlorothiophen-2-ylmethyl)-2,3-dihydro-1H-indol-5-yl]-4-dimethylaminobenzamide,
N-[1-(5-Chlorothiophen-2-ylmethyl)-2,3-dihydro-1H-indol-5-yl]-2-(4-fluorophenyl)-acetamide,
N-[1-(5-Chlorothiophen-2-ylmethyl)-2,3-dihydro-1H-indol-5-yl]-6-trifluoromethylnicotinamide,
1-tert-Butyl-3-[1-(5-chlorothiophen-2-ylmethyl)-2,3-dihydro-1H-indol-5-yl]-urea,
1-[1-(5-Chlorothiophen-2-ylmethyl)-2,3-dihydro-1H-indol-5-yl]-3-ethylurea,
1-Benzyl-3-[1-(5-chlorothiophen-2-ylmethyl)-2,3-dihydro-1H-indol-5-yl]-urea,
1-[1-(5-Chlorothiophen-2-ylmethyl)-2,3-dihydro-1H-indol-5-yl]-3-phenethylurea,
1-[1-(5-Chlorothiophen-2-ylmethyl)-2,3-dihydro-1H-indol-5-yl]-3-thiophen-2-ylurea,
1-[1-(5-Chlorothiophen-2-ylmethyl)-2,3-dihydro-1H-indol-5-yl]-3-thiophen-3-ylurea,
2,2-Dimethyl-N-[6-nitro-1-(4-trifluoromethylbenzyl)-2,3-dihydro-1H-indol-5-yl]-propionamide,
N-[1-(5-Chlorothiophen-2-ylmethyl)-6-nitro-2,3-dihydro-1H-indol-5-yl]-2,2-dimethylpropionamide,
2-(4-Fluorophenyl)-N-[6-nitro-1-(4-trifluoromethylbenzyl)-2,3-dihydro-1H-indol-5-yl]-acetamide,
N-[6-Amino-1-(5-chlorothiophen-2-ylmethyl)-2,3-dihydro-1H-indol-5-yl]-3,3-dimethylbutyramide,
N-[6-Amino-1-(4-trifluoromethylbenzyl)-2,3-dihydro-1H-indol-5-yl]-2,2-dimethylpropionamide,
N-[6-Amino-1-(5-chlorothiophen-2-ylmethyl)-2,3-dihydro-1H-indol-5-yl]-2,2-dimethylpropionamide,
N-[6-Amino-1-(4-trifluoromethylbenzyl)-2,3-dihydro-1H-indol-5-yl]-2-(4-fluorophenyl)-acetamide, or
N-[1-(5-Chlorothiophen-2-ylmethyl)-1H-indol-5-yl]-3,3-dimethylbutyramide.

In another embodiment of the invention, the compounds of the following list and salts thereof are preferred:
N-[4-Chloro-1-(4-trifluoromethylbenzyl)-2,3-dihydro-1H-indol-5-yl]-3,3-dimethylbutyramide;
N-[4-Chloro-1-(5-chlorothiophen-2-ylmethyl)-2,3-dihydro-1H-indol-5-yl]-3,3-dimethylbutyramide;
[1-(4-Fluorobenzyl)-2,3-dihydro-1H-indol-5-yl]-carbamic acid propyl ester;
N-[1-(4-Fluorobenzyl)-2,3-dihydro-1H-indol-5-yl]-C-phenyl-methanesulfonamide;
4-Fluoro-N-[1-(4-fluorobenzyl)-2,3-dihydro-1H-indol-5-yl]-benzamide;
N-[1-(4-Fluorobenzyl)-2,3-dihydro-1H-indol-5-yl]-3,3-dimethylbutyramide;
N-[1-(4-Fluorobenzyl)-2,3-dihydro-1H-indol-5-yl]-2-thiophen-2-ylacetamide;
N-[1-(4-Fluorobenzyl)-2,3-dihydro-1H-indol-5-yl]-2-(4-fluorophenyl)-acetamide;
3-[1-(5-Chlorothiophen-2-ylmethyl)-2,3-dihydro-1H-indol-5-yl]-1,1-diisopropylurea;
Morpholine-4-carboxylic acid [1-(5-chlorothiophen-2-ylmethyl)-2,3-dihydro-1H-indol-5-yl]-amide;
Pyrrolidine-1-carboxylic acid [1-(5-chlorothiophen-2-ylmethyl)-2,3-dihydro-1H-indol-5-yl]-amide;
[1-(5-Chlorothiophen-2-ylmethyl)-2,3-dihydro-1H-indol-5-yl]-carbamic acid 2-benzyloxyethyl ester;
3-[1-(5-Chlorothiophen-2-ylmethyl)-2,3-dihydro-1H-indol-5-yl]-1-methyl-1-propylurea;
[1-(5-Chlorothiophen-2-ylmethyl)-2,3-dihydro-1H-indol-5-yl]-carbamic acid tert-butyl ester;
N-[1-(5-Chlorothiophen-2-ylmethyl)-2,3-dihydro-1H-indol-5-yl]-C-phenyl-methanesulfonamide;
Butane-1-sulfonic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-4-fluorobenzamide;
N-[1-(5-Chlorothiophen-2-ylmethyl)-2,3-dihydro-1H-indol-5-yl]-2,2-dimethylpropionamide;
N-[1-(5-Chlorothiophen-2-ylmethyl)-2,3-dihydro-1H-indol-5-yl]-2-phenoxyacetamide;
N-[1-(5-Chlorothiophen-2-ylmethyl)-2,3-dihydro-1H-indol-5-yl]-3,3-dimethylbutyramide;
N-[1-(5-Chlorothiophen-2-ylmethyl)-2,3-dihydro-1H-indol-5-yl]-butyramide;
Cyclopentanecarboxylic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-2-thiophen-2-ylacetamide;
N-[1-(5-Chlorothiophen-2-ylmethyl)-2,3-dihydro-1H-indol-5-yl]-isonicotinamide;
N-[1-(5-Chlorothiophen-2-ylmethyl)-2,3-dihydro-1H-indol-5-yl]-4-dimethylaminobenzamide;
N-[1-(5-Chlorothiophen-2-ylmethyl)-2,3-dihydro-1H-indol-5-yl]-2-(4-fluorophenyl)-acetamide;
N-[1-(5-Chlorothiophen-2-ylmethyl)-2,3-dihydro-1H-indol-5-yl]-6-trifluoromethylnicotinamide;
1-tert-Butyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-ethylurea;
1-Benzyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-phenethylurea;
1-[1-(5-Chlorothiophen-2-ylmethyl)-2,3-dihydro-1H-indol-5-yl]-3-thiophen-2-ylurea;
1-[1-(5-Chlorothiophen-2-ylmethyl)-2,3-dihydro-1H-indol-5-yl]-3-thiophen-3-ylurea;
[1-(5-Chlorothiophen-2-ylmethyl)-2,3-dihydro-1H-indol-5-yl]-carbamic acid propyl ester;
2,2-Dimethyl-N-[6-nitro-1-(4-trifluoromethylbenzyl)-2,3-dihydro-1H-indol-5-yl]-propionamide;
N-[1-(5-Chlorothiophen-2-ylmethyl)-6-nitro-2,3-dihydro-1H-indol-5-yl]-2,2-dimethylpropionamide;
2-(4-Fluorophenyl)-N-[6-nitro-1-(4-trifluoromethylbenzyl)-2,3-dihydro-1H-indol-5-yl]-acetamide;
N-[1-(5-Chlorothiophen-2-ylmethyl)-6-nitro-2,3-dihydro-1H-indol-5-yl]-2-(4 fluorophenyl)-acetamide;
N-[1-(5-Chlorothiophen-2-ylmethyl)-6-nitro-2,3-dihydro-1H-indol-5-yl]-3,3-dimethylbutyramide;
N-[6-Amino-1-(5-chlorothiophen-2-ylmethyl)-2,3-dihydro-1H-indol-5-yl]-3,3-dimethylbutyramide;
N-[6-Amino-1-(4-trifluoromethylbenzyl)-2,3-dihydro-1H-indol-5-yl]-2,2-dimethylpropionamide;
N-[6-Amino-1-(5-chlorothiophen-2-ylmethyl)-2,3-dihydro-1H-indol-5-yl]-2,2-dimethylpropionamide;
N-[6-Amino-1-(4-trifluoromethylbenzyl)-2,3-dihydro-1H-indol-5-yl]-2-(4-fluorophenyl)-acetamide;
N-[6-Amino-1-(4-trifluoromethylbenzyl)-2,3-dihydro-1H-indol-5-yl]-3,3-dimethylbutyramide;
N-[6-Amino-1-(4-fluorobenzyl)-2,3-dihydro-1H-indol-5-yl]-3,3-dimethylbutyramide;
N-[6-Amino-1-(3-fluoro-4-trifluoromethylbenzyl)-2,3-dihydro-1H-indol-5-yl]-3,3-dimethylbutyramide;
N-[1-(5-Chlorothiophen-2-ylmethyl)-1H-indol-5-yl]-3,3-dimethylbutyramide;
N-[6-Bromo-1-(4-trifluoromethylbenzyl)-2,3-dihydro-1H-indol-5-yl]-3,3-dimethylbutyramide;
N-[6-Bromo-1-(5-chlorothiophen-2-ylmethyl)-2,3-dihydro-1H-indol-5-yl]-3,3-dimethylbutyramide;
N-[1-(4-Chlorobenzyl)-2,3-dihydro-1H-indol-5-yl]-3,3-dimethylbutyramide;
3,3-Dimethyl-N-[1-(4-trifluoromethylbenzyl)-2,3-dihydro-1H-indol-5-yl]-butyramide;
N-[1-(4-Isopropylbenzyl)-2,3-dihydro-1H-indol-5-yl]-3,3-dimethylbutyramide;
N-[1-(3-Fluoro-4-trifluoromethylbenzyl)-2,3-dihydro-1H-indol-5-yl]-3,3-dimethylbutyramide;
N-[1-(6-Chlorobenzo[1,3]dioxol-5-ylmethyl)-2,3-dihydro-1H-indol-5-yl]-3,3-dimethylbutyramide;
N-[1-(3,5-Dimethyl-1-phenyl-1H-pyrazol-4-ylmethyl)-2,3-dihydro-1H-indol-5-yl]-3,3-dimethylbutyramide;
N-[1-(2-Chloro-5-trifluoromethylbenzyl)-2,3-dihydro-1H-indol-5-yl]-3,3-dimethylbutyramide;
N-{1-[5-(4-Chlorophenoxy)-1,3-dimethyl-1H-pyrazol-4-ylmethyl]-2,3-dihydro-1H-indol-5-yl}-3,3-dimethylbutyramide;
3,3-Dimethyl-N-[1-(6-p-tolyloxy-pyridin-3-ylmethyl)-2,3-dihydro-1H-indol-5-yl]-butyramide;
N-{1-[6-(4-Chlorophenylsulfanyl)-pyridin-3-ylmethyl]-2,3-dihydro-1H-indol-5-yl}-3,3-dimethylbutyramide;
N-{1-[6-(4-Cyanophenoxy)-pyridin-3-ylmethyl]-2,3-dihydro-1H-indol-5-yl}-3,3-dimethylbutyramide;
3,3-Dimethyl-N-[1-(6-trifluoromethylpyridin-3-ylmethyl)-2,3-dihydro-1H-indol-5-yl]-butyramide;
3,3-Dimethyl-N-[1-(3-methyl-benzo[b]thiophen-2-ylmethyl)-2,3-dihydro-1H-indol-5-yl]-butyramide;
N-[1-(6-Fluoro-4H-benzo[1,3]dioxin-8-ylmethyl)-2,3-dihydro-1H-indol-5-yl]-3,3-dimethylbutyramide;
3,3-Dimethyl-N-[1-(6-phenoxypyridin-3-ylmethyl)-2,3-dihydro-1H-indol-5-yl]-butyramide;
3,3-Dimethyl-N-[1-(3-methyl-5-phenyl-isoxazol-4-ylmethyl)-2,3-dihydro-1H-indol-5-yl]-butyramide;
N-(1-Benzo[b]thiophen-2-ylmethyl-2,3-dihydro-1H-indol-5-yl)-3,3-dimethylbutyramide;
N-{1-[1-(4-Fluorophenyl)-5-methyl-1H-pyrazol-4-ylmethyl]-2,3-dihydro-1H-indol-5-yl}-3,3-dimethylbutyramide;
3,3-Dimethyl-N-[1-(5-methylthiophen-2-ylmethyl)-2,3-dihydro-1H-indol-5-yl]-butyramide;
3,3-Dimethyl-N-[1-(4-pyrrol-1-yl-benzyl)-2,3-dihydro-1H-indol-5-yl]-butyramide;
N-[1-(4-Chlorobenzyl)-2,3-dihydro-1H-indol-5-yl]-2-(4-fluorophenyl)-acetamide;
2-(4-Fluorophenyl)-N-[1-(4-trifluoromethylbenzyl)-2,3-dihydro-1H-indol-5-yl]-acetamide;
2-(4-Fluorophenyl)-N-[1-(4-isopropylbenzyl)-2,3-dihydro-1H-indol-5-yl]-acetamide;
2-(4-Fluorophenyl)-N-[1-(3-fluoro-4-trifluoromethylbenzyl)-2,3-dihydro-1H-indol-5-yl]-acetamide;
N-[1-(6-Chlorobenzo[1,3]dioxol-5-ylmethyl)-2,3-dihydro-1H-indol-5-yl]-2-(4-fluorophenyl)-acetamide;
N-[1-(3,5-Dimethyl-1-phenyl-1H-pyrazol-4-ylmethyl)-2,3-dihydro-1H-indol-5-yl]-2-(4-fluorophenyl)-acetamide;
N-[1-(2-Chloro-5-trifluoromethylbenzyl)-2,3-dihydro-1H-indol-5-yl]-2-(4-fluorophenyl)-acetamide;
N-{1-[5-(4-Chlorophenoxy)-1,3-dimethyl-1H-pyrazol-4-ylmethyl]-2,3-dihydro-1H-indol-5-yl}-2-(4-fluorophenyl)-acetamide;
N-{1-[6-(4-Cyanophenoxy)-pyridin-3-ylmethyl]-2,3-dihydro-1H-indol-5-yl}-2-(4-fluorophenyl)-acetamide;
2-(4-Fluorophenyl)-N-[1-(3-methyl-benzo[b]thiophen-2-ylmethyl)-2,3-dihydro-1H-indol-5-yl]-acetamide;
N-[1-(6-Fluoro-4H-benzo[1,3]dioxin-8-ylmethyl)-2,3-dihydro-1H-indol-5-yl]-2-(4-fluorophenyl)-acetamide;
2-(4-Fluorophenyl)-N-[1-(6-phenoxypyridin-3-ylmethyl)-2,3-dihydro-1H-indol-5-yl]-acetamide;
N-(1-Benzo[b]thiophen-2-ylmethyl-2,3-dihydro-1H-indol-5-yl)-2-(4-fluorophenyl)-acetamide;
2-(4-Fluorophenyl)-N-{1-[1-(4-fluorophenyl)-5-methyl-1H-pyrazol-4-ylmethyl]-2,3-dihydro-1H-indol-5-yl}-acetamide;
2-(4-Fluorophenyl)-N-[1-(5-methylthiophen-2-ylmethyl)-2,3-dihydro-1H-indol-5-yl]-acetamide; and
2-(4-Fluorophenyl)-N-[1-(4-pyrrol-1-yl-benzyl)-2,3-dihydro-1H-indol-5-yl]-acetamide.

According to one embodiment, the invention relates to a pharmaceutical composition comprising one or more pharmaceutically acceptable carriers or diluents and a compound of formula I wherein the dotted line, s, q, U, X, Z, Y, R¹, R^{1′}, R² and R³ are as defined above, accordingly any of the dotted line, a, b, c, d, e, f, g, h, j, k, s, q, L, T, U X, Z, Y, W, R¹, R^{1′}, R², R³, R⁵, R⁶, R^{6′}, R⁷, R^{7′}, R⁸, R⁹, R^{9′}, R¹⁰, R¹¹, R^{11′}, R¹² and R^{12′} are as defined under formula I, or salts thereof. Pharmaceutical compositions of the invention may thus comprise one or more compounds of formula I or salts thereof, such as one compound of formula I or a salt thereof; or two compounds of formula I or salts thereof; or three compounds of formula I or salts thereof.

The invention thus provides a pharmaceutical composition for oral or parenteral administration, said pharmaceutical composition comprising at least one compound of formula I or a salt thereof in a therapeutically effective amount together with one or more pharmaceutically acceptable carriers or diluents.

In one aspect, the compounds of the invention may be administered as the only therapeutically effective compound.

In another aspect the compounds of the invention may be administered as a part of a combination therapy, i.e. the compounds of the invention may be administered in combination with other therapeutically effective compounds having e.g. anti-convulsive properties. The effects of such other compounds having anti-convulsive properties may include but not be limited to activities on:
ion channels such as sodium, potassium, or calcium channels
the excitatory amino acid systems e.g. blockade or modulation of NMDA receptors
the inhibitory neurotransmitter systems e.g. enhancement of GABA release, or blockade of GABA-uptake or
membrane stabilisation effects.

Current anti-convulsive medications include, but are not limited to, tiagabine, carbamazepine, sodium valproate, lamotrigine, gabapentin, pregabalin, ethosuximide, levetiracetam, phenyloin, topiramate, zonisamide as well as members of the benzodiazepine and barbiturate class.

In one aspect, the compounds of the invention have been found to have effect on potassium channels of the KCNQ family, in particular the KCNQ2 subunit.

In one embodiment, the invention relates to the use of one or more compounds according to the invention in a method of treatment. The disorder or condition to be prevented, treated or inhibited is responsive to an increased ion flow in a potassium channel such as the KCNQ family potassium ion channels. Such disorder or condition is preferably a disorder or condition of the central nervous system.

The compounds of the invention are considered useful for increasing ion flow in a voltage-dependent potassium channel in a mammal such as a human.

The compounds of the invention are considered useful for the prevention, treatment or inhibition of a disorder or condition being responsive to an increased ion flow in a potassium channel such as the KCNQ family potassium ion channels. Such disorder or condition is preferably a disorder or condition of the central nervous system.

The compounds of the invention are thus considered useful for preventing, treating or inhibiting disorders or diseases such as seizure disorders, neuropathic and migraine pain disorders, anxiety disorders and neurodegenerative disorders.

Accordingly, the compounds of the invention are considered useful for the prevention, treatment or inhibition of disorders or conditions such as convulsions, epilepsy, anxiety disorders, neuropathic pain and neurodegenerative disorders.

According to one particular embodiment, the compounds of the invention are thus considered to be useful for preventing, treating or inhibiting seizure disorders such as convulsions, epilepsy and status epilepticus.

In one embodiment, the compounds of the invention are considered useful in the prevention, treatment and inhibition of convulsions.

In another embodiment, the compounds of the invention are considered useful in the prevention, treatment and inhibition of epilepsy, epileptic syndromes and epileptic seizures.

In yet another embodiment, the compounds of the invention are considered useful in the prevention, treatment and inhibition of anxiety disorders such as anxiety and conditions and diseases related to panic attack, agoraphobia, panic disorder with agoraphobia, panic disorder without agoraphobia, agoraphobia without history of panic disorder, specific phobia, social phobia and other specific phobias, obsessive-compulsive disorder, posttraumatic stress disorder, acute stress disorders, generalized anxiety disorder, anxiety disorder due to general medical condition, substance-induced anxiety disorder, separation anxiety disorder, adjustment disorders, performance anxiety, hypochondriacal disorders, anxiety disorder due to general medical condition and substance-induced anxiety disorder and anxiety disorder not otherwise specified.

In yet another embodiment, the compounds of the invention are considered useful in the prevention, treatment and inhibition of anxiety disorders such as anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia, specific phobias, anxiety disorder due to general medical condition and substance-induced anxiety disorder.

In yet another embodiment, the compounds of the invention are also considered useful in the prevention, treatment and inhibition of neuropathic pain and migraine pain disorders such as allodynia, hyperalgesic pain, phantom pain, neuropathic pain related to diabetic neuropathy and neupathic pain related to migraine.

In yet another embodiment, the compounds of the invention are considered useful in the prevention, treatment and inhibition of neurodegenerative disorders such as Alzheimer's disease; Huntington's chorea; multiple sclerosis; amyotrophic lateral sclerosis; Creutzfeld-Jakob disease; Parkinson's disease; encephalopathies induced by AIDS or infection by rubella viruses, herpes viruses, borrelia and unknown pathogens; trauma-induced neurodegenerations; neuronal hyperexcitation states such as in medicament withdrawal or intoxication; and neurodegenerative diseases of the peripheral nervous system such as polyneuropathies and polyneuritides.

In yet another embodiment, the compounds of the invention are considered useful in the prevention, treatment and inhibition of neurodegenerative disorders such as Alzheimer's disease; Huntington's chorea; multiple sclerosis; amyotrophic lateral sclerosis; Creutzfeld-Jakob disease; Parkinson's disease; encephalopathies induced by AIDS or infection by rubella viruses, herpes viruses, borrelia and unknown pathogens; and trauma-induced neurodegenerations.

In yet another embodiment, the compounds of the invention are considered useful in the prevention, treatment and inhibition of neuronal hyperexcitation states such as in medicament withdrawal or intoxication.

The invention provides compounds showing effect in one or more of the following tests:
“Relative efflux through the KCNQ2 channel”
Which is a measure of the potency of the compound at the target channel
“Maximum electroshock”
Which is a measure of seizures induced by non-specific CNS stimulation by electrical means
“Pilocarpine induced seizures”
Seizures induced by pilocarpine are often difficult to treat with many existing antiseizure medications and so reflect a model of “drug resistant seizures”
“Electrical seizure-threshold tests” and “Chemical seizure-threshold tests”
These models measure the threshold at which seizures are initiated, thus being models that detect whether compounds could delay seizure initiation.
“Amygdala kindling”
Which is used as a measure of disease progression, as in normal animals the seizures in this model get more severe as the animal receives further stimulations.

According to one particular aspect of the invention, the compounds are KCNQ2 active with an EC₅₀ of less than 15000 nM such as less than 10000 nM as measured by the test “Relative efflux through the KCNQ2 channel” which is described below. According to another particular aspect of the invention, the compounds are KCNQ2 active with an EC₅₀ of less than 200 nM such as less than 1500 nM as measured by the test “Relative efflux through the KCNQ2 channel” which is described below.

According to yet another particular aspect of the invention, the compounds are KCNQ2 active with an EC₅₀ of less than 200 nM such as less than 150 nM as measured by the test “Relative efflux through the KCNQ2 channel” which is described below.

According to one particular aspect of the invention, the compounds have an ED₅₀ of less than 15 mg/kg in the test “Maximum electroshock” which is described below.

According to another particular aspect of the invention, the compounds have an ED₅₀ of less than 5 mg/kg in the test “Maximum electroshock” which is described below. According to one particular aspect of the invention, the compounds have an ED₅₀ of less than 5 mg/kg in the “Electrical seizure-threshold test” and “Chemical seizure-threshold test” which is described below.

Some compounds have few or clinically insignificant side effects. Some of the compounds are thus tested in models of the unwanted sedative, hypothermic and ataxic actions of the compounds.

Some of the compounds have a large therapeutic index between anticonvulsant efficacy and side-effects such as impairment of locomotor activity or ataxic effects as measured by performance on a rotating rod. This means that the compounds will expectedly be well tolerated in patients permitting high doses to be used before side effects are seen. Thereby compliance with the therapy will expectedly be good and administration of high doses may be permitted making the treatment more efficacious in patients who would otherwise have side effects with other medications.

Definitions

The term heteroatom refers to a nitrogen, oxygen or sulphur atom.

Halogen means fluoro, chloro, bromo or iodo.

The expressions C₁₋₆-alk(en/yn)yl and C₁₋₆-alk(an/en/yn)yl mean a C₁₋₆-alkyl, C₂₋₆-alkenyl or a C₂₋₆-alkynyl group. The term C₁₋₆-alkyl refers to a branched or un-branched alkyl group having from one to six carbon atoms inclusive, including but not limited to methyl, ethyl, 1-propyl, 2-propyl, 1-butyl, 2-butyl, 2-methyl-2-propyl and 2-methyl-1-propyl. Similarly, C₂₋₄-alkenyl and C₂₋₄-alkynyl, respectively, designate such groups having from two to six carbon atoms, including one double bond and one triple bond respectively, including but not limited to ethenyl, propenyl, butenyl, ethynyl, propynyl and butynyl.

The expression C₁₋₃-alk(en/yn)yl means a C₁₋₃-alkyl, C₂₋₃-alkenyl or a C₂₋₃-alkynyl group. The term C₁₋₃-alkyl refers to a branched or un-branched alkyl group having from one to three carbon atoms inclusive, including but not limited to methyl, ethyl, 1-propyl and 2-propyl. Similarly, C₂₋₃-alkenyl and C₂₋₃-alkynyl, respectively, designate such groups having from two to three carbon atoms, including one double bond and one triple bond respectively, including but not limited to ethenyl, 1-propenyl, 2-propenyl, 3-propenyl, ethynyl, 1-propynyl and 3-propynyl.

The expressions C₃₋₈-cycloalk(en)yl and C₃₋₈-cycloalk(an/en)yl mean a C₃₋₈-cycloalkyl- or cycloalkenyl group. The term C₃₋₈-cycloalkyl designates a monocyclic or bicyclic carbocycle having three to eight C-atoms, including but not limited to cyclopropyl, cyclobutyl, cyclopentyl, cyclohexyl, etc. The term C₃₋₈-cycloalkenyl designates a monocyclic or bicyclic carbocycle having three to eight C-atoms and including one double bond.

The expressions C₃₋₄-cycloalk(en)yl and C₃₋₆-cycloalk(an/en)yl mean a C₃₋₄-cycloalkyl- or cycloalkenyl group. The term C₃₋₆-cycloalkyl designates a monocyclic or bicyclic carbocycle having three to six C-atoms, including but not limited to cyclopropyl, cyclobutyl, cyclopentyl, cyclohexyl, etc.

The term heterocycloalk(en)yl designates a monocyclic or bicyclic ring system wherein the ring is formed by 4 to 8 atoms selected from 2-7 carbonatoms and 1 or 2 heteroatoms selected from N, S, or O.

When two substituents together with a carbon atom to which they are attached form a 3-8 membered saturated or unsaturated ring which optionally contains 1 or 2 heteroatoms, then a monocyclic ring system is formed by 3 to 8 atoms selected from 1-8 carbonatoms and 0-2 heteroatoms selected from N, S, or O. Examples of such ring systems are cyclopropyl, cyclobutyl, cyclopentyl, cyclohexyl.

The term halo-C₁₋₆-alk(en/yn)yl designates C₁₋₆-alk(en/yn)yl being substituted with one or more halogen atoms, including but not limited to trifluoromethyl. Similarly, halo-C₃₋₈-cycloalk(en)yl designates C₃₋₈-cycloalk(en)yl being substituted with one or more halogen atoms and halo-heterocycloalk(en)yl designates heterocycloalk(en)yl being substituted with one or more halogen atoms.

The term NR¹²R^{12′}—C₁₋₆-alk(en/yn)yl designates C₁₋₆-alk(en/yn)yl being substituted with NR¹²R^{12′}. The term NR¹²R^{12′}—C₃₋₈-cycloalk(en)yl designates C₃₋₈-cycloalk(en)yl being substituted with NR¹²R^{12′}. The term NR¹²R^{12′}—C₃₋₈-cycloalk(en)yl-C₁₋₆-alk(en/yn)yl designates C₃₋₈-cycloalk(en)yl-C₁₋₆-alk(en/yn)yl being substituted with NR¹²R^{12′}. When any of —NR¹²R^{12′}—C₁₋₆-alk(en/yn)yl, NR¹²R^{12′}—C₃₋₈-cycloalk(en)yl and NR¹²R^{12′}—C₃₋₈-cycloalk(en)yl-C₁₋₆-alk(en/yn)yl is optionally substituted, then any of C₁₋₆-alk(en/yn)yl, C₃₋₈-cycloalk(en)yl, C₃₋₈-cycloalk(en)yl-C₁₋₆-alk(en/yn)yl is optionally substituted with one or more substituents independently being C₁₋₆-alk(en/yn)yl, C₃₋₈-cycloalk(en)yl, C₃₋₈-cycloalk(en)yl-C₁₋₆-alk(en/yn)yl or Ar.

As used herein, the term acyl refers to formyl, C₁₋₆-alk(en/yn)ylcarbonyl, C₃₋₈-cycloalk(en)ylcarbonyl, Ar-carbonyl, Ar—C₁₋₆-alk(en/yn)ylcarbonyl or a C₃₋₈-cycloalk(en)yl-C₁₋₆-alk(en/yn)yl-carbonyl group, wherein C₁₋₆-alk(en/yn)yl, C₃₋₈-cycloalk(en)yl and Ar are as defined above.

When two substituents together with a nitrogen atom to which they are attached form a 4-8 membered saturated or unsaturated ring which optionally contains 1, 2 or 3 further heteroatoms, then a monocyclic ring system is formed by 4 to 8 atoms selected from the nitrogen atom, 1-7 carbonatoms and 0-3 further heteroatoms selected from N, S, or O. Examples of such ring systems are azetidine, beta-lactame, pyrrolidine, piperidine, piperazine, morpholine, pyrrole, oxazolidine, thiazolidine, imidazolidine, azetidine, beta-lactame, tetrazole and pyrazole.

When two adjacent substituents together with the aromatic group to which they are attached form a 4-8 membered ring, which optionally contains one or two heteroatoms, then a ring system is formed by 4-8 atoms selected from 3-8 carbonatoms and 0-2 heteroatoms selected from N, S, or O. Such two adjacent substituents may together form:

—(CH₂)_{n′}—CH₂—, —CH═CH—(CH₂)_{m″}—, —CH₂—CH═CH—(CH₂)_{p″}—, —CH═CH—CH═CH—, —(CH₂)_{n″}—O—, —O—(CH₂)_{m″}—O—, —CH₂—O—(CH₂)_{p″}—O—, —CH₂—O—CH₂—O—CH₂—, —(CH₂)_{n″}—S—, —S—(CH₂)_{m″}—S—, —CH₂—S—(CH₂)_{p″}—S—, —CH₂—S—CH₂—S—CH₂—, —(CH₂)_{n″}—NH—, —NH—(CH₂)_{m″}—NH—, —CH₂—NH—(CH₂)_{p″}—NH—, —CH═CH—NH—, —O—(CH₂)_{m″}—NH—, —CH₂—O—(CH₂)_{p″}—NH— or —O—(CH₂)_{p″}—NH—CH₂—, —S(CH₂)_{m″}—NH, —N═CH—NH—, —N═CH—O— or —N—CH—S—, wherein m″ is 1, 2 or 3, n″ is 2, 3 or 4 and p″ is 1 or 2.

The term Ar refers to optionally substituted aromatic systems of 5-10 carbon atoms, wherein 0, 1, 2, 3 or 4 carbon atoms may be replaced by heteroatoms independently selected from N, S, or O. Examples of such Ar groups are optionally substituted phenyl, optionally substituted naphtyl, optionally substituted pyridine, optionally substituted pyrrole, optionally substituted pyrimidine, optionally substituted quinoline, optionally substituted indole, optionally substituted thiophene, optionally substituted furan, optionally substituted thiazole and optionally substituted oxazole. Ar may be substituted with one or more substituents independently being hydroxy, halogen, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, C₁₋₆-alk(en/yn)yloxy, C₃₋₈-alk(en/yn)yloxy, acyl, nitro or cyano, —CO—NH—C₁₋₆-alk(en/yn)yl, —CO—N(C₁₋₆alk(en/yn)yl)₂, —NH₂, —NH—C₁₋₆-alk(en/yn)yl, —N(C₁₋₆-alk(en/yn)yl)₂, —S—C₁₋₆-alk(en/yn)yl, —SO₂—C₁₋₆-alk(en/yn)yl, —SO₂N(C₁₋₆-alk(en/yn)yl)₂ and —SO₂NH—C₁₋₆-alk(en/yn)yl; or two adjacent substituents may together with the aromatic group to which they are attached form a 48 membered ring, which optionally contains one or two heteroatoms and which may be saturated or unsaturated.

The terms C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy, C₂₋₆-alkenyloxy, C₂₋₆-alkynyloxy, C₃₋₈-cycloalk(en)yloxy, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆ alk(en/yn)ylcarbonyl, C₃₋₈-alk(en/yn)ylcarbonyl, Ar-carbonyl, Ar—C₁₋₆-alk(en/yn)ylcarbonyl, C₃₋₈-cycloalk(en)yl-C₁₋₆-alk(en/yn)ylcarbonyl, —CO—C₁₋₆-alk(en/yn)yl, —S—C₁₋₆-alk(en/yn)yl, —SO₂—C₁₋₆-alk(en/yn)yl and —SO₂O—C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, acyl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/Yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₄-alk(en/yn)yl-Ar, halo-C₁₋₆-alk(en/yn)yl-C₃₋₈-cycloalk(en)yl-Ar, halo-heterocycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl etc. designate such groups in which the C₁₋₆-alk(en/yn)yl, C₂₋₆-alkenyl, C₂₋₆-alkynyl, C₃₋₈-cycloalk(en)yl, heterocycloalk(en)yl, Ar, cyano, halo-C₁₋₆-alk(en/yn)yl, halo-C₃₋₈-cycloalk(en)yl, halo-heterocycloalk(en)yl and acyl are as defined above.

The salts of the invention are preferably pharmaceutically acceptable salts. Such salts include pharmaceutical acceptable acid addition salts, pharmaceutically acceptable metal salts, ammonium and alkylated ammonium salts.

The pharmaceutically acceptable salts of the invention are preferably acid addition salts. The acid addition salts of the invention are preferably pharmaceutically acceptable salts of the compounds of the invention formed with non-toxic acids. Acid addition salts include salts of inorganic acids as well as organic acids. Representative examples of suitable inorganic acids include hydrochloric, hydrobromic, hydroiodic, sulfuric, sulfamic, phosphoric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, ethanesulfonic, tartaric, ascorbic, pamoic, gluconic, citraconic, aspartic, stearic, palmitic, EDTA, glycolic, p-aminobenzoic, glutamic, bis-methylenesalicylic, methanesulfonic, ethanedisulfonic, itaconic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in J. Pharm. Sci. 1977, 66, 2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

Also intended as pharmaceutically acceptable acid addition salts are the hydrates, which the present compounds are able to form.

The compounds of the present invention may have one or more asymmetric centres and it is intended that any optical isomers, as separated, pure or partially purified optical isomers or racemic mixtures thereof are included within the scope of the invention.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

The compounds of this invention may exist in unsolvated as well as in solvated forms with solvents such as water, ethanol and the like. In general, the solvated forms are considered equivalent to the unsolvated forms for the purposes of this invention. Racemic forms can be resolved into the optical antipodes by known methods, for example, by separation of diastereomeric salts thereof with an optically active acid, and liberating the optically active amine compound by treatment with a base. Another method for resolving racemates into the optical antipodes is based upon chromatography on an optically active matrix. Racemic compounds of the present invention can also be resolved into their optical antipodes, e.g. by fractional crystallization of d- or l- (tartrates, mandelates or camphorsulphonate) salts. The compounds of the present invention may also be resolved by the formation of diastereomeric derivatives.

Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981).

Optically active compounds can also be prepared from optically active starting materials.

The invention also encompasses prodrugs of the present compounds, which on administration undergo chemical conversion by metabolic processes before becoming pharmacologically active substances. In general, such prodrugs will be functional derivatives of the compounds of the general formula I, VII, VIII, IX, X, XI, XXXII or XXXIII which are readily convertible in vivo into the required compound of the formula I, VII, VIII, IX, X, XI, XXXII or XXXIII. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

Whenever mentioned in relation to the compounds of the formulas I, VII, VIII, IX, X, XI, XXXI or XXXIII, the terms epilepsy and epilepsies embrace any of the epilepsies, epileptic syndromes and epileptic seizures referred to in International League Against Epilepsy: Proposal for revised clinical and electroencephalographic classification of epileptic seizures. Commission on Classification and Terminology of the International League Against Epilepsy. Epilepsia 1981 22: 489-501 and in International League Against Epilepsy: Proposal for revised classification of epilepsies and epileptic syndromes. Commission on Classification and Terminology of the International League Against Epilepsy. Epilepsia 1989 30(4): 389-399.

Whenever mentioned in relation to the compounds of the formulas I, VII, VIII, IX, X; XI, XXXII or XXXIII, the term anxiety disorders embraces conditions and diseases related to panic attack, agoraphobia, panic disorder with agoraphobia, panic disorder without agoraphobia, agoraphobia without history of panic disorder, specific phobia, social phobia, obsessive-compulsive disorder, posttraumatic stress disorder, acute stress disorders, generalized anxiety disorder, anxiety disorder due to general medical condition, substance-induced anxiety disorder, separation anxiety disorder, adjustment disorders and anxiety disorder not otherwise specified as defined by American Psychiatric Association Diagnostic and statistical manual of mental disorders, 4ed 1994: 110-113, 393-444 and 623-627.

Pharmaceutical Compositions

The compounds of this invention are generally utilized as the free base or as a pharmaceutically acceptable salt thereof. Representative examples are mentioned above.

If desired, the pharmaceutical composition of the invention may comprise the compound of formula I in combination with further pharmacologically active substances such as those described in the foregoing.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The pharmaceutical compositions of this invention or those which are manufactured in accordance with this invention may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients or other additives normally used in the art may be used.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is a base addition salt of a compound having the utility of a free acid. When a compound of the invention contains a free acid such salts may be prepared in a conventional manner by treating a solution or suspension of a free acid of the compound of the invention with a chemical equivalent of a pharmaceutically acceptable base. Representative examples are mentioned above.

For parenteral administration, solutions of the novel compounds of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions for injections may be prepared by dissolving the active ingredient and possible additives in a part of the solvent for injection, preferably sterile water, adjusting the solution to a desired volume, sterilising the solution and filling it in suitable ampules or vials. Any suitable additive conventionally used in the art may be added, such as tonicity agents, preservatives, antioxidants, etc.

Suitable pharmaceutical carriers include inert solid diluents or fillers, sterile aqueous solution and various organic solvents.

Examples of solid carriers are lactose, terra alba, sucrose, cyclodextrin, talc, agar, pectin, acacia, stearic acid and lower alkyl ethers of cellulose corn starch, potato starch, talcum, magnesium stearate, gelatine, lactose, gums, and the like.

Any other adjuvants or additives usually used for such purposes such as colourings, flavourings, preservatives etc. may be used provided that they are compatible with the active ingredients.

Examples of liquid carriers are syrup, peanut oil, olive oil, phospho lipids, fatty acids, fatty acid amines, polyoxyethylene and water. Similarly, the carrier or diluent may include any sustained release material known in the art, such as glyceryl monostearate or glyceryl distearate, alone or mixed with a wax.

The pharmaceutical compositions formed by combining the novel compounds of the is invention and the pharmaceutical acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include one or more suitable excipients. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablette, placed in a hard gelatine capsule in powder or pellet form or it can be in the form of a troche or lozenge.

The amount of solid carrier will vary widely but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

If desired, the pharmaceutical composition of the invention may comprise the compound of the formula I, VII, VIII, IX, X or XI in combination with further pharmacologically active substances such as those described in the foregoing.

Typical examples of recipes for the formulation of the invention are as follows:

| 1) Tablets containing 5.0 mg of a compound of | | | |
|---|---|---|---|
| the invention calculated as the free base: | | | |
| Compound of formula I, VII, VIII, IX, X or XI | | | 5.0 mg |
| Lactose | 60 | mg | |
| Maize starch | 30 | mg | |
| Hydroxypropylcellulose | 2.4 | mg | |
| Microcrystalline cellulose | 19.2 | mg | |
| Croscarmellose Sodium Type A | 2.4 | mg | |
| Magnesium stearate | 0.84 | mg | |
| 2) Tablets containing 0.5 mg of a compound of | | | |
| the invention calculated as the free base: | | | |
| Compound of formula I, I, VII, VIII, IX, X or XI | | | 0.5 mg |
| Lactose | 46.9 | mg | |
| Maize starch | 23.5 | mg | |
| Povidone | 1.8 | mg | |
| Microcrystalline cellulose | 14.4 | mg | |
| Croscarmellose Sodium Type A | 1.8 | mg | |
| Magnesium stearate | 0.63 | mg | |
| 3) Syrup containing per millilitre: | | | |
| Compound of formula I, VII, VIII, IX, X or XI | | | 25 mg |
| Sorbitol | 500 | mg | |
| Hydroxypropylcellulose | 15 | mg | |
| Glycerol | 50 | mg | |
| Methyl-paraben | 1 | mg | |
| Propyl-paraben | 0.1 | mg | |
| Ethanol | 0.005 | mL | |
| Flavour | 0.05 | mg | |
| Saccharin sodium | 0.5 | mg | |
| Water | ad 1 | mL | |
| 4) Solution for injection containing per millilitre: | | | |
| Compound of formula I, VII, VIII, IX, X or XI | | | 0.5 mg |
| Sorbitol | 5.1 | mg | |
| Acetic Acid | 0.05 | mg | |
| Saccharin sodium | 0.5 | mg | |
| Water | | | |

Preparation of the Compounds of the Invention

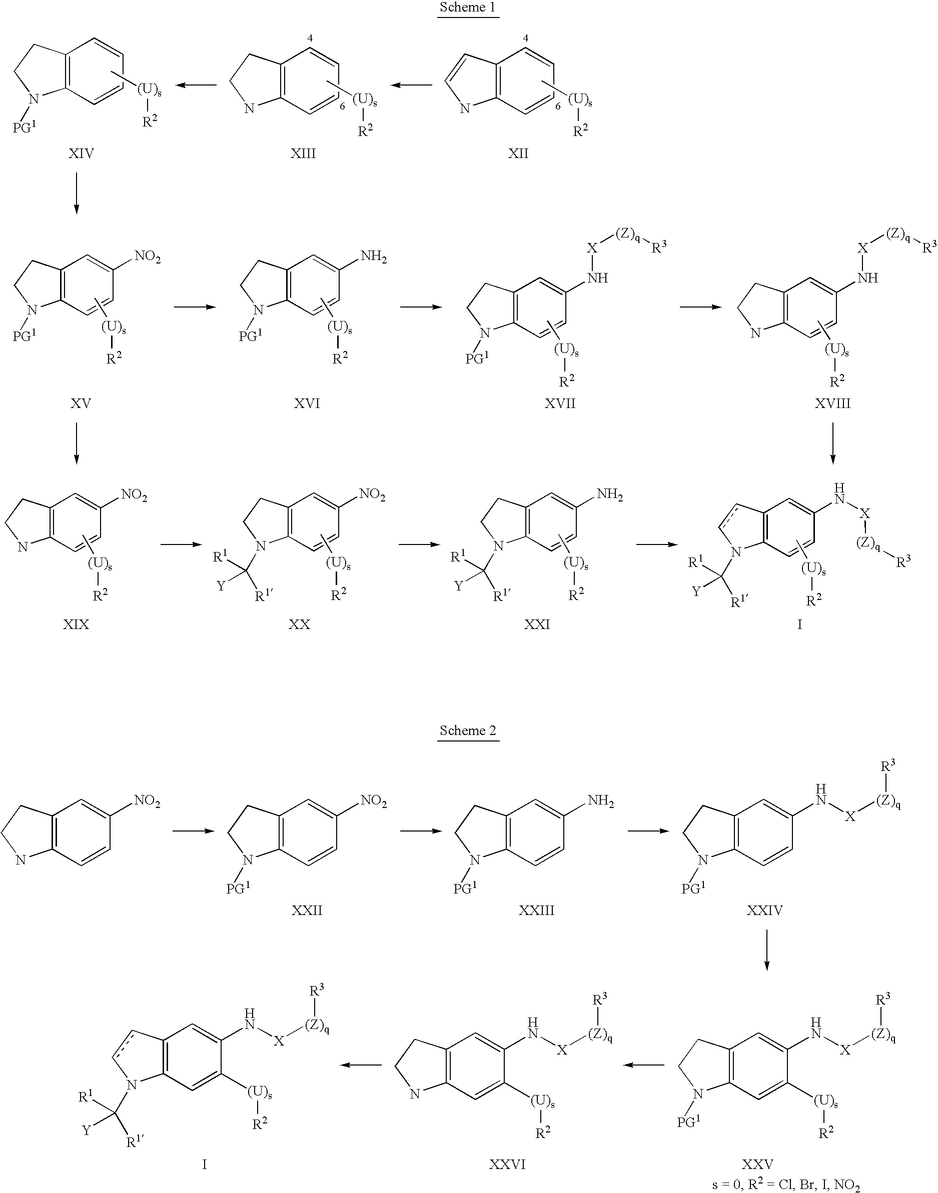

The compounds of the invention of the general formula I, wherein the the dotted line, q, s, U, Y, X, Z, R¹, R^{1′}, R² and R³ are as defined above, accordingly any of dotted line, a, b, c, d, e, f, g, h, s, q, U, X, Z, Y, W, R¹, R^{1′}, R², R³, R⁵, R⁶, R^{6′}, R⁷, R^{7′}, R⁸, R⁹, R^{9′}, R¹⁰, R^{10′}, R¹¹, R¹² and R^{12′} are defined under formula I are prepared by the methods as described below and as represented in the Schemes 1 and 2.

Indoles and indolines of the general formula XII and XIII substituted at position 4 or 6 with R²—(U)ₛ— are commercially available, described in the literature or prepared according to methods known to chemists skilled in the art [R. J. Sundberg “Pyrroles and their Benzo Derivatives: (iii) Synthesis and Applications” in Comprehensive Heterocyclic Chemistry, A. R. Katritzky, C. W. Rees (Editors), vol. IV, pp 313-376, Pergamon Press, 1984]. Indoles of the general formula XII can be converted into indolines of the general formula XIII by methods known to chemists skilled in the art such as catalytic hydrogenation or reduction with NaBH₃CN in appropriate solvents such as acetic acid [S. M. Bromidge, S. Dabbs, D. T. Davies, D. M. Duckworth, I. T. Forbes et al. J. Med. Chem. 41, 1998, 1598-1612]. Compounds of the general formula XII or XIII with s being 0 and R² being in particular but not limited to substituted aryl or substituted heteroaryl as defined above can be prepared from corresponding compounds with R² being I or Br by means of C—C coupling reactions known to chemists skilled in the art, such as Suzuki coupling, Stille coupling, or other transition metal catalysed cross-coupling reactions [D. W. Knight “Coupling Reactions Between sp2 Carbon Centers” in Comprehensive Organic Synthesis, v. 3, pp. 481-520, Pergamon Press 1991].

Compounds of the general formula XIV are prepared by protection of the indoline nitrogen of the compounds of the general formula XIII with an appropriate protecting group (PG¹) [Protective Groups in Organic Synthesis, 3rd Edition T. W. Greene, P. G. M. Wuts, Wiley Interscience 1999], such as a trifluoroacetyl group known to chemists skilled in the art as TFA group, by reaction with the reagent forming the protective group such as trifluoroacetic acid anhydride in a suitable solvent, such as 1,2-dichloroethane at appropriate temperatures.

The obtained compounds of the general formula XIV are converted into compounds of the general formula XV by regioselective nitration at position 5 of the indoline moiety by methods known to chemists skilled in the art [R. Behnisch “Aromatische Nitro-Verbindungen” in Methoden der Organische Chemie/(Houben-Weyl) p. 255, v. E16d, Thieme: 1992] such as reaction with concentrated nitric acid in appropriate solvent such as acetic anhydride, acetic acid, concentrated sulphuric acid or mixtures thereof at appropriate temperatures. The nitro compounds of the general formula XV where R² is halogen, in particular fluorine, and s is 0 can be converted into compounds of the general formula XV, where U is O, NR¹¹ or S and R² is as defined above, by nucleophilic aromatic substitution reactions known to chemists skilled in the art such as reaction with the appropriate nucleophiles forming the —(U)ₛ—R² group such as thiophenols, alkylsulfides, alcohols, phenols, amines, and anilines in their neutral or deprotonated form. The compounds of the general formula XV where U is SO₂ can be obtained from the compounds of the general formula XV, where U is S, by oxidation according to methods known to the chemist skilled in the art, for example by oxidation with NaIO₄ in the presence of RuCl₃ as a catalyst or with 3-chloroperoxybenzoic acid.

The nitro group in compounds of the general formula XV can be reduced with suitable reducing agents such as zinc or iron powder in the presence of acid such as acetic acid or aqueous hydrochloric acid, or hydrogen gas or ammonium formiate in the presence of a suitable hydrogenation catalyst such as palladium on activated carbon in suitable solvents such as methanol, ethanol, or tetrahydrofuran, at suitable temperatures or under ultrasonic irradiation, to obtain anilines of the general formula XVI. Alternatively, tin (II) chloride or sodium dithionite can be used as reducing agents under conditions well known to the chemist skilled in the art.

Compounds of the general formula XVII are prepared from compounds of the general formula XVI by the reaction with suitable electrophilic reagents forming an R³-(Z)_{q}-X group, such as alkyl, aryl or heteroaryl chloroformiates or carbamyl chlorides, acid chlorides, acid bromides, acid iodides, sulfonyl chlorides, isocyanates, carbonic acid anhydrides, activated carbonic acids with activating reagents such as carbodiimides or others as known to chemists skilled in the art in suitable solvents, such as acetonitrile, tetrahydrofuran, 1,2-dichloroethane, or methylene chloride, at suitable temperature, such as room temperature or reflux, with or without addition of bases, such as magnesium oxide, potassium carbonate, sodium hydride, trialkylamines, sodium- or potassium alcoholates, or pyridine, reactions well known to the chemist skilled in the art. Then the protective group PG¹ is removed according to methods known to chemists skilled in the art [Protective Groups in Organic Synthesis, 3rd Edition T. W. Greene, P. G. M. Wuts, Wiley Interscience 1999], furnishing compounds of the general formula XVIII. For example, when PG¹ is TFA, it can be removed by hydrolysis with aqueous potassium carbonate in an appropriate solvent, such as methanol, at a suitable temperature.

Finally, the obtained anilines of the general formula XVIII are subjected to reductive alkylation reactions, known to chemists skilled in the art, with aldehydes of the general formula YCHO where Y is defined as above in the presence of suitable reducing agent such as NaBH₃CN in suitable solvents such as methanol, ethanol, tetrahydrofuran, acetonitrile or mixtures thereof, with or without addition of catalytic amounts of acid, such as acetic acid, at suitable temperatures forming compounds of the invention of the general formula I, where R¹ and R^{1′} are hydrogens. Alternatively, a (Y)(R¹)(R^{1′})C— group can be introduced by nucleophilic substitution reactions with the appropriate electrophiles of the general formula (Y)(R¹)(R¹′)C-LG, where LG is a suitable leaving group such as iodide, bromide, or sulphonate, under conditions known to the chemist skilled in the art, finishing the compounds of the invention of the general formula I.

Alternatively, compounds of the general formula XIX are commercially available, described in the literature or can be prepared from compounds of the general formula XV by deprotection as described above. Then they are subjected to reductive alkylation with aldehydes of the general formula YCHO or to nucleophilic substitution reactions with electrophiles of the general formula (Y)(R¹)(R¹′)C-LG as described above, furnishing compounds of the general formula XX. Then the nitro group is reduced as described above forming compounds of the general formula XXI. Finally, the compounds of the invention of the general formula I with indoline moiety are obtained by the method described above for the conversion of compounds of the general formula XVI into compounds of the general formula XVII.

Optionally, compounds of the invention of the general formula I with indole moiety can be obtained from indolines of the general formula I by means of dehydrogenation known to chemists skilled in the art such as oxidation with appropriate reagents such as 2,3,5,6-tetrachloro-[1,4]benzoquinone, MnO₂, or catalytic dehydrogenation in the presence of a catalyst such as Pd on charcoal or RuCl₂(PPh₃)₃ in appropriate solvents such as toluene or xylene at appropriate temperatures.

Alternatively, compounds of the general formula I where —(U)ₛ—R² is attached to the position 6 of the indoline moiety, can be prepared by a route shown in Scheme 2 as follows:

5-Nitroindoline is protected with an appropriate protecting group, such as TFA group, as described above for compounds of the general formula XIV, furnishing compounds of the general formula XXII. Then the nitro group is reduced as described above for preparation of compounds of the general formula XVI, furnishing compounds of the general formula XXII. They are converted into compounds of the general formula XXIV With appropriate electrophiles forming R³-(Z)_{q}-X as described above for compounds of the general formula XVII. Compounds of the general formula XXV where s is 0 and R² is NO₂ or halogen such as Cl, Br or I, are obtained by means of regioselective electrophilic aromatic substitution, well known to chemists skilled in the art, with appropriate electrophiles such as N-chlorosuccinimide, bromine, iodine, iodochloride in the appropriate solvent such as acetic acid or by nitration under conditions as described for compounds of the general formula XV.

Compounds of the general formula XXV where s is 0 and R² is substituted aryl or substituted heteroaryl as defined above can be prepared from corresponding compounds of the same general formula where R² is I or Br by means of C—C coupling reactions known to chemists skilled in the art as described above. Then the protective group is removed as described above, furnishing the compounds of the general formula XXVI.

Finally, the compounds of the invention of the general formula I with indoline moiety are prepared from the compounds of the general formula XXVI by reductive alkylation or by nucleophilic substitution reactions as described above. Also, the compounds of the invention of the general formula I with indole moiety can be obtained from indolines of the general formula I by means of dehydrogenation as described above.

### EXAMPLES

Analytical LC-MS data were obtained on a PE Sciex API 150EX instrument equipped with an APPI (atmospheric pressure photo ionisation) ion source and Shimadzu LC-8A/SLC-10A LC system. Column: 30×4.6 mm Waters Symmetry C18 column with 3.5 μm particle size; Solvent system: A=water/trifluoroacetic acid (100:0.05) and B=water/acetonitrile/trifluoroacetic acid (5:95:0.03); Method: Linear gradient elution with 90% A to 100% B in 4 minutes and with a flow rate of 2 mL/minute. Purity was determined by integration of the UV (254 nm) and ELSD trace. The retention times (RT) are expressed in minutes.

Preparative LC-MS-purification was performed on the same instrument. Column: 50×20 mm YMC ODS-A with 5 μm particle size; Method: Linear gradient elution with 80% A to 100% B in 7 minutes and with a flow rate of 22.7 mL/minute. Fraction collection was performed by split-flow MS detection.

¹H NMR spectra were recorded at 500.13 MHz on a Bruker Avance DRX500 instrument. Deuterated chloroform (99.8% D) or dimethyl sulfoxide (99.8% D) were used as solvents. TMS was used as internal reference standard. Chemical shift values are expressed in ppm-values. The following abbreviations are used for multiplicity of NMR signals: s=singlet, d=doublet, t=triplet, q=quartet, qui=quintet, h=heptet, dd=double doublet, dt=double triplet, dq=double quartet, ft=triplet of triplets, m=multiplet and br.=broad.

Preparation of Intermediates

Preparation of Intermediates of the General Formula XXI and XIV

### 1-Trifluoroacetyl-5-nitroindoline

To a suspension of 5-nitroindoline (5.51 g, 33.56 mmol) in 1,2-dichloroethane (15 ml) trifluoroacetic anhydride (20 ml) was added. After 60 min the obtained solution was quenched with heptane (200 ml) and the title compound was separated by filtration in two crops. Yield 7.12 g, 81.5%. ¹H NMR (DMSO-d₆): 3.34 (t, 2H), 4.38 (t, 2H), 8.19 (m, 3H).

### 1-Trifluoroacetyl-4-chloroindoline was prepared analogously from 4-chloroindoline

[S. M. Bromidge, S. Dabbs, D. T. Davies, D. M. Duckworth, I. T. Forbes et al. J. Med. Chem. 41, 1998, 1598-1612]. ¹H NMR (DMSO-d₆): 3.26 (t, 2H), 4.34 (t, 2H), 7.27 (d, 1H), 7.34 (t, 1H), 8.01 (d, 1H).

Preparation of Intermediates of the General Formula XV

### 1-Trifluoroacetyl-4-chloro-5-nitroindoline

To a solution of 1-trifluoroacetyl-4-chloroindoline (197 mg, 0.838 mmol) in acetic anhydride (3 ml) and acetic acid (0.3 ml) a solution of fuming HNO₃ (0.4 ml) was added by small portions during 5 hours. The resulting reaction mixture was poured into ice, neutralised with saturated aqueous NaHCO₃, and extracted with ethyl acetate. The organic solution was filtered via plug of SiO₂ (10 g), evaporated in vacuo and purified by flash chromatography on SiO₂ with gradient heptane-1:4 ethyl acetate/heptane to give 70 mg of the title compound as yellow solid, yield 31%. ¹H NMR (DMSO-d₆): 3.33 (t, 2H), 4.42 (t, 2H), 8.10 (s, 2H).

Preparation of Intermediates of the General Formula XX

### 1-(5-Chlorothiophen-2-ylmethyl)-5-nitroindoline

To a solution of 5-nitroindoline (3.23 g, 19.67 mmol) and 5-chlorothiophene-2-carboxaldehyde (4.2 g, 28.6 mmol) in methanol (45 ml) and acetic acid (8 ml) a solution of NaBH₃CN (0.9 g) in methanol (8 ml) was added dropwise during 10 min. he obtained reaction mixture was stirred overnight. The title compound was separated by filtration, washed with methanol and water and dried in vacuo to furnish 4.6 g of red crystalline solid. Yield 79.3%. LC/MS (m/z) 293.9 ([M]⁺); RT=3.59, (UV, ELSD) 98%, 99.8%. ¹H NMR (DMSO-d₆): 3.04 (t, 2H), 3.62 (t, 2H), 4.68 (s, 2H), 6.72 (d, 1H), 6.98 (d, 1M), 7.01 (d, 1H), 7.85 (unresolved m, 1H), 8.00 (dd, 1H).

The following compound was prepared analogously using appropriate aldehydes:

### 1-(4-Fluorobenzyl)-5-nitroindoline

Yellow needles, yield 3.66 g, 72.2%. LC/MS (m/z) 272.0 ([M]⁺); RT=3.35, (UV, ELSD) 99%, 100%. ¹H NMR (DMSO-d₆): 3.06 (t, 2H), 3.61 (t, 2H), 4.52 (s, 2H), 6.63 (d, 1H), 7.18 (m, 2H), 7.35 (m, 2H), 7.83 (unresolved m, 1H), 7.97 (dd, 1H).

Preparation of Intermediates of the General Formula XXI, XXI, and XVI

### 1-(5-Chlorothiophen-2-ylmethyl)-5-aminoindoline

To a cold (ice/water bath) vigorously stirred solution of 1-(5-Chlorothiophen-2-ylmethyl)-5-nitroindoline (4.013 g, 13.62 mmol) in THF (100 ml) and acetic acid (15 ml) zinc powder (25 g) was added by small portions maintaining the temperature below 40° C. The cold bath removed and the stirring continued at room temperate until reaction completion (1 hour). The obtained suspension was filtered via a plug of SiO₂ (25 g) with ethyl acetate as an eluent and obtained solution was evaporated in vacuo. The obtained residue was treated with saturated aqueous NaHCO₃, extracted with ethyl acetate, dried over Na₂SO₄ and evaporated in vacuo to give the title compound as a dark green oil. Yield 3.30 g, 91.5%. LC/MS (m/z) 265.9 ([M+1]⁺); RT=1.85, (UV, ELSD) 93%, 100%. ¹H NMR (DMSO-d₆): 2.73 (t, 2H), 3.08 (t, 2H), 4.25 (s, 2H), 4.40 (br. s, 2H, NH₂), 6.30 (dd, 1H), 6.41 (d, 1H), 6.43 (unresolved m, 1H), 6.89 (d, 1H), 6.95 (d, 1H).

The following compounds were prepared analogously:

### 1-(4-Fluorobenzyl)-5-aminoindoline

The obtained crude product after filtation via SiO₂ was dissolved in a small amount of methanol, quenched with saturated aqueous NaHCO₃, and the title compound was separated by filtration, washed with water, and dried in vacuo. Yield 2.40 g, 93.2%, dark violet solid. LC/MS (m/z) 265.9 ([M+1]⁺); RT=1.74, (UV, ELSD) 87%, 98%. ¹H NMR (DMSO-d₆): 2.72 (t, 2H), 3.01 (t, 2H), 4.04 (s, 2H), 4.36 (br. s, 2H, NH₂), 6.28 (d, 1H), 6.34 (d, 1H), 6.44 (s, 1H), 7.14 (t, 2H), 7.38 (t, 2H).

### 1-Trifluoroacetyl-5-aminoindoline

The title compound was prepared from 1-trifluoroacetyl-5-nitroindoline (6.67 g, 25.65 mmol). The crude product after filtration via SiO₂ was used in the next step without purification. Yield 6.11 g, 100%. LC/MS (m/z) 230.1 ([M]⁺); RT=1.29, (UV, ELSD) 97%, 98%. ¹H NMR (DMSO-d₆): 3.10 (t, 2H), 4.18 (t, 2H), 5.18 (br. s, 2H, NH₂), 6.43 (dd, 1H), 6.53 (s, 1H), 7.75 (d, 1H).

### 1-Trifluoroacetyl-4-chloro-5-aminoindoline

¹H NMR (CDCl₃): 3.23 (t, 2H), 4.28 (t, 2H), 6.67 (d, 1H), 7.93 (d, 1H).

Preparation of Intermediates of the General Formula XXV and XVII

### 3,3-Dimethyl-N-[1-(2,2,2-trifluoroacetyl)-2,3-dihydro-1H-indol-5-yl]-butyramide

To a cold (ice/water bath) solution of 1-trifluoroacetyl-5-aminoindoline (2.69 g, 11.7 mmol) in CH₂Cl₂ tert-butylacetyl chloride (1.88 g, 14 mmol) was added followed by addition of Et₃N (4 ml). After 5 min the reaction mixture was quenched with saturated aqueous NaHCO₃ and stirred for 30 min. The organic layer was filtered via plug of SiO₂ (20 g) with ethyl acetate as an eluent and evaporated to a small volume. It was quenched with heptane and the title compound was separated by filtration. Yield 3.10 g, 81%, white solid. LC/MS (m/z) 329.2 ([M+1]⁺); RT=3.04, (UV, ELSD) 97%, 100%. ¹H NMR (DMSO-d₆): 1.02 (s, 9H), 2.18 (s, 2H), 3.22 (t, 2H), 4.26 (t, 2H), 7.38 (dd, 1H), 7.72 (s, 1H), 7.96 (d, 1H), 9.86 (s, 1H, NHCO).

The following compounds were prepared analogously from 1-trifluoroacetyl-5-aminoindoline and appropriate acid chloride or chloroformiate:

### N-[4-Chloro-1-(2,2,2-trifluoroacetyl)-2,3-dihydro-1H-indol-5-yl]-3,3-dimethylbutyramide was prepared from 1-trifluoroacetyl-4-chloro-5-aminoindoline

The reaction mixture was evaporated and used in the next step without characterisation.

### 2,2-Dimethyl-N-[1-(2,2,2-trifluoroacetyl)-2,3-dihydro-1H-indol-5-yl]-propionamide

¹H NMR (DMSO-d₆): 1.22 (s, 9H), 3.23 (t, 2H), 4.28 (t, 2H), 7.47 (dd, 1H), 7.71 (s, 1H), 7.96 (d, 1H), 9.26 (s, 1H, NHCO).

### 2-(4-Fluorophenyl)-N-[1-(2,2,2-trifluoroacetyl)-2,3-dihydro-1H-indol-5-yl]-acetamide

LC/MS (m/z) 367.0 ([M+1]⁺); RT=3.00, (UV, ELSD) 92%, 99%. ¹H NMR (DMSO-d₆): 3.22 (t, 2H), 3.63 (s, 2H), 4.27 (t, 21, 7.15 (t, 2H), 7.36 (dd, 2H), 7.39 (dd, 1H), 7.69 (s, 1H), 7.97 (d, 1H), 10.24 (s, 1H, NHCO).

### [1-(2,2,2-Trifluoroacetyl)-2,3-dihydro-1H-indol-5-yl]-carbamic acid ethyl ester

The title compound was prepared using 1,2-dichloroethane as a solvent and pyridine as a base. LC/MS (m/z) 302.1 ([M]⁺); RT=2.85 (UV, ELSD) 79%, 100%. ¹H NMR (DMSO-d₆): 1.24 (t, 3H), 3.22 (t, 2H), 4.12 (q, 2H), 4.26 (t, 2H), 7.31 (br. d (unresolved dd), 1H), 7.49 (s, 1H), 7.94 (d, 1H), 9.70 (s, 1H, NHCO).

### [1-(2,2,2-Trifluoroacetyl)-2,3-dihydro-1H-indol-5-yl]-carbamic acid propyl ester

The title compound was prepared using 1,2-dichloroethane as a solvent and pyridine as a base. LC/MS (m/z) 315.9 ([M]⁺); RT=3.11 (UV, ELSD) 89%, 99%. ¹H NMR (DMSO-d₆): 0.93 (t, 3H), 1.64 (m, 2H), 3.22 (t, 2H), 4.03 (t, 2H), 4.26 (t, 2H), 7.32 (br. d (unresolved dd), 1H), 7.50 (s, 1H), 7.94 (d, 1H), 9.71 (s, 1H, NHCO).

Preparation of Intermediates of the General Formula XXV and XXVI

### 3,3-Dimethyl-N-(6-nitro-2,3-dihydro-1H-indol-5-yl)-butyramide

To a cold (ice/water bath) stirred solution of 3,3-dimethyl-N-[1-(2,2,2-trifluoroacetyl)-2,3-dihydro-1H-indol-5-yl]-butyramide (1.96 g, 5.98 mmol) in acetic anhydride (30 ml) and acetic acid (5 ml) a solution of fuming HNO₃ (650 mg, 10.3 mmol) in acetic acid (5 ml) was added dropwise during 5 min. After 5 min the reaction mixture was poured into ice and neutralised with solid NaHCO₃ which was added by small portions with stirring until gas formation ceased. The yellow solid of 3,3-dimethyl-N-[6-nitro-1-(2,2,2-trifluoroacetyl)-2,3-dihydro-1H-indol-5-yl]-butyramide was filtered, washed with water and dried in vacuo. LC/MS (m/z) 374.0 ([M+1]⁺); RT=3.45 (UV, ELSD) 94%, 99%. ¹H NMR (DMSO-d₆): 1.03 (s, 9H), 2.23 (s, 2H), 3.35 (t, 2H), 4.36 (t, 2H), 7.66 (s, 1H), 7.51 (s, 1H), 10.17 (s, 1H, NHCO).

The solid was redissolved in methanol (30 ml) followed by addition of K₂CO₃ (2.0 g) in water (7 ml). The colour changed immediately from yellow to dark red. After stirring for 15 min the reaction mixture was poured into ice/water and the title compound was isolated by filtration to give 1.52 g of purple solid, yield 91.8%. LC/MS (m/z) 277.0 ([M]⁺); RT=2.30 (UV, ELSD) 91%, 99%. ¹H NMR (CDCl₃): 1.10 (s, 9H), 2.29 (s, 2H), 3.10 (t, 2H), 3.63 (t, 2H), 4.80 (very br. s, NH), 7.30 (s, 1H), 8.46 (s, 1H), 10.14 (s, 1H, NHCO).

The following compounds were prepared analogously:

### 2,2-Dimethyl-N-(6-nitro-2,3-dihydro-1H-indol-5-yl)-propionamide

LC/MS (m/z) 264.1 ([M+1]⁺); RT=2.19 (UV, ELSD) 96%, 95%. ¹H NMR (DMSO-d₆): 1.19 (s, 9H), 3.00 (t, 2H), 3.51 (dt, 2H), 5.98 (br. s, NH), 6.97 (s, 1H), 7.44 (s, 1H), 9.57 (s, 1H, NHCO).

### 2-(4-Fluorophenyl)-N-(6-nitro-2,3-dihydro-1H-indol-5-yl)-acetamide

LC/MS (m/z) 315.0 ([M]⁺); RT=2.33 (UV, ELSD) 87%, 99%. ¹H NMR (DMSO-d₆): 2.99 (t, 2H), 3.49 (dt, 2H), 3.62 (s, 2H), 6.00 (br. s, NH), 6.91 (s, 1H), 7.15 (t, 2H), 7.28 (s, 1H), 7.33 (dd, 2H), 9.95 (s, 1H, NHCO).

### [6-Nitro-2,3-dihydro-1H-indol-5-yl]-carbamic acid ethyl ester

LC/MS (m/z) 250.9 ([M]⁺); RT=1.92 (UV, ELSD) 93%, 98%. ¹H NMR (DMSO-d₆): 1.19 (t, 3H), 2.99 (t, 2H), 3.50 (dt, 2H), 4.06 (q, 2H), 5.96 (br. s, NH), 6.92 (s, 1H), 7.24 (s, 1H), 9.22 (s, 1H, NHCO).

### [6-Nitro-2,3-dihydro-1H-indol-5-yl]-carbamic acid propyl ester

LC/MS (m/z) 264.9 ([M]⁺); RT=2.36 (UV, ELSD) 93%, 99%. ¹H NMR (DMSO-d₆): 0.89 (t, 3H), 1.59 (m, 2H), 2.99 (t, 2H), 3.50 (t, 2H), 3.97 (t, 2H), 5.96 (br. s, NH), 6.92 (s, 1H), 7.24 (s, 1H), 9.22 (s, 1H, NHCO).

### 3,3-Dimethyl-N-(6-bromo-2,3-dihydro-1H-indol-5-yl)-butyramide

To a stirred solution of 3,3-dimethyl-N-[1-(2,2,2-trifluoroacetyl)-2,3-dihydro-1H-indol-5-yl]-butyramide (0.624 g, 1.90 mmol) in acetic acid (20 ml) bromine (0.195 ml, 1 eq.) was added. After 45 min more bromine (0.195 ml) was added. The reaction mixture was poured into soution of Na₂SO₃ (5 g) in water (100 ml). The product N-[6-bromo-1-(2,2,2-trifluoro-acetyl)-2,3-dihydro-1H-indol-5-yl]-3,3-dimethylbutyramide was separated by filtration, washed with saturated aqueous NaHCO₃ and water to furnish 0.555 g of colorless solid. Yield 71%. LC/MS (m/z) 409.0 ([M+1]⁺); RT=3.38 (UV, ELSD) 97.5%, 85.5%. ¹H NMR (DMSO-d₆): 1.05 (s, 9H), 2.25 (s, 2H), 3.22 (t, 2H), 4.30 (t, 2H), 7.55 (s, 1H), 8.25 (s, 1H), 9.37 (s, 1H, NHCO).

The solid (100 mg) was redissolved in methanol (10 ml) followed by addition of K₂CO₃ (0.52 g) in water (5 ml). After stirring at 50° C. for 5 min, the reaction mixture was poured into ice/water mixture and the title compound was isolated by filtration to give 0.057 g of colorless solid. Yield 75%. LC/MS (m/z) 313.0 ([M+1]⁺); RT=1.71, (UV, ELSD) 97.5%, 98.9%.

Preparation of Intermediates of the General Formula XVIII

### N-(4-Chloro-2,3-dihydro-1H-indol-5-yl)-3,3-dimethylbutyramide

To a solution of crude N-[4-Chloro-1-(2,2,2-trifluoroacetyl)-2,3-dihydro-1H-indol-5-yl]-3,3-dimethylbutyramide (ca. 100 mg) in MeOH (10 ml) a solution of K₂CO₃ (0.5 g) in water (2 ml) was added. The obtained mixture was heated at 50° C. for 5 min and quenched with ethyl acetate and water. The organic solution was filtered via SiO₂ (5 g) and evaporated in vacuo to furnish 20 mg of the title compound. The crude product was used in the next step without purification. LC/MS (m/z) 267.1 ([M+1]⁺); RT=1.61 (UV, ELSD) 45%, 78%.

### N-(2,3-Dihydro-1H-indol-5-yl)-2-(4-fluoro-phenyl)-acetamide

To a solution of 2-(4-Fluorophenyl)-N-[1-(2,2,2-trifluoroacetyl)-2,3-dihydro-1H-indol-5-yl]-acetamide (1.3 g, 3.55 mmol) in methanol (50 ml) K₂CO₃ (7.6 g) in water (20 ml) was added. The reaction mixture was kept at 50° C. for 5 min, poured into water and the title compound was separated by filtration. Yield 0.742 g, 77.4%. LC/MS (m/z) 271.0 ([M+1]⁺); RT=1.42, (UV, ELSD) 94.5%, 98.7%. ¹H NMR (DMSO-d₆): 2.85 (t, 2H), 3.36 (t, 2H), 3.55 (s, 2H); 5.28 (br, 1H, NH); 6.41 (d, 1H); 7.06 (dd, 1H); 7.12 (t, 2H); 7.28 (d, 1H); 7.35 (dd, 2H); 9.75 (s, 1H, NHCO).

The following compound was prepared analogously from 3,3-dimethyl-N-[1-(2,2,2-trifluoroacetyl)-2,3-dihydro-1H-indol-5-yl]-butyramide:

### N-(2,3-Dihydro-1H-indol-5-yl)-3,3-dimethyl-butyramide

LC/MS (m/z) 232.9 ([M+1]⁺); RT=1.43, (UV, ELSD) 94.4%, 88.1%. ¹H NMR (DMSO-d₆): 1.00 (s, 9H); 2.09 (s, 2H); 2.84 (t, 2H); 3.37 (t, 2H); 5.25 (br, 1H, NH); 6.41 (d, 1H); 7.02 (dd, 1H); 7.29 (d, 1H); 9.34 (s, 1H, NHCO).

Compounds of the Invention

### Example 1

### 1a N-[4-Chloro-1-(4-trifluoromethylbenzyl)-2,3-dihydro-1H-indol-5-yl]-3,3-dimethylbutyramide

To a solution of N-(4-chloro-2,3-dihydro-1H-indol-5-yl)-3,3-dimethylbutyramide (10 mg), 4-trifluomethylbenzaldehyde (0.06 ml) and acetic acid (0.03 ml) in methanol (0.3 ml) NaBH₃CN (100 mg) was added. After 60 min the reaction mixture was partitioned between ethyl acetate and saturated aqueous NaHCO₃ solution. The organic layer was filtered via plug of SiO₂ (2 g), evaporated and purified by preparative LC/MS to give 11 mg of the title compound as colourless solid. LC/MS (m/z) 425.2 ([M+1]⁺); RT=4.01, (UV, ELSD) 95%, 99%. ¹H NMR (DMSO-d₆): 1.03 (s, 9H), 2.16 (s, 2H), 2.97 (t, 2H), 3.40 (t, 2H), 4.41 (s, 2H), 6.48 (d, 1H), 7.03 (d, 1H), 7.56 (d, 2H), 7.72 (d, 2H), 9.12 (s, 1H, NHCO).

The following compound was prepared analogously using 5-chloro-2-thiophenecarboxaldehyde:

### 1b N-[4-Chloro-1-(5-chlorothiophen-2-ylmethyl)-2,3-dihydro-1H-indol-5-yl]-3,3-dimethylbutyramide

LC/MS (m/z) 397.0 ([M+1]⁺); RT=3.91, (UV, ELSD) 97%, 99%. ¹H NMR (DMSO-d₆): 1.03 (s, 9H), 2.17 (s, 2H), 2.92 (t, 2H), 3.37 (t, 2H), 4.46 (s, 2H), 6.60 (d, 1H), 6.95 (d, 1H), 6.99 (d, 1H), 7.07 (d, 1H), 9.12 (s, 1H, NHCO).

### Example 2

### 2a [1-(4-Fluorobenzyl)-2,3-dihydro-1H-indol-5-yl]-carbamic acid propyl ester

To a cold (ice/water bath) solution of 1-(4-fluorobenzyl)-5-aminoindoline in acetonitrile (0.2 M, 0.15 ml) propyl chloroformiate (0.02 ml or ca. 20 mg) was added followed by pyridine (0.03 ml). The reaction mixture was allowed to stand at room temperature for 60 min and evaporated in vacuo. The title compound was separated by preparative LC/MS, yield 5.8 mg, 59%. LC/MS (m/z) 329.1 ([M+1]⁺); RT=2.68, (UV, ELSD) 94%, 99%.

The following compounds were obtained analogously from corresponding 5-aminoindolines and commercially available appropriate chloroformiates, carbamyl chlorides, sulphonyl chlorides, acid chlorides, di-tert-butyl dicarbonate (Boc₂O) or isocyanates, which are listed in the Table 1 below. Pyridine was used as a base in case of chloroformiates, carbamyl chlorides, and sulphonyl chlorides. Triethylamine was used as a base in case of acid chlorides. No base was used in case of isocyanates and Boc₂O:

### 2b N-[1-(4-Fluorobenzyl)-2,3-dihydro-1H-indol-5-yl]-C-phenyl-methanesulfonamide

LC/MS (m/z) 395.3 ([M−1]⁺); RT=3.17, (UV, ELSD) 80%, 100%.

### 2c 4-Fluoro-N-[1-(4-fluorobenzyl)-2,3-dihydro-1H-indol-5-yl]-benzamide

LC/MS (m/z) 365.4 ([M+1]⁺); RT=2.90, (UV, ELSD) 96%, 100%.

### 2d N-[1-(4-Fluorobenzyl)-2,3-dihydro-1H-indol-5-yl]-3,3-dimethylbutyramide

LC/MS (m/z) 341.1 ([M+1]⁺); RT=2.79, (UV, ELSD) 94%, 100%.

### 2e N-[1-(4-Fluorobenzyl)-2,3-dihydro-1H-indol-5-yl]-2-thiophen-2-ylacetamide

LC/MS (m/z) 367.1 ([M+1]⁺); RT=2.72, (UV, ELSD) 93%, 100%.

### 2f N-[1-(4-Fluorobenzyl)-2,3-dihydro-1H-indol-5-yl]-2-(4-fluorophenyl)-acetamide

LC/MS (m/z) 379.3 ([M+1]⁺); RT=2.82, (UV, ELSD) 95%, 100%.

### 2g 3-[1-(5-Chlorothiophen-2-ylmethyl)-2,3-dihydro-1H-indol-5-yl]-1,1-diisopropylurea

LC/MS (m/z) 392.3 ([M+1]⁺); RT=3.14, (UV, ELSD) 75%, 89%.

### 2h Morpholine-4-carboxylic acid [1-(5-chlorothiophen-2-ylmethyl)-2,3-dihydro-1H-indol-5-yl]-amide

LC/MS (m/z) 378.2 ([M+1]⁺); RT=2.33, (UV, ELSD) 97%, 100%.

### 2i Pyrrolidine-1-carboxylic acid [1-(5-chlorothiophen-2-ylmethyl)-2,3-dihydro-1H-indol-5-yl]-amide

LC/MS (m/z) 362.0 ([M+1]⁺); RT=2.48, (UV, ELSD) 83%, 99%.

### 2j [1-(5-Chlorothiophen-2-ylmethyl)-2,3-dihydro-1H-indol-5-yl]-carbamic acid 2-benzyloxyethyl ester

LC/MS (m/z) 442.1 ([M]⁺); RT=3.52, (UV, ELSD) 62%, 86%.

### 2k 3-[1-(5-Chlorothiophen-2-ylmethyl)-2,3-dihydro-1H-indol-5-yl]-1-methyl-1-propylurea

LC/MS (m/z) 364.3 ([M+1]⁺); RT=2.73, (UV, ELSD) 94%, 100%.

### 2l [1-(5-Chlorothiophen-2-ylmethyl)-2,3-dihydro-1H-indol-5-yl]-carbamic acid tert-butyl ester

LC/MS (m/z) 364.3 ([M]⁺); RT=3.50, (UV, ELSD) 97%, 100%.

### 2m N-[1-(5-Chlorothiophen-2-ylmethyl)-2,3-dihydro-1H-indol-5-yl]-C-phenyl-methanesulfonamide

LC/MS (m/z) 418.2 ([M]⁺); RT=3.44, (UV, ELSD) 98%, 100%.

### 2n Butane-1-sulfonic acid [1-(5-chlorothiophen-2-ylmethyl)-2,3-dihydro-1H-indol-5-yl]-amide

LC/MS (m/z) 384.1 ([M]⁺); RT=3.43, (UV, ELSD) 98%, 100%.

### 2o N-[1-(5-Chlorothiophen-2-ylmethyl)-2,3-dihydro-1H-indol-5-yl]-4-fluorobenzamide

LC/MS (m/z) 386.0 ([M]⁺); RT=3.35, (UV, ELSD) 91%, 100%.

### 2p N-[1-(5-Chlorothiophen-2-ylmethyl)-2,3-dihydro-1H-indol-5-yl]-2,2-dimethylpropionamide

LC/MS (m/z) 349.0 ([M+1]⁺); RT=3.21, (UV, ELSD) 94%, 100%.

### 2q N-[1-(5-Chlorothiophen-2-ylmethyl)-2,3-dihydro-1H-indol-5-yl]-2-phenoxyacetamide

LC/MS (m/z) 398.0 (M); RT=3.46, (UV, ELSD) 80%, 100%.

### 2r N-[1-(5-Chlorothiophen-2-ylmethyl)-2,3-dihydro-1H-indol-5-yl]-3,3-dimethylbutyramide

LC/MS (m/z) 362.1 ([M]⁺); RT=3.34, (UV, ELSD) 84%, 99%.

### 2s N-[1-(5-Chlorothiophen-2-ylmethyl)-2,3-dihydro-1H-indol-5-yl]-butyramide

LC/MS (m/z) 335.0 ([M+1]⁺); RT=2.95, (UV, ELSD) 78%, 99%.

### 2t Cyclopentanecarboxylic acid [1-(5-chlorothiophen-2-ylmethyl)-2,3-dihydro-1H-indol-5-yl]-amide

LC/MS (m/z) 361.1 ([M+1]⁺); RT=3.22, (UV, ELSD) 84%, 99%.

### 2u N-[1-(5-Chlorothiophen-2-ylmethyl)-2,3-dihydro-1H-indol-5-yl]-2-thiophen-2-ylacetamide

LC/MS (m/z) 388.1 ([M]⁺); RT=3.22, (UV, ELSD) 76%, 98%.

### 2v N-[1-(5-Chlorothiophen-2-ylmethyl)-2,3-dihydro-NH-indol-5-yl]-isonicotinamide

LC/MS (m/z) 370.0 ([M+1]⁺); RT=2.22, U, ELSD) 96%, 100%.

### 2w N-[1-(5-Chlorothiophen-2-ylmethyl)-2,3-dihydro-1H-indol-5-yl]-4-dimethylaminobenzamide

LC/MS (m/z) 412.0 ([M+1]⁺); RT=3.09, (UV, ELSD) 87%, 100%.

### 2x N-[1-(5-Chlorothiophen-2-ylmethyl)-2,3-dihydro-1H-indol-5-yl]-2-(4-fluorophenyl)-acetamide

LC/MS (m/z) 401.0 ([M+1]⁺); RT=3.31, (UV, ELSD) 84%, 100%.

### 2y N-[1-(5-Chlorothiophen-2-ylmethyl)-2,3-dihydro-1H-indol-5-yl]-6-trifluoromethylnicotinamide

LC/MS (m/z) 437.1 ([M]⁺); RT=3.46, (UV, ELSD) 90%, 99%.

### 2z 1-tert-Butyl-3-[1-(5-chlorothiophen-2-ylmethyl)-2,3-dihydro-1H-indol-5-yl]-urea

LC/MS (m/z) 364.3 ([M+1]⁺); RT=2.80, (UV, ELSD) 97%, 100%.

### 2aa 1-[1-(5-Chlorothiophen-2-ylmethyl)-2,3-dihydro-1H-indol-5-yl]-3-ethylurea

LC/MS (m/z) 335.1 ([M]⁺); RT=2.34, (UV, ELSD) 96%, 100%.

### 2ab 1-Benzyl-3-[1-(5-chlorothiophen-2-ylmethyl)-2,3-dihydro-1H-indol-5-yl]-urea

LC/MS (m/z) 398.2 ([M+1]⁺); RT=2.85, (UV, ELSD) 84%, 100%.

### 2ac 1-[1-(5-Chlorothiophen-2-ylmethyl)-2,3-dihydro-1H-indol-5-yl]-3-phenethylurea

LC/MS (m/z) 411.9 ([M+1]⁺); RT=3.00, (UV, ELSD) 87%, 97%.

### 2ad 1-[1-(5-Chlorothiophen-2-ylmethyl)-2,3-dihydro-1H-indol-5-yl]-3-thiophen-2-ylurea

LC/MS (m/z) 390.0 ([M+1]⁺); RT=3.01, (UV, ELSD) 94%, 92%.

### 2ae 1-[1-(5-Chlorothiophen-2-ylmethyl)-2,3-dihydro-1H-indol-5-yl]-3-thiophen-3-ylurea

LC/MS (m/z) 390.2 ([M+1]⁺); RT=2.98, (UV, ELSD) 96%, 100%.

### 2af [1-(5-Chlorothiophen-2-ylmethyl)-2,3-dihydro-1H-indol-5-yl]-carbamic acid propyl ester

LC/MS (m/z) 351.2 ([M]⁺); RT=3.48, (UV, ELSD) 94.0%, 98.0%. ¹H NMR (DMSO-d₆): 0.92 (t, 3H); 1.62 (sextet, 2H); 2.84 (t, 2H); 3.22 (t, 2H), 3.89 (t, 2H), 4.38 (s, 2H), 6.59 (d, 1H); 6.92 (d, 1H); 6.98 (d, 1H); 7.07 (br. d (dd), 1H); 7.18 (br. s, 1H); 9.20 (br s, 1H, NH).

TABLE 1
Chloroformiates, sulphonyl chlorides, carbamyl chlorides, acid chlorides, and
isocyanates used in the preparation of compounds of the invention 2a-2af

| Compound of | | | | Catalog |
|---|---|---|---|---|
| the invention | Reagent | MW | Supplier | number |
| 2a | n-PROPYL CHLOROFORMATE | 122.55 | Aldrich | 24,946-7 |
| 2b | ALPHA-TOLUENESULFONYL CHLORIDE | 190.649 | Aldrich | 15,971-9 |
| 2c | 4-FLUOROBENZOYL CHLORIDE | 158.559 | Aldrich | 11,994-6 |
| 2d | TERT-BUTYLACETYL CHLORIDE | 134.605 | Aldrich | B8,880-2 |
| 2e | THIOPHENE-2-ACETYL CHLORIDE | 160.624 | Aldrich | 19,599-5 |
| 2f | 4-FLUOROPHENYLACETYL CHLORIDE | 172.585 | Aldrich | 46,695-6 |
| 2g | DIISOPROPYLCARBAMYL CHLORIDE | 163.647 | Aldrich | S31,027-1 |
| 2h | MORPHOLINE-4-CARBONYL CHLORIDE | 149.576 | Aldrich | 34,829-5 |
| 2i | 1-PYRROLIDINECARBONYL CHLORIDE | 133.577 | Aldrich | 20,635-0 |
| 2j | 2-BENZYLOXYETHYL CHLOROFORMATE | 214.647 | Aldrich | 52,514-6 |
| 2k | N-ISOPROPYL-N-METHYL-CARBAMYL CHLORIDE | 135.59 | Lundbeck | C0005221 |
| 2l | DI-TERT-BUTYL DICARBONATE | 218.247 | Fluka | 34660 |
| 2m | ALPHA-TOLUENESULFONYL CHLORIDE | 190.649 | Aldrich | 15,971-9 |
| 2n | 1-BUTANESULFONYL CHLORIDE | 156.632 | Aldrich | 26,360-5 |
| 2o | 4-FLUOROBENZOYL CHLORIDE | 158.559 | Aldrich | 11,994-6 |
| 2p | PIVALOYL CHLORIDE | 120.578 | Aldrich | T7,260-5 |
| 2q | PHENOXYACETYL CHLORIDE | 170.594 | Aldrich | 15,862-3 |
| 2r | TERT-BUTYLACETYL CHLORIDE | 134.605 | Aldrich | B8,880-2 |
| 2s | BUTYRYL CHLORIDE | 106.551 | Aldrich | 10,961-4 |
| 2t | CYCLOPENTANECARBONYL CHLORIDE | 132.589 | Aldrich | 32,831-6 |
| 2u | THIOPHENE-2-ACETYL CHLORIDE | 160.624 | Aldrich | 19,599-5 |
| 2v | ISONICOTINOYL CHLORIDE HYDROCHLORIDE | 178.018 | Aldrich | 22,875-3 |
| 2w | 4-DIMETHYLAMINOBENZOYL CHLORIDE | 183.637 | Aldrich | 52,611-8 |
| 2x | 4-FLUOROPHENYLACETYL CHLORIDE | 172.585 | Aldrich | 46,695-6 |
| 2y | 6-(TRIFLUOROMETHYL)NICOTINOYL CHLORIDE | 209.554 | Fluorochem | 9368 |
| 2z | TERT-BUTYL ISOCYANATE | 99.132 | Aldrich | 14,445-2 |
| 2aa | ETHYL ISOCYANATE | 71.08 | Aldrich | E3,330-0 |
| 2ab | BENZYL ISOCYANATE | 133.149 | Aldrich | 22,726-9 |
| 2ac | PHENETHYL ISOCYANATE | 147.176 | Aldrich | 45,617-9 |
| 2ad | 2-THIENYL ISOCYANATE | 125.151 | Maybridge | CC 13006 |
| 2ae | 3-THIENYL ISOCYANATE | 125.151 | Maybridge | CC 13106 |
| 2af | n-PROPYL CHLOROFORMATE | 122.55 | Aldrich | 24,946-7 |

### Example 3

### 3a 2,2-Dimethyl-N-[6-nitro-1-(4-trifluoromethylbenzyl)-2,3-dihydro-1H-indol-5-yl]-propionamide

To a stirred solution of 2,2-Dimethyl-N-(6-nitro-2,3-dihydro-1H-indol-5-yl)-propionamide (0.379 g, 1.44 mmol) in methanol (25 ml) 4 trifluoromethylbenzaldehyde (0.8 ml), acetic acid (0.8 ml) and a solution of NaBH₃CN (0.8 g) in methanol (10 ml) were added in 4 portions during 3 hours until reaction completion. The obtained reaction mixture was concentrated in vacuo to a small volume, quenched with saturated aqueous NaHCO₃ solution, and sonicated for several minutes. The title compound was separated by filtration to give 0.574g of red solid, yield 95%. LC/MS (m/z) 422.1 ([M+1]⁺); RT=4.11, (UV, ELSD) 96%, 99%. ¹H NMR (DMSO-d₆): 1.20 (s, 9H), 3.04 (t, 2H), 3.42 (t, 2H), 4.49 (s, 2H), 7.09 (s, 1H), 7.48 (s, 1H), 7.57 (d, 2H), 7.73 (d, 2H), 9.62 (s, 1H, NHCO).

The following compounds were prepared analogously:

### 3b N-[1-(5-Chlorothiophen-2-ylmethyl)-6-nitro-2,3-dihydro-1H-indol-5-yl]-2,2-dimethylpropionamide

LC/MS (m/z) 394.0 ([M+1]⁺); RT=4.07, (UV, ELSD) 97%, 98%. ¹H NMR (DMSO-d₆): 1.20 (s, 9H), 3.00 (t, 2H), 3.41 (t, 2H), 4.55 (s, 2H), 6.97 (d, 1H), 7.01 (d, 1H), 7.22 (s, 1H), 7.47 (s, 1H), 9.62 (s, 1H, NHCO).

### 3c 2-(4-Fluorophenyl)-N-[6-nitro-1-(4-trifluoromethylbenzyl)-2,3-dihydro-1H-indol-5-yl]-acetamide

LC/MS (m/z) 474.2 ([M+1]⁺); RT=3.89, (UV, ELSD) 80%, 97%.

### 3d N-[1-(5-Chlorothiophen-2-ylmethyl)-6-nitro-2,3-dihydro-1H-indol-5-yl]-2-(4-fluorophenyl)-acetamide

LC/MS (m/z) 445.1 ([M]⁺); RT=3.88, (UV, ELSD) 79.8%, 98.9%. ¹H NMR (DMSO-d₆): 2.98 (t, 2H); 3.41 (t, 2H), 3.63 (s, 2H), 4.54 (s, 2H), 6.96 (d, 1H); 7.0 (d, 1H); 7.16 (t, 2H); 7.17 (s, 1H); 7.31 (s, 1H); 7.34 (dd, 2H); 10.03 (s, 1H, NH).

### 3e N-[1-(5-Chlorothiophen-2-ylmethyl)-6-nitro-2,3-dihydro-1H-indol-5-yl]-3,3-dimethylbutyramide

LC/MS (m/z) 407.1 ([M]⁺); RT=4.07, (UV, ELSD) 72.4%, 98.7%. ¹H NMR (DMSO-d₆): 1.01 (s, 9H); 2.16 (s, 2H); 2.99 (t, 2H); 3.41 (t, 2H), 4.54 (s, 2H), 6.98 (d, 1H); 7.01 (d, 1H); 7.14 (s, 1H); 7.23 (s, 1H); 9.76 (s, 1H, NM.

### Example 4

### 4a N-[6-Amino-1-(5-chlorothiophen-2-ylmethyl)-2,3-dihydro-1H-indol-5-yl]-3,3-dimethylbutyramide

To a solution of 3,3-dimethyl-N-(6-nitro-2,3-dihydro-1H-indol-5-yl)-butyramide (15 mg), 5-chloro-2-thiophenecarboxaldehyde (50 mg), and acetic acid (0.1 ml) in methanol (5 ml) NaBH₃CN (200 mg) was added. After 30 min the reaction mixture was concentrated in vacuo to a small volume and partitioned between ethyl acetate and water. The organic solution was washed with aqueous HCl (1 M) and saturated aqueous NaHCO₃ and evaporated in vacuo.

The obtained residue was dissolved in tetrahydrofuran (10 ml) and acetic acid (2 ml) followed by addition of Zn powder (1 g). The obtained suspension was sonicated for 5 min, more Zn powder was added (0.5 g) and sonication continued for 2 min. The obtained suspension was filtered via a plug of SiO₂ (2 g), evaporated, and the title compound was separated by preparative LC/MS to give 6.5 mg of colourless solid, yield 32%. LC/MS (m/z) 378.0 ([M+1]⁺); RT=2.36, (UV, ELSD) 93%, 98%. %. ¹H NMR (DMSO-d₆): 1.03 (s, 9H), 2.17 (s, 2H), 2.82 (t, 2H), 3.30 (t, 2H), 3.55 (very br. s, NH₂ and H₂O), 4.38 (s, 2H), 6.27 (s, 1H), 6.81 (s, 1H), 6.93 (d, 1H), 7.00 (d, 1H), 9.27 (br. s, 1H, NHCO).

### 4b N-[6-Amino-1-(4-trifluoromethylbenzyl)-2,3-dihydro-1H-indol-5-yl]-2,2-dimethylpropionamide

The title compound was prepared from the above 2,2-dimethyl-N-[6-nitro-1-(4-trifluoromethylbenzyl)-2,3-dihydro-1H-indol-5-yl]-propionamide (see example 3) by reduction with Zn powder as described above for 1-(5-Chlorothiophen-2-ylmethyl)-5-aminoindoline (see preparation of intermediates of the general formula XXI). The crude solid residue after filtration via SiO₂ was treated with ethyl acetate and heptane and the title compound was separated by filtration. Yield 0.375 g, 71%, colourless solid. LC/MS (m/z) 392.3 ([M+1]⁺); RT=2.38, (UV, ELSD) 97%, 99%. ¹H NMR (DMSO-d₆): 1.20 (s, 9H), 2.77 (t, 2H), 3.25 (t, 2H), 4.28 (s, 2H), 4.34 (s, 2H, NH₂), 5.96 (s, 1H), 6.64 (s, 1H), 7.56 (d, 2H), 7.71 (d, 2H), 8.47 (s, 1H, NHCO).

The following compounds were prepared analogously from corresponding 6-nitroindolines of the general formula XXV in two steps via reductive alkylation with appropriate aldehyde as described in the example 3 followed by reduction with Zn powder as described above.

### 4c N-[6-Amino-1-(5-chlorothiophen-2-ylmethyl)-2,3-dihydro-1H-indol-5-yl]-2,2-dimethylpropionamide

LC/MS (m/z) 364.2 ([M+1]⁺); RT=2.19, (UV, ELSD) 98%, 99%. ¹H NMR (DMSO-d₆): 1.20 (s, 9H), 2.72 (t, 2H), 3.21 (t, 2H), 4.33 (s, 2H), 4.39 (br. s, 2H, NH₂), 6.07 (s, 1H), 6.64 (s, 1H), 6.91 (d, 1H), 6.98 (d, 1H), 8.48 (s, 1H, NHCO).

### 4d N-[6-Amino-1-(4-trifluoromethylbenzyl)-2,3-dihydro-1H-indol-5-yl]-2-(4-fluorophenyl)-acetamide

LC/MS (m/z) 444.0 ([M+1]⁺); RT=2.65, (UV, ELSD) 95%, 99%. ¹H NMR (DMSO-d₆): 2.76 (t, 2H), 3.24 (t, 2H), 3.57 (s, 2H), 4.26 (s, 2H), 4.51 (br. s, 2H, NH₂), 5.92 (s, 1H), 6.73 (s, 1H), 7.14 (t, 2H), 7.36 (dd, 2H), 7.55 (d, 2H), 7.71 (dd, 2H), 9.08 (s, 1H, NHCO).

### 4e N-[6-Amino-1-(4-trifluoromethylbenzyl)-2,3-dihydro-1H-indol-5-yl]-3,3-dimethylbutyramide

LC/MS (m/z) 406.0 ([M+1]⁺); RT=2.58, (UV, ELSD) 97.4%, 99.0%. ¹H NMR (DMSO-d₆): 1.02 (s, 9H); 2.12 (s, 2H); 2.78 (t, 2H); 3.24 (t, 2H); 4.28 (s, 2H), 4.48 (s, 2H, NH₂); 5.93 (s, 1H); 6.74 (s, 1H); 7.56 (d, 2H); 7.71 (d, 2H); 8.81 (s, 1H, NH).

### 4f N-[6-Amino-1-(4-fluorobenzyl)-2,3-dihydro-1H-indol-5-yl]-3,3-dimethylbutyramide

LC/MS (m/z) 356.0 ([M+1]⁺); RT=2.26, (UV, ELSD) 96.7%, 98.9%. ¹H NMR (DMSO-d₆): 1.02 (s, 9H); 2.13 (s, 2H); 2.73 (t, 2H); 3.18 (t, 2H); 4.16 (s, 2H), 4.48 (s, 2H, NH₂); 5.98 (s, 1H); 6.71 (s, 1H); 7.17 (t, 2H); 7.36 (dd, 2H); 8.80 (s, 1H, NH).

### 4g N-[6-Amino-1-(3-fluoro-4-trifluoromethylbenzyl)-2,3-dihydro-1H-indol-5-yl]-3,3-dimethylbutyramide

LC/MS (m/z) 424.0 ([M+1]⁺); RT=2.58, (UV, ELSD) 92.0%, 98.8%. ¹H NMR (DMSO-d₆): 1.01 (s, 9H); 2.12 (s, 2H); 2.79 (t, 2H); 3.28 (t, 2H); 4.27 (s, 2H), 4.48 (s, 2H, NH₂); 5.90 (s, 1H); 6.75 (s, 1H); 7.39 (d, 1H); 7.45 (d, 1H); 7.78 (t, 1H); 8.80 (s, 1H, NH.

### Example 5

### 5a N-[1-(5-Chlorothiophen-2-ylmethyl)-1H-indol-5-yl]-3,3-dimethylbutyramide

To a solution of N-[1-(5-Chlorothiophen-2-ylmethyl)-2,3-dihydro-1H-indol-5-yl]-3,3-dimethylbutyramide (20 mg) in dimethylsulfoxide-d₆ (0.6 ml) 2,3,5,6-tetrachloro-[1,4]benzoquinone (65 mg) was added. The obtained mixture was heated at 70° C. for 5 min, allowed to cool and poured into aqueous NaHSO₃ solution (1 g in 5 ml) followed by addition of 25% aqueous NH₃ (5 ml) and 10% aqueous NaOH (5 ml). The mixture was extracted with CH₂Cl₂ (3×10 ml), the combined organic solution was washed with water and 1M HCl, filtered via plug of SiO₂ (10 g) and eluted with ethyl acetate/heptane (1:1). The crude product after evaporation was purified by preparative LC/MS to give 5 mg of the title compound as colourless solid. LC/MS (m/z) 361.1 ([M+1]⁺); RT=3.43, (UV, ELSD) 96%, 99%. ¹H NMR (DMSO-d₆): 1.03 (s, 9H), 2.16 (s, 2H), 5.51 (s, 2H), 6.41 (d, 1H), 6.95 (d, 1H), 6.98 (d, 1H), 7.21 (dd, 1H), 7.41 (d, 1H), 7.44 (d, 1H), 7.87 (d, 1H), 9.60 (s, 1H, NHCO).

### Example 6

### 6a N-[6-Bromo-1-(4-trifluoromethylbenzyl)-2,3-dihydro-1H-indol-5-yl]-3,3-dimethylbutyramide

The title compound was prepared from 3,3-dimethyl-N-(6-bromo-2,3-dihydro-1H-indol-5-yl)-butyramide (25 mg) and 4-trifluoromethylbenzaldehyde (64 mg) as described in example 1. Yield 16.7 mg. LC/MS (m/z) 469.1 ([M+1]⁺); RT=3.99, (UV, ELSD) 97.4%, 95.1%.

The following compound was prepared analogously using appropriate aldehyde:

### 6b N-[6-Bromo-1-(5-chlorothiophen-2-ylmethyl)-2,3-dihydro-1H-indol-5-yl]-3,3-dimethylbutyramide

Yield 16.8 mg. LC/MS (m/z) 443.1 ([M+1]⁺); RT=3.92, (UV, ELSD) 96.3%, 94.9%.

### Example 7

General procedure: To a mixture of aldehyde (25 mg or 0.025 ml) and appropriate indoline (7 mg) in methanol (0.5 ml) NaBH₃CN (20 mg) in methanol (0.2 ml) was added followed by acetic acid (0.05 ml). The obtained solution or suspension was sonicated for 5 min and then kept at 50° C. for 60 min followed by evaporation in vacuo. The residue was dissolved in DMSO (2.5 ml) and water (100 ml). The product was isolated by preparative LC/MS.

The following compounds were prepared accordingly from either N-(2,3-Dihydro-1H-indol-5-yl)-3,3-dimethyl-butyramide (compounds 7a-7z) or from N-(2,3-Dihydro-1H-indol-5-yl)-2-(4-fluoro-phenyl)-acetamide (compounds 7aa-7aw) and the appropriate aldehyde (see Table 2 below):

### 7a N-[1-(4-Chlorobenzyl)-2,3-dihydro-1H-indol-5-yl]-3,3-dimethylbutyramide

LC/MS (m/z) 357.2 ([M+1]⁺); RT=3.17, (UV, ELSD) 77.1%, 97.2%.

### 7b 3,3-Dimethyl-N-[1-(4-trifluoromethylbenzyl)-2,3-dihydro-1H-indol-5-yl]-butyramide

LC/MS (m/z) 391.4 ([M+1]⁺); RT=3.44, (UV, ELSD) 91.8%, 99.6%.

### 7c N-[1-(4-Isopropylbenzyl)-2,3-dihydro-1H-indol-5-yl]-3,3-dimethylbutyramide

LC/MS (m/z) 365.4 ([M+1]⁺); RT=3.13, (UV, ELSD) 89.4%, 99.2%.

### 7d N-[1-(3-Fluoro-4-trifluoromethylbenzyl)-2,3-dihydro-1H-indol-5-yl]-3,3-dimethylbutyramide

LC/MS (m/z) 409.2 ([M+1]⁺); RT=3.60, (UV, ELSD) 69.6%, 98.2%.

### 7e N-[1-(6-Chlorobenzo[1,3]dioxol-5-ylmethyl)-2,3-dihydro-1H-indol-5-yl]-3,3-dimethylbutyramide

LC/MS (m/z) 401.0 ([M+1]⁺); RT=3.22, (UV, ELSD) 94.6%, 99.5%.

### 7f N-[1-(3,5-Dimethyl-1-phenyl-1H-pyrazol-4-ylmethyl)-2,3-dihydro-1H-indol-5-yl]-3,3-dimethylbutyramide

LC/MS (m/z) 417.2 ([M+1]⁺); RT=2.23, (UV, ELSD) 93.3%, 98.9%.

### 7g N-[1-(2-Chloro-5-trifluoromethylbenzyl)-2,3-dihydro-1H-indol-5-yl]-3,3-dimethylbutyramide

LC/MS (m/z) 425.2 ([M+1]⁺); RT=3.79, (UV, ELSD) 75.4%, 98.4%.

### 7h N-{1-[5-(4-Chlorophenoxy)-1,3-dimethyl-1H-pyrazol-4-ylmethyl]-2,3-dihydro-1H-indol-5-yl}-3,3-dimethylbutyramide

LC/MS (m/z) 467.3 ([M+1]⁺); RT=2.71, (UV, ELSD) 95.2%, 99.8%.

### 7j 3,3-Dimethyl-N-[1-(6-p-tolyloxy-pyridin-3-ylmethyl)-2,3-dihydro-1H-indol-5-yl]-butyramide

LC/MS (m/z) 430.2 ([M+1]⁺); RT=3.15, (UV, ELSD) 76.6%, 97.9%.

### 7k N-{1-[6-(4-Chlorophenylsulfanyl)-pyridin-3-ylmethyl]-2,3-dihydro-1H-indol-5-yl}-3,3-dimethylbutyramide

LC/MS (m/z) 466.0 ([M+1]⁺); RT=3.45, (UV, ELSD) 69.3%, 97.1%.

### 7l N-{1-[6-(4-Cyanophenoxy)-pyridin-3-ylmethyl-2,3-dihydro-1H-indol-5-yl}-3,3-dimethylbutyramide

LC/MS (m/z) 441.4 ([M+1]⁺); RT=2.98, (UV, ELSD) 79.8%, 98.9%.

### 7m 3,3-Dimethyl-N-[1-(6-trifluoromethylpyridin-3-ylmethyl)-2,3-dihydro-1H-indol-5-yl]-butyramide

LC/MS (m/z) 392.4 ([M+1]⁺); RT=3.10, (UV, ELSD) 82.5%, 99.5%.

### 7p 3,3-Dimethyl-N-[1-(3-methyl-benzo[b]thiophen-2-ylmethyl)-2,3-dihydro-1H-indol-5-yl]-butyramide

LC/MS (m/z) 393.3 ([M+1]⁺); RT=3.56, (UV, ELSD) 72.8%, 97.9%.

### 7q N-[1-(6-Fluoro-4H-benzo[1,3]dioxin-8-ylmethyl)-2,3-dihydro-1H-indol-5-yl]-3,3-dimethylbutyramide

LC/MS (m/z) 399.2 ([M+1]⁺); RT=2.75, V, ELSD) 84.9%, 99.3%.

### 7s 3,3-Dimethyl-N-[1-(6-phenoxypyridin-3-ylmethyl)-2,3-dihydro-1H-indol-5-yl]-butyramide

LC/MS (m/z) 416.2 ([M+1]⁺); RT=2.98, (UV, ELSD) 67.5%, 96.5%.

### 7u 3,3-Dimethyl-N-[1-(3-methyl-5-phenyl-isoxazol-4-ylmethyl)-2,3-dihydro-1H-indol-5-yl]-butyramide

LC/MS (m/z) 404.4 ([M+1]⁺); RT=3.24, (UV, ELSD) 97.6%, 99.9%.

### 7v N-(1-Benzo[b]thiophen-2-ylmethyl-2,3-dihydro-1H-indol-5-yl)-3,3-dimethylbutyramide

LC/MS (m/z) 379.3 ([M+1]⁺); RT=3.44, (UV, ELSD) 71.8%, 97.6%.

### 7w N-(1-(4-Fluorophenyl)-5-methyl-1H-pyrazol-4-ylmethyl]-2,3-dihydro-1H-indol-5-yl)-3,3-dimethylbutyramide

LC/MS (m/z) 421.4 ([M+1]⁺); RT=2.24, (UV, ELSD) 79.5%, 98.9%.

### 7y 3,3-Dimethyl-N-[1-(5-methylthiophen-2-ylmethyl)-2,3-dihydro-1H-indol-5-yl]-butyramide

LC/MS (m/z) 343.1 ([M+1]⁺); RT=2.84, (UV, ELSD) 59.9%, 89.1%.

### 7z 3,3-Dimethyl-N-[1-(4-pyrrol-1-yl-benzyl)-2,3-dihydro-1H-indol-5-yl]-butyramide

LC/MS (m/z) 388.3 ([M+1]⁺); RT=3.03, (UV, ELSD) 81.1%, 99.5%.

### 7aa N-[1-(4-Chlorobenzyl)-2,3-dihydro-1H-indol-5-yl]-2-(4-fluorophenyl)-acetamide

LC/MS (m/z) 395.1 ([M+1]⁺); RT=2.99, (UV, ELSD) 93.4%, 93.1%.

### 7ab 2-(4-Fluorophenyl)-N-[1-(4-trifluoromethylbenzyl)-2,3-dihydro-1H-indol-5-yl]-acetamide

LC/MS (m/z) 429.1 ([M+1]⁺); RT=3.24, (UV, ELSD) 76.6%, 87.4%.

### 7ac 2-(4-Fluorophenyl)-N-[1-(4-isopropylbenzyl)-2,3-dihydro-1H-indol-5-yl]-acetamide

LC/MS (m/z) 403.1 ([M+1]⁺); RT=2.96, (UV, ELSD) 91.5%, 83.7%.

### 7ad 2-(4-Fluorophenyl)-N-[1-(3-fluoro-4-trifluoromethylbenzyl)-2,3-dihydro-1H-indol-5-yl]-acetamide

LC/MS (m/z) 447.2 ([M+1]⁺); RT=3.36, (UV, ELSD) 79.3%, 90.4%.

### 7ae N-[1-(6-Chlorobenzo[1,3]dioxol-5-ylmethyl)-2,3-dihydro-1H-indol-5-yl]-2-(4-fluorophenyl)-acetamide

LC/MS (m/z) 439.0 ([M+1]⁺); RT=3.02, (UV, ELSD) 94.0%, 92.2%.

### 7af N-[1-(3,5-Dimethyl-1-phenyl-1H-pyrazol-4-ylmethyl)-2,3-dihydro-1H-indol-5-yl]-2-(4-fluorophenyl)-acetamide

LC/MS (m/z) 455.0 ([M+1]⁺); RT=2.14, (UV, ELSD) 97.1%, 91.4%.

### 7ag N-[1-(2-Chloro-5-trifluoromethylbenzyl)-2,3-dihydro-1H-indol-5-yl]-2-(4-fluorophenyl)-acetamide

LC/MS (m/z) 463.1 ([M+1]⁺); RT=3.53, (UV, ELSD) 98.3%, 95.3%.

### 7ah N-{1-[5-(4-Chlorophenoxy)-1,3-dimethyl-1H-pyrazol-4-ylmethyl]-2,3-dihydro-1H-indol-5-yl}-2-(4-fluorophenyl)-acetamide

LC/MS (m/z) 503.3 ([M+1]⁺); RT=2.55, (UV, ELSD) 92.3%, 90.5%.

### 7ai N-{1-[6-(4-Cyanophenoxy)-pyridin-3-ylmethyl]-2,3-dihydro-1H-indol-5-yl}-2-(4 fluorophenyl)-acetamide

LC/MS (m/z) 479.1 ([M+1]⁺); RT=2.81, (UV, ELSD) 84.1%, 87.1%.

### 7al 2-(4-Fluorophenyl)-N-[1-(3-methyl-benzo[b]thiophen-2-ylmethyl)-2,3-dihydro-1H-indol-5-yl]-acetamide

LC/MS (m/z) 431.2 ([M+1]⁺); RT=3.34, (UV, ELSD) 60.2%, 88.8%.

### 7am N-[1-(6-Fluoro-4H-benzo[1,3]dioxin-8-ylmethyl)-2,3-dihydro-1H-indol-5-yl]-2-(4-fluorophenyl)-acetamide

LC/MS (m/z) 437.0 ([M+1]⁺); RT=2.62, (UV, ELSD) 79.7%, 83.6%.

### 7ao 2-(4-Fluorophenyl)-N-[1-(6-phenoxypyridin-3-ylmethyl)-2,3-dihydro-1H-indol-5-yl]-acetamide

LC/MS (m/z) 454.3 ([M+1]⁺); RT=2.81, (UV, ELSD) 77.9%, 83.1%.

### 7ar N-(1-Benzo[b]thiophen-2-ylmethyl-2,3-dihydro-1H-indol-5-yl)-2-(4-fluorophenyl)-acetamide

LC/MS (m/z) 417.2 ([M+1]⁺); RT=3.22, (UV, ELSD) 80.2%, 89.4%.

### 7as 2-(4-Fluorophenyl)-N-{1-[1-(4-fluorophenyl)-5-methyl-1H-pyrazol-4-ylmethyl]-2,3-dihydro-1H-indol-5-yl}-acetamide

LC/MS (m/z) 459.3 ([M+1]⁺); RT=2.15, (UV, ELSD) 91.5%, 88.7%.

### 7au 2-(4-Fluorophenyl)-N-[1-(5-methylthiophen-2-ylmethyl)-2,3-dihydro-1H-indol-5-yl]-acetamide

LC/MS (m/z) 381.1 ([M+1]⁺); RT=2.71, (UV, ELSD) 76.6%, 82.9%.

### 7av 2-(4-Fluorophenyl)-N-[1-(4-pyrrol-1-yl-benzyl)-2,3-dihydro-1H-indol-5-yl]-acetamide

LC/MS (m/z) 426.1 ([M+1]⁺); RT=2.87, (UV, ELSD) 92.2%, 88.2%.

TABLE 2
Aldehydes used in the preparation of compounds of the invention 7a-7av

| Compound of | | | | Catalog |
|---|---|---|---|---|
| the invention | Aldehyde | MW | Supplier | number |
| 7a | 4-CHLOROBENZALDEHYDE | 140.568 | Aldrich | |
| 7b | 4-(TRIFLUOROMETHYL)-BENZALDEHYDE | 174.12 | Aldrich | |
| 7c | 4-ISOPROPYLBENZALDEHYDE | 148.204 | Aldrich | |
| 7d | 3-FLUORO-4-(TRIFLUOROMETHYL)-BENZALDEHYDE | 192.111 | ABCR | AV20008 |
| 7e | 6-CHLOROPIPERONAL | 184.577 | ABCR | AV13607 |
| 7f | 3,5-DIMETHYL-1-PHENYLPYRAZOLE-4-CARBOXALDEHYDE | 200.24 | Acros Organics | 40852-0050 |
| 7g | 2-CHLORO-5-(TRIFLUOROMETHYL)-BENZALDEHYDE | 208.566 | Aldrich | 37,682-5 |
| 7h | 5-(4-CHLOROPHENOXY)-1,3-DIMETHYL-1H-PYRAZOLE-4-CARBALDEHYDE | 250.684 | Bionet Research | 11F-431S |
| 7j | 6-(4-METHYLPHENOXY)-NICOTINALDEHYDE | 213.235 | Bionet Research | 5L-355S |
| 7k | 6-[(4-CHLOROPHENYL)SULFANYL]-NICOTINALDEHYDE | 249.72 | Bionet Research | 5L-356S |
| 7l | 4-[(5-FORMYL-2-PYRIDINYL)OXY]-BENZENECARBONITRILE | 224.218 | Bionet Research | 6L-309S |
| 7m | 6-(TRIFLUOROMETHYL)PYRIDINE-3-CARBOXALDEHYDE | 175.109 | Fluorochem | 9397 |
| 7p | 3-METHYLBENZO[B]THIOPHENE-2-CARBOXALDEHYDE | 176.238 | ABCR | AV11375 |
| 7q | 5-FLUORO-4H-1,3-BENZODIOXINE-5-CARBALDEHYDE | 182.149 | Maybridge | CC 01904 |
| 7s | 6-PHENOXYNICOTINALDEHYDE | 199.208 | Maybridge | CC 19604 |
| 7u | 3-METHYL-5-PHENYL-4-ISOXAZOLECARBALDEHYDE | 187.197 | Maybridge | CC 20304 |
| 7v | 1-BENZOTHIOPHENE-2-CARBALDEHYDE | 162.211 | Specs | 942/25034639 |
| 7w | 1-(4-FLUOROPHENYL)-5-METHYL-1H-PYRAZOLE-4-CARBALDEHYDE | • 204.203 | Maybridge | MO 00310 |
| 7y | 5-METHYL-2-THIOPHENECARBOXALDEHYDE | 126.178 | Aldrich | M8,441-0 |
| 7z | 4-(1H-PYRROL-1-YL)BENZALDEHYDE | 171.198 | Maybridge | N/A |
| 7aa | 4-CHLOROBENZALDEHYDE | 140.568 | Aldrich | |
| 7ab | 4-(TRIFLUOROMETHYL)-BENZALDEHYDE | 174.12 | Aldrich | |
| 7ac | 4-ISOPROPYLBENZALDEHYDE | 148.204 | Aldrich | |
| 7ad | 3-FLUORO-4-(TRIFLUOROMETHYL)-BENZALDEHYDE | 192.111 | ABCR | AV20008 |
| 7ae | 6-CHLOROPIPERONAL | 184.577 | ABCR | AV13607 |
| 7af | 3,5-DIMETHYL-1-PHENYLPYRAZOLE-4-CARBOXALDEHYDE | 200.24 | Acros Organics | 40852-0050 |
| 7ag | 2-CHLORO-5-(TRIFLUOROMETHYL)BENZALDEHYDE | 208.566 | Aldrich | 37,682-5 |
| 7ah | 5-(4-CHLOROPHENOXY)-1,3-DIMETHYL-1H-PYRAZOLE-4-CARBALDEHYDE | 250.684 | Bionet Research | 11F-431S |
| 7ai | 4-[(5-FORMYL-2-PYRIDINYL)OXY]BENZENECARBONITRILE | 224.218 | Bionet Research | 6L-309S |
| 7al | 3-METHYLBENZO[B]THIOPHENE-2-CARBOXALDEHYDE | 176.238 | ABCR | AV11375 |
| 7am | 5-FLUORO-4H-1,3-BENZODIOXINE-5-CARBALDEHYDE | 182.149 | Maybridge | CC 01904 |
| 7ao | 6-PHENOXYNICOTINALDEHYDE | 199.208 | Maybridge | CC 19604 |
| 7ar | 1-BENZOTHIOPHENE-2-CARBALDEHYDE | 162.211 | Specs | 942/25034639 |
| 7as | 1-(4-FLUOROPHENYL)-5-METHYL-1H-PYRAZOLE-4-CARBALDEHYDE | 204.203 | Maybridge | MO 00310 |
| 7au | 5-METHYL-2-THIOPHENECARBOXALDEHYDE | 126.178 | Aldrich | M8,441-0 |
| 7av | 4-(1H-PYRROL-1-YL)BENZALDEHYDE | 171.198 | Maybridge | N/A |

In Vitro and In Vivo Testing

The compounds of the invention have been tested and shown effect in one or more of the below models:

Relative Efflux Through the KCNQ2 Channel.

This exemplifies a KCNQ2 screening protocol for evaluating compounds of the present invention. The assay measures the relative efflux through the KCNQ2 channel, and was carried out according to a method described by Tang et al. (Tang, W. et. al., J. Biomol. Screen. 2001, 6, 325-331) for hERG potassium channels with the modifications described below.

An adequate number of CHO cells stably expressing voltage-gated KCNQ2 channels were plated at a density sufficient to yield a mono-confluent layer on the day of the experiment. Cells were seeded on the day before the experiment and loaded with 1 μCi/ml [⁸⁶Rb] over night. On the day of the experiment cells were washed with a HBSS-containing buffer. Cells were pre-incubated with drug for 30 minutes and the ⁸⁶Rb⁺ efflux was stimulated by a submaximal concentration of 15 mM KCl in the continued presence of drug for additional 30 minutes. After a suitable incubation period, the supernatant was removed and counted in a liquid scintillation counter (Tricarb). Cells were lysed with 2 mM NaOH and the amount of ⁸⁶Rb+ was counted. The relative efflux was calculated ((CPMₛᵤₚₑᵣ(CPMₛᵤₚₑᵣ+CPM_{cell}))_{Cmpd}/(CPMₛᵤₚₑᵣ/(CPMₛᵤₚₑᵣ+CPM_{cell}))_{15mM KCl})*100−100.

The compounds of the invention have an EC₅₀ of less than 20000 nM, in most cases less than 2000 nM and in many cases less than 200 nM. Accordingly, the compounds of the invention are considered to be useful in the treatment of diseases associated with the KCNQ family potassium channels.

Electrophysiological Patch-Clamp Recordings.

Voltage-activated KCNQ2 currents were recorded from mammalian CHO cells by use of conventional patch-clamp recordings techniques in the whole-cell patch-clamp configuration (Hamill O P et.al. Pfügers Arch 1981; 391: 85-100). CHO cells with stable expression of voltage-activated KCNQ2 channels were grown under normal cell culture conditions in CO₂ incubators and used for electrophysiological recordings 1-7 days after plating. KCNQ2 potassium channels were activated by voltage steps up to +80 mV in increments of 5-20 mV (or with a ramp protocol) from a membrane holding potential between −100 mV and −40 mV (Tatulian L et al. J Neuroscience 2001; 21 (15): 5535-5545). The electrophysiological effects induced by the compounds were evaluated on various parameters of the voltage-activated KCNQ2 current. Especially effects on the activation threshold for the current and on the maximum induced current were studied.

Some of the compounds of the invention have been tested in this test. A left-ward shift of the activation threshold or an increase in the maximum induced potassium current is expected to decrease the activity in neuronal networks and thus make the compounds useful in diseases with increased neuronal activity—like epilepsia.

Maximum Electroshock

The test was conducted in groups of male mice using corneal electrodes and administering a square wave current of 26 mA for 0.4 seconds in order to induce a convulsion characterised by a tonic hind limb extension (Wlaz et al. Epilepsy Research 1998, 30, 219-229).

Pilocarpine Induced Seizures

Pilocarpine induced seizures are induced by intraperitoneal injection of pilocarpine 250 mg/kg to groups of male mice and observing for seizure activity resulting in loss of posture within a period of 30 minutes (Starr et al. Pharmacology Biochemistry and Behavior 1993, 45, 321-325).

Electrical Seizure-Threshold Test

A modification of the up-and-down method (Kimball et al. Radiation Research 1957, 1-12) was used to determine the median threshold to induce tonic hind-limb extension in response to corneal electroshock in groups of male mice. The first mouse of each group received an electroshock at 14 mA, (0.4 s, 50 Hz) and was observed for seizure activity. If a seizure was observed the current was reduced by 1 mA for the next mouse, however, if no seizure was observed then the current was increased by 1 mA. This procedure was repeated for all 15 mice in the treatment group.

Chemical Seizure-Threshold Test

The threshold dose of pentylenetetrazole required to induce a clonic convulsion was measured by timed infusion of pentylenetetrazole (5 mg/mL at 0.5 mL/minute) into a lateral tail vein of groups of male mice (Nutt et al. J Pharmacy and Pharmacology 1986, 38, 697-698).

Amygdala Kindling

Rats underwent surgery to implantation of tri-polar electrodes into the dorsolateral amygdala. After surgery the animals were allowed to recover before the groups of rats received either varying doses of test compound or the drug's vehicle. The animals were stimulated with their initial after discharge threshold +25 μA daily for 3-5 weeks and on each occasion seizure severity, seizure duration, and duration of electrical after discharge were noted. (Racine. Electroencephalography and Clinical Neurophysiology 1972, 32, 281-294).

Side Effects

Central nervous system side-effects were measured by measuring the time mice would remain on rotarod apparatus (Capacio et al. Drug and Chemical Toxicology 1992, 15, 177-201); or by measuring their locomotor activity by counting the number of infra-red beams crossed in a test cage (Watson et al. Neuropharmacology 1997, 36, 1369-1375). Hypothermic actions on the animals core body temperature of the compound were measured by either rectal probe or implanted radiotelemetry transmitters capable of measuring temperature (Keeney et al. Physiology and Behaviour 2001, 74, 177-184).

Pharmacokinetics

The pharmacokinetic properties of the compounds were determined via. i.v. and p.o. dosing to Spraque Dawley rats, and, thereafter, drawing blood samples over 20 hours. Plasma concentrations were determined with LC/MS/MS.

1. A compound of formula I 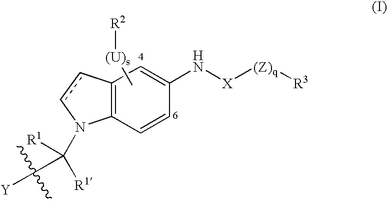
wherein
the dotted line represents an optional bond;
R¹ and R^{1′} are independently selected from the group consisting of hydrogen, C₁₋₆-alk(en/yn)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹ and R^{1′} together with the carbon atom to which they are attached form a 3-8 membered saturated or unsaturated ring which optionally contains 1 or 2 heteroatoms;
s is 0 or 1;
U is O, NR¹¹, S, SO₂, SO₂NR¹¹, CO—O or CO—NR¹¹; wherein R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or R² and R¹¹ together with the nitrogen atom to which they are attached form a 4-8 membered saturated or unsaturated ring which optionally contains 1, 2 or 3 further heteroatoms;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹⁰ and R^{10′} together with the nitrogen atom to which they are attached form a 4-8 membered saturated or unsaturated ring which optionally contains 1, 2 or 3 further heteroatoms;
with the proviso that when R² is NO₂, halogen or cyano then s is 0; and
with the proviso that when R² is a hydrogen atom or acyl and s is 1 then U is NR¹¹, O or S;
wherein the group —(U)ₛ—R² is linked to position 4 or 6 of the indole or indoline;
q is 0 or 1;
Z is O or S;
X is CO or SO₂; with the proviso that q is 0 when X is SO₂;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl and —NR¹²R^{12′}, optionally substituted NR¹²R^{12′}—C₁₋₆-alk(en/yn)yl, optionally substituted NR¹²R^{12′}—C₃₋₈-cycloalk(en)yl, optionally substituted NR¹²R^{12′}—C₃₋₈-cycloalk(en)yl-C₁₋₆-alk(en/yn)yl; wherein
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹² and R^{12′} together with the nitrogen atom to which they are attached form a 4-8 membered saturated or unsaturated ring which optionally contains 1, 2 or 3 further heteroatoms;
with the proviso that when R³ is NR¹²R^{12′} then q is 0;
and
Y represents a group of formula II, III, IV, V, VI, XXX or XXXI: 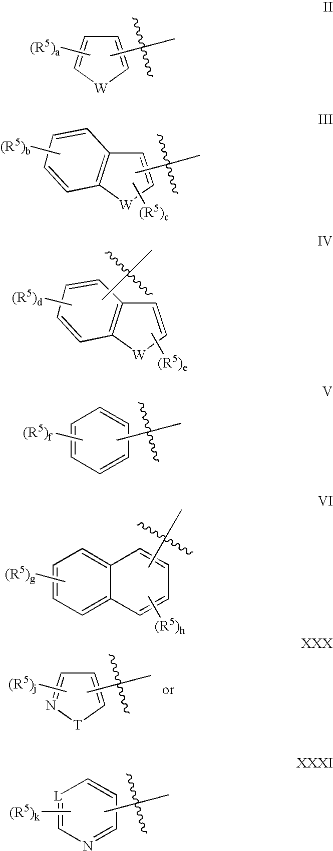
W is O or S;
T is N, NH or O;
L is N, C or CH;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1, 2 or 3; with the proviso that when T is a nitrogen atom then j is 0, 1, 2 or 3; and when T is NH or an oxygen atom then j is 0, 1 or 2;
k is 0, 1, 2, 3 or 4; and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸, or
two adjacent R⁵ together with the aromatic group to which they are attached form a 4-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl;
and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′} then R⁵ is not —S—R⁸;
or salts thereof;
with the proviso that the compound of formula I is not:
N-[1-(phenylmethyl)-1H-indol-5-yl]-Methanesulfonamide;
N-[1-[(4-fluorophenyl)methyl]-1H-indol-5-yl]-Methanesulfonamide;
N-[2,3-dihydro-1-(phenylmethyl)-1H-indol-5-yl]-Methanesulfonamide;
N-[1-(phenylmethyl)-1H-indol-5-yl]-N′-4-quinolinyl-Urea;
N-[1-(phenylmethyl)-1H-indol-5-yl]-N′-4-quinolinyl-Urea; or
1-(1-benzyl-5-indolinyl)-3-phenyl-Urea;
or salts thereof.

2. A compound according to claim 1, wherein at least one of R¹ or R^{1′} is a hydrogen atom.

3. A compound according to claim 2, wherein both R¹ and R^{1′} are hydrogen atoms.

4. A compound according to claim 1, wherein s is 0.

5. A compound according to claim 1, wherein s is 1.

6. A compound according to claim 1, wherein R² is a hydrogen atom.

7. A compound according claim 1, wherein R² is NO₂ or a halogen atom.

8. A compound according to claim 1, wherein U is NR¹¹.

9. A compound according to claim 8, wherein R¹¹ is a hydrogen atom.

10. A compound according to claim 1, wherein X is CO.

11. A compound according to claim 1, wherein X is SO₂.

12. A compound according to claim 1, wherein q is 0.

13. A compound according to claim 1, wherein q is 1.

14. A compound according to claim 13, wherein Z is an oxygen atom.

15. A compound according to claim 1, wherein R³ is selected from the group consisting of C₁₋₆-alk(en/yn)yl, C₃₋₈-cycloalk(en)yl, Ar, Ar—C₁₋₆-alk(en/yn)yl, Ar-oxy-C₁₋₆-alk(en/yn)yl, Ar—C₁₋₆-alk(en/yn)yloxy-C₁₋₆-alk(en/yn)yl and —NR¹²R^{12′}; with the proviso that when R³ is NR¹²R^{12′} then q is 0.

16. A compound according to claim 15, wherein R³ is NR¹²R^{12′}, q is 0 and R¹² and R^{12′} are independently selected from the group consisting of hydrogen, C₁₋₆-alk(en/yn)yl, Ar and Ar—C₁₋₆-alk(en/yn)yl, or R¹² and R^{12′} together with the nitrogen atom to which they are attached form a 4-8 membered saturated or unsaturated ring which optionally contains 1, 2 or 3 further heteroatoms.

17. A compound according to claim 1, wherein Y is of formula II, III, V, XXX, or XXXI.

18. A compound according to claim 17, wherein Y is of formula II or III and W is a sulphur atom.

19. A compound according to claim 17, wherein Y is of formula XXX and T is a nitrogen atom or an oxygen atom.

20. A compound according to claim 17, wherein Y is of formula XXXI and L is C or CH.

21. A compound according to claim 1, wherein each R⁵ is independently selected from the group consisting of C₁₋₆-alk(en/yn)yl, Ar, Ar-thio, Ar-oxy, halogen and halo-C₁₋₆-alk(en/yn)yl or two adjacent R⁵ together with the aromatic group to which they are attached form a 4-8 membered ring which optionally contains one or two heteroatoms.

22. A compound selected from the group consisting of:
N-[4-Chloro-1-(4-trifluoromethylbenzyl)-2,3-dihydro-1H-indol-5-yl]-3,3-dimethylbutyramide;
N-[4-Chloro-1-(5-chlorothiophen-2-ylmethyl)-2,3-dihydro-1H-indol-5-yl]-3,3-dimethylbutyramide;
[1-(4-Fluorobenzyl)-2,3-dihydro-1H-indol-5-yl]-carbamic acid propyl ester;
N-[1-(4-Fluorobenzyl)-2,3-dihydro-1H-indol-5-yl]-C-phenyl-methanesulfonamide;
4-Fluoro-N-[1-(4-fluorobenzyl)-2,3-dihydro-1H-indol-5-yl]-benzamide;
N-[1-(4-Fluorobenzyl)-2,3-dihydro-1H-indol-5-yl]-3,3-dimethylbutyramide;
N-[1-(4-Fluorobenzyl)-2,3-dihydro-1H-indol-5-yl]-2-thiophen-2-ylacetamide;
N-[1-(4-Fluorobenzyl)-2,3-dihydro-1H-indol-5-yl]-2-(4-fluorophenyl)-acetamide;
3-[1-(5-Chlorothiophen-2-ylmethyl)-2,3-dihydro-1H-indol-5-yl]-1,1-diisopropylurea;
Morpholine-4-carboxylic acid [1-(5-chlorothiophen-2-ylmethyl)-2,3-dihydro-1H-indol-5-yl]-amide;
Pyrrolidine-1-carboxylic acid [1-(5-chlorothiophen-2-ylmethyl)-2,3-dihydro-1H-indol-5-yl]-amide;
[1-(5-Chlorothiophen-2-ylmethyl)-2,3-dihydro-1H-indol-5-yl]-carbamic acid 2-benzyloxyethyl ester;
3-[1-(5-Chlorothiophen-2-ylmethyl)-2,3-dihydro-1H-indol-5-yl]-1-methyl-1-propylurea;
[1-(5-Chlorothiophen-2-ylmethyl)-2,3-dihydro-1H-indol-5-yl]-carbamic acid tert-butyl ester;
N-[1-(5-Chlorothiophen-2-ylmethyl)-2,3-dihydro-1H-indol-5-yl]-C-phenyl-methanesulfonamide;
Butane-1-sulfonic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-4-fluorobenzamide;
N-[1-(5-Chlorothiophen-2-ylmethyl)-2,3-dihydro-1H-indol-5-yl]-2,2-dimethylpropionamide;
N-[1-(5-Chlorothiophen-2-ylmethyl)-2,3-dihydro-1H-indol-5-yl]-2-phenoxyacetamide;
N-[1-(5-Chlorothiophen-2-ylmethyl)-2,3-dihydro-1H-indol-5-yl]-3,3-dimethylbutyramide;
N-[1-(5-Chlorothiophen-2-ylmethyl)-2,3-dihydro-1H-indol-5-yl]-butyramide;
Cyclopentanecarboxylic acid [1-(5-chlorothiophen-2-ylmethyl)-2,3-dihydro-1H-indol-5-yl]-amide;
N-[1-(5-Chlorothiophen-2-ylmethyl)-2,3-dihydro-1H-indol-5-yl]-2-thiophen-2-ylacetamide;
N-[1-(5-Chlorothiophen-2-ylmethyl)-2,3-dihydro-1H-indol-5-yl]-isonicotinamide;
N-[1-(5-Chlorothiophen-2-ylmethyl)-2,3-dihydro-1H-indol-5-yl]-4-dimethylaminobenzamide;
N-[1-(5-Chlorothiophen-2-ylmethyl)-2,3-dihydro-1H-indol-5-yl]-2-(4-fluorophenyl)-acetamide;
N-[1-(5-Chlorothiophen-2-ylmethyl)-2,3-dihydro-1H-indol-5-yl]-6-trifluoromethylnicotinamide;
1-tert-Butyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-ethylurea;
1-Benzyl-3-[1-(5-chlorothiophen-2-ylmethyl)-2,3-dihydro-1H-indol-5-yl]-urea;
1-[1-(5-Chlorothiophen-2-ylmethyl)-2,3-dihydro-1H-indol-5-yl]-3-phenethylurea;
1-[1-(5-Chlorothiophen-2-ylmethyl)-2,3-dihydro-1H-indol-5-yl]-3-thiophen-2-ylurea;
1-[1-(5-Chlorothiophen-2-ylmethyl)-2,3-dihydro-1H-indol-5-yl]-3-thiophen-3-ylurea;
[1-(5-Chlorothiophen-2-ylmethyl)-2,3-dihydro-1H-indol-5-yl]-carbamic acid propyl ester;
2,2-Dimethyl-N-[6-nitro-1-(4-trifluoromethylbenzyl)-2,3-dihydro-1H-indol-5-yl]-propionamide;
N-[1-(5-Chlorothiophen-2-ylmethyl)-6-nitro-2,3-dihydro-1H-indol-5-yl]-2,2-dimethylpropionamide;
2-(4-Fluorophenyl)-N-[6-nitro-1-(4-trifluoromethylbenzyl)-2,3-dihydro-1H-indol-5-yl]-acetamide;
N-[1-(5-Chlorothiophen-2-ylmethyl)-6-nitro-2,3-dihydro-1H-indol-5-yl]-2-(4-fluorophenyl)-acetamide;
N-[1-(5-Chlorothiophen-2-ylmethyl)-6-nitro-2,3-dihydro-1H-indol-5-yl]-3,3-dimethylbutyramide;
N-[6-Amino-1-(5-chlorothiophen-2-ylmethyl)-2,3-dihydro-1H-indol-5-yl]-3,3-dimethylbutyramide;
N-[6-Amino-1-(4-trifluoromethylbenzyl)-2,3-dihydro-1H-indol-5-yl]-2,2-dimethylpropionamide;
N-[6-Amino-1-(5-chlorothiophen-2-ylmethyl)-2,3-dihydro-1H-indol-5-yl]-2,2-dimethylpropionamide;
N-[6-Amino-1-(4-trifluoromethylbenzyl)-2,3-dihydro-1H-indol-5-yl]-2-(4-fluorophenyl)-acetamide;
N-[6-Amino-1-(4-trifluoromethylbenzyl)-2,3-dihydro-1H-indol-5-yl]-3,3-dimethylbutyramide;
N-[6-Amino-1-(4-fluorobenzyl)-2,3-dihydro-1H-indol-5-yl]-3,3-dimethylbutyramide;
N-[6-Amino-1-(3-fluoro-4-trifluoromethylbenzyl)-2,3-dihydro-1H-indol-5-yl]-3,3-dimethylbutyramide;
N-[1-(5-Chlorothiophen-2-ylmethyl)-1H-indol-5-yl]-3,3-dimethylbutyramide;
N-[6-Bromo-1-(4-trifluoromethylbenzyl)-2,3-dihydro-1H-indol-5-yl]-3,3-dimethylbutyramide;
N-[6-Bromo-1-(5-chlorothiophen-2-ylmethyl)-2,3-dihydro-1H-indol-5-yl]-3,3-dimethylbutyramide;
N-[1-(4-Chlorobenzyl)-2,3-dihydro-1H-indol-5-yl]-3,3-dimethylbutyramide;
3,3-Dimethyl-N-[1-(4-trifluoromethylbenzyl)-2,3-dihydro-1H-indol-5-yl]-butyramide;
N-[1-(4-Isopropylbenzyl)-2,3-dihydro-1H-indol-5-yl]-3,3-dimethylbutyramide;
N-[1-(3-Fluoro-4-trifluoromethylbenzyl)-2,3-dihydro-1H-indol-5-yl]-3,3-dimethylbutyramide;
N-[1-(6-Chlorobenzo[1,3]dioxol-5-ylmethyl)-2,3-dihydro-1H-indol-5-yl]-3,3-dimethylbutyramide;
N-[1-(3,5-Dimethyl-1-phenyl-1H-pyrazol-4-ylmethyl)-2,3-dihydro-1H-indol-5-yl]-3,3-dimethylbutyramide;
N-[1-(2-Chloro-5-trifluoromethylbenzyl)-2,3-dihydro-1H-indol-5-yl]-3,3-dimethylbutyramide;
N-{1-[5-(4-Chlorophenoxy)-1,3-dimethyl-1H-pyrazol-4-ylmethyl]-2,3-dihydro-1H-indol-5-yl}-3,3-dimethylbutyramide;
3,3-Dimethyl-N-[1-(6-p-tolyloxy-pyridin-3-ylmethyl)-2,3-dihydro-1H-indol-5-yl]-butyramide;
N-{1-[6-(4-Chlorophenylsulfanyl)-pyridin-3-ylmethyl]-2,3-dihydro-1H-indol-5-yl}-3,3-dimethylbutyramide;
N-{1-[6-(4-Cyanophenoxy)-pyridin-3-ylmethyl]-2,3-dihydro-1H-indol-5-yl}-3,3-dimethylbutyramide;
3,3-Dimethyl-N-[1-(6-trifluoromethylpyridin-3-ylmethyl)-2,3-dihydro-1H-indol-5-yl]-butyramide;
3,3-Dimethyl-N-[1-(3-methyl-benzo[b]thiophen-2-ylmethyl)-2,3-dihydro-1H-indol-5-yl]-butyramide;
N-[1-(6-Fluoro-4H-benzo[1,3]dioxin-8-ylmethyl)-2,3-dihydro-1H-indol-5-yl]-3,3-dimethylbutyramide;
3,3-Dimethyl-N-[1-(6-phenoxypyridin-3-ylmethyl)-2,3-dihydro-1H-indol-5-yl]-butyramide;
3,3-Dimethyl-N-[1-(3-methyl-5-phenyl-isoxazol-4-ylmethyl)-2,3-dihydro-1H-indol-5-yl]-butyramide;
N-(1-Benzo[b]thiophen-2-ylmethyl-2,3-dihydro-1H-indol-5-yl)-3,3-dimethylbutyramide;
N-{1-[1-(4-Fluorophenyl)-5-methyl-1H-pyrazol-4-ylmethyl]-2,3-dihydro-1H-indol-5-yl}-3,3-dimethylbutyramide;
3,3-Dimethyl-N-[1-(5-methylthiophen-2-ylmethyl)-2,3-dihydro-1H-indol-5-yl]-butyramide;
3,3-Dimethyl-N-[1-(4-pyrrol-1-yl-benzyl)-2,3-dihydro-1H-indol-5-yl]-butyramide;
N-[1-(4-Chlorobenzyl)-2,3-dihydro-1H-indol-5-yl]-2-(4-fluorophenyl)-acetamide;
2-(4-Fluorophenyl)-N-[1-(4-trifluoromethylbenzyl)-2,3-dihydro-1H-indol-5-yl]-acetamide;
2-(4-Fluorophenyl)-N-[1-(4-isopropylbenzyl)-2,3-dihydro-1H-indol-5-yl]-acetamide;
2-(4-Fluorophenyl)-N-[1-(3-fluoro-4-trifluoromethylbenzyl)-2,3-dihydro-1H-indol-5-yl]-acetamide;
N-[1-(6-Chlorobenzo[1,3]dioxol-5-ylmethyl)-2,3-dihydro-1H-indol-5-yl]-2-(4-fluorophenyl)-acetamide;
N-[1-(3,5-Dimethyl-1-phenyl-1H-pyrazol-4-ylmethyl)-2,3-dihydro-1H-indol-5-yl]-2-(4-fluorophenyl)-acetamide;
N-[1-(2-Chloro-5-trifluoromethylbenzyl)-2,3-dihydro-1H-indol-5-yl]-2-(4-fluorophenyl)-acetamide;
N-{1-[5-(4-Chlorophenoxy)-1,3-dimethyl-1H-pyrazol-4-ylmethyl]-2,3-dihydro-1H-indol-5-yl}-2-(4-fluorophenyl)-acetamide;
N-{1-[6-(4-Cyanophenoxy)-pyridin-3-ylmethyl]-2,3-dihydro-1H-indol-5-yl}-2-(4-fluorophenyl)-acetamide;
2-(4-Fluorophenyl)-N-[1-(3-methyl-benzo[b]thiophen-2-ylmethyl)-2,3-dihydro-1H-indol-5-yl]-acetamide;
N-[1-(6-Fluoro-4H-benzo[1,3]dioxin-8-ylmethyl)-2,3-dihydro-1H-indol-5-yl]-2-(4-fluorophenyl)-acetamide;
2-(4-Fluorophenyl)-N-[1-(6-phenoxypyridin-3-ylmethyl)-2,3-dihydro-1H-indol-5-yl]-acetamide;
N-(1-Benzo[b]thiophen-2-ylmethyl-2,3-dihydro-1H-indol-5-yl)-2-(4-fluorophenyl)-acetamide;
2-(4-Fluorophenyl)-N-{1-[1-(4-fluorophenyl)-5-methyl-1H-pyrazol-4-ylmethyl]-2,3-dihydro-1H-indol-5-yl}-acetamide;
2-(4-Fluorophenyl)-N-[1-(5-methylthiophen-2-ylmethyl)-2,3-dihydro-1H-indol-5-yl]-acetamide; and
2-(4-Fluorophenyl)-N-[1-(4-pyrrol-1-yl-benzyl)-2,3-dihydro-1H-indol-5-yl]-acetamide, or
a pharmaceutically acceptable salt thereof.

23. A pharmaceutical composition comprising a compound according to claim 1 and one or more pharmaceutically acceptable carriers or diluents.

24. A method of increasing ion flow in a potassium channel of a mammal, comprising administering to said mammal a compound of formula I 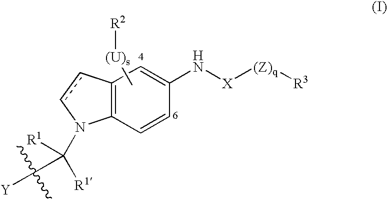
wherein
the dotted line represents an optional bond;
R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or
R¹ and R^{1′} together with the carbon atom to which they are attached form a 3-8 membered saturated or unsaturated ring which optionally contains 1 or 2 heteroatoms;
s is 0 or 1;
U is O, NR¹¹, S, SO₂, SO₂NR¹¹, CO—O or CO—NR¹¹; wherein R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or R² and R¹¹ together with the nitrogen atom to which they are attached form a 4-8 membered saturated or unsaturated ring which optionally contains 1, 2 or 3 further heteroatoms;
R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′}—C₁₋₆-alk(en/yn)yl, NR¹⁰R^{10′}—C₃₋₈-cycloalk(en)yl and NR¹⁰R^{10′}—C₃₋₈-cycloalk(en)yl-C₁₋₆-alk(en/yn)yl; wherein
R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹⁰ and R^{10′} together with the nitrogen atom to which they are attached form a 4-8 membered saturated or unsaturated ring which optionally contains 1, 2 or 3 further heteroatoms;
with the proviso that when R² is NO₂, halogen or cyano then s is 0; and
with the proviso that when R² is a hydrogen atom or acyl and s is 1 then U is NR¹¹, O or S;
wherein the group —(U)ₛ—R² is linked to position 4 or 6 of the indole or indoline;
q is 0 or 1;
Z is O or S;
X is CO or SO₂; with the proviso that q is 0 when X is SO₂;
R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar—C₁₋₆-alk(en/yn)yl, Ar—C₃₋₈-cycloalk(en)yl, Ar-heterocycloalk(en)yl, Ar—C₃₋₈-cycloalk(en)yl-C₁₋₆-alk(en/yn)yl, Ar—C₁₋₆-alk(en/yn)yl-C₃₋₈-cycloalk(en)yl, Ar—C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar—C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl and —NR¹²R^{12′}, optionally substituted NR¹²R^{12′}—C₁₋₆-alk(en/yn)yl, optionally substituted NR¹²R^{12′}—C₃₋₈-cycloalk(en)yl, optionally substituted NR¹²R^{12′}—C₃₋₈-cycloalk(en)yl-C₁₋₆-alk(en/yn)yl; wherein
R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar—C₃₋₈-cycloalk(en)yl, Ar—C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or
R¹² and R^{12′} together with the nitrogen atom to which they are attached form a 4-8 membered saturated or unsaturated ring which optionally contains 1, 2 or 3 further heteroatoms;
with the proviso that when R³ is NR¹²R^{12′} then q is 0;
and
Y represents a group of formula II, III, IV, V, VI, XXX or XXXI: 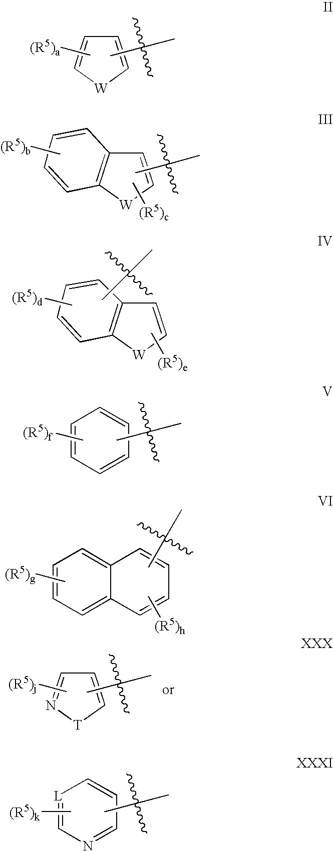
W is O or S;
T is N, NH or O;
L is N, C or CH;
a is 0, 1, 2 or 3;
b is 0, 1, 2, 3 or 4;
c is 0 or 1;
d is 0, 1, 2 or 3;
e is 0, 1 or 2;
f is 0, 1, 2, 3, 4 or 5;
g is 0, 1, 2, 3 or 4;
h is 0, 1, 2 or 3;
j is 0, 1, 2 or 3; with the proviso that when T is a nitrogen atom then j is 0, 1, 2 or 3; and when T is NH or an oxygen atom then j is 0, 1 or 2;
k is 0, 1, 2, 3 or 4; and
each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar—C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸, or
two adjacent R⁵ together with the aromatic group to which they are attached form a 4-8 membered ring which optionally contains one or two heteroatoms;
R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar;
R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl;
and
R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′} then R⁵ is not —S—R⁸;
or salts thereof.

25. The method of claim 24 wherein administration of said compound is for the prevention, treatment or inhibition of a disorder or condition being responsive to an increased ion flow in a potassium channel.

26. The method of claim 25, wherein said disorder or disease is a seizure disorder.

27. The method of claim 25 wherein the disorder or condition is selected from the group consisting of neuropathic and migraine pain disorders.

28. The method of claim 25 wherein the disorder or condition is an anxiety disorder.

29. The method of claim 25 wherein the disorder or condition is a neurodegenerative disorder.

30. The method of claim 25 wherein the disorder or condition is a neuronal hyperexcitation state.

31. The method of claim 24 wherein the mammal is a human.

32. The method of claim 25 wherein the disorder or condition is a disorder or condition of the central nervous system.

33. The method of claim 26 wherein the seizure disorder is selected from the group consisting of convulsions, epilepsy and status epileptus.

34. The method of claim 27 wherein the neuropathic or migraine pain disorder is selected from the group consisting of allodynia, hyperalgesic pain, phantom pain, neuropathic pain related to diabetic neuropathy and neuropathic pain related to migraine.

35. The method of claim 28 wherein the anxiety disorder is selected from the group consisting of anxiety, generalized anxiety disorder, panic anxiety, obsessive compulsive disorder, social phobia, performance anxiety, post-traumatic stress disorder, acute stress reaction, adjustment disorders, hypochondriacal disorders, separation anxiety disorder, agoraphobia, specific phobias, anxiety disorder due to general medical condition and substance-induced anxiety disorder.

36. The method of claim 29 wherein the neurodegenerative disorder is selected from the group consisting of Alzheimer's disease, Huntington's chorea, multiple sclerosis, amyotrophic lateral sclerosis, AIDS-induced encephalopathy and other infection-related encephalopathies being caused by rubella viruses, herpes viruses, borrelia and by unknown pathogens, Creutzfeld-Jakob disease, Parkinson's disease and trauma-induced neurodegenerations.

37. The method of claim 30 wherein the neuronal hyperexcitation state is due to medicament withdrawal or intoxication.
